SEQUENCE LISTING (107 sequences)

SEQ ID NO: 1 - DNA, 3434 bp, Homo sapiens
  CDS (719)..(2338)
gaggtacacgaggagttactggaaacggcgcttctgtggaggagccgggggggatggggagtagagggagggggccctgttgcctcagcgccccgaggtcgtggagcggcagcagctgcagccggagcagcaccagcaacagcaacagcgagcgggacggagttaggaccgctcggagcgcacaggtctcgagggtgttggtgccagaagaaaagaatgattgatgggaaacagacaccgggctatagacactcatccttttgcttcagatactgatatctcagcctgcttgagcatcccttgtgagctgtgaacattgaggatcactcagggttatcggatgtacaacgggagagccatcgctttgctaaattattatctgcaattggacatcttttacaaaaaccaaactagacctgagtctaatagatatgttctaagacaaagaaaaagctgcaagttgttaacgcctaacacacaagtatgttaggcttccaccaaagtcctcaatatacctgaatacgcacaatatcttaactcttcatatttggttttgggatctgctttgaggtcccatcttcatttaaaaaaaaatacagagacctacctacccgtacgcatacatacatatgtgtatatatatgtaaactagacaaagatcgcagatcataaagcaagctctgctttagtttccaagaagattacaaagaatttagagatgtatttgtcaagattcctgtcgattcatgccctttgggttacggtgtcctcagtgatgcagccctaccctttggtttggggacattatgatttgtgtaagactcagatttacacggaagaagggaaagtttgggattacatggcctgccagccggaatccacggacatgacaaaatatctgaaagtgaaactcgatcctccggatattacctgtggagaccctcctgagacgttctgtgcaatgggcaatccctacatgtgcaataatgagtgtgatgcgagtacccctgagctggcacacccccctgagctgatgtttgattttgaaggaagacatccctccacattttggcagtctgccacttggaaggagtatcccaagcctctccaggttaacatcactctgtcttggagcaaaaccattgagctaacagacaacatagttattacctttgaatctgggcgtccagaccaaatgatcctggagaagtctctcgattatggacgaacatggcagccctatcagtattatgccacagactgcttagatgcttttcacatggatcctaaatccgtgaaggatttatcacagcatacggtcttagaaatcatttgcacagaagagtactcaacagggtatacaacaaatagcaaaataatccactttgaaatcaaagacaggttcgcgttttttgctggacctcgcctacgcaatatggcttccctctacggacagctggatacaaccaagaaactcagagatttctttacagtcacagacctgaggataaggctgttaagaccagccgttggggaaatatttgtagatgagctacacttggcacgctacttttacgcgatctcagacataaaggtgcgaggaaggtgcaagtgtaatctccatgccactgtatgtgtgtatgacaacagcaaattgacatgcgaatgtgagcacaacactacaggtccagactgtgggaaatgcaagaagaattatcagggccgaccttggagtccaggctcctatctccccatccccaaaggcactgcaaatacctgtatccccagtatttccagtattggtaattgtgaatgcttcggccactccaatcgatgcagttatatcgatctgctaaatacagtcatttgcgtgagctgtaaacacaacactagagggcagcactgtgagttatgcaggctgggctacttcagaaatgcttctgcacaactggacgatgagaatgtgtgcatagagtgttattgtaaccctttgggctcaatccatgatcgttgtaatggctcaggattttgtgagtgtaagactggaacaacagggcctaagtgtgatgagtgtctgccgggaaattcctggcactacggctgtcaaccgaatgtctgcgacaacgagctcctgcactgccagaacggagggacgtgccacaacaacgtgcgctgcctgtgcccggccgcatacacgggcatcctctgcgagaagctgcggtgcgaggaggctggcagctgcggctccgactctggccagggcgcgcccccgcacggctccccagcgctgctgctgctgaccacgctgctgggaaccgccagccccctggtgttctaggtgtcacctccagccacaccggacgggcctgtgccgtggggaagcagacacaacccaaacatttgctactaacataggaaacacacacatacagacacccccactcagacagtgtacaaactaagaaggcctaactgaactaagccatatttatcacccgtggacagcacatccgagtcaagactgttaatttctgactccagaggagttggcagctgttgatattatcactgcaaatcacattgccagctgcagagcatattgtggattggaaaggctgcgacagccccccaaacaggaaagacaaaaaacaaacaaatcaaccgacctaaaaacattggctactctagcgtggtgcgccctagtacgactccgcccagtgtgtggaccaaccaaatagcattctttgctgtcaggtgcattgtgggcataaggaaatctgttacaagctgccatattggcctgcttccgtccctgaatcccttccaacctgtgctttagtgaacgttgctctgtaacccttgttggttgaaagatttctttgtctgatgttagtgatgcacatgtgtaacagccccctctaaaagcgcaagccagtcatacccctgtatatcttagcagcactgagtccagtgcgagcacacacccactatacaagagtggctataggaaaaaagaaagtgtatctatccttttgtattcaaatgaagttatttttcttgaactactgtaatatgtagattttttgtattattgccaatttgtgttaccagacaatctgttaatgtatctaattcgaatcagcaaagactgacattttattttgtcctctttcgttctgttttgtttcactgtgcagagatttctctgtaagggcaacgaacgtgctggcatcaaagaatatcagtttacatatataacaagtgtaataagattccaccaaaggacattctaaatgttttcttgttgctttaacactggaagatttaaagaataaaaactcctgcataaacaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 539 aa, Homo sapiens
MYLSRFLSIHALWVTVSSVMQPYPLVWGHYDLCKTQIYTEEGKVWDYMACQPESTDMTKYLKVKLDPPDITCGDPPETFCAMGNPYMCNNECDASTPELAHPPELMFDFEGRHPSTFWQSATWKEYPKPLQVNITLSWSKTIELTDNIVITFESGRPDQMILEKSLDYGRTWQPYQYYATDCLDAFHMDPKSVKDLSQHTVLEIICTEEYSTGYTTNSKIIHFEIKDRFAFFAGPRLRNMASLYGQLDTTKKLRDFFTVTDLRIRLLRPAVGEIFVDELHLARYFYAISDIKVRGRCKCNLHATVCVYDNSKLTCECEHNTTGPDCGKCKKNYQGRPWSPGSYLPIPKGTANTCIPSISSIGNCECFGHSNRCSYIDLLNTVICVSCKHNTRGQHCELCRLGYFRNASAQLDDENVCIECYCNPLGSIHDRCNGSGFCECKTGTTGPKCDECLPGNSWHYGCQPNVCDNELLHCQNGGTCHNNVRCLCPAAYTGILCEKLRCEEAGSCGSDSGQGAPPHGSPALLLLTTLLGTASPLVF

SEQ ID NO: 3 - DNA, 3219 bp, Homo sapiens
  CDS (119)..(2458)
agactctcaggttgatgcagtgttccctcccacaactctgacatgtatataaattctgagctctccaaagcccactgccagttctcttcggggactaactgcaacggagagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctataagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaacaggactttatacctcttttactgtaacaagtactcattaaaggaaattgaatgaaattaaaaaaaaaaa

SEQ ID NO: 4 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 5 - DNA, 7616 bp, Homo sapiens
  CDS (363)..(6968)
attacagaggaaggagctcgctatataagccagccaaagttggctgcaccggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttaaaaaaaaaaaaaaaaaa

SEQ ID NO: 6 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 7 - DNA, 6314 bp, Homo sapiens
  CDS (151)..(2193)
gtcggcgaggagggtccggccggagttgaaggattgaactttccggctcagtcgcggcgtctgcctggtcctcagcagtgcagccccggcgcggagcagggagcctcggcccgcgcccggcgccctcgccctcgccctcgacccgcagccatggtgcccggggtgcccggcgcggtcctgaccctctgcctctggctggcggcctccagcggctgcctggcggccggccccggcgcggctgctgcgcggcggctggacgagtcgctgtctgccgggagcgtccagcgcgctcgctgcgcctccaggtgcctgagcctgcagatcactcgcatctccgccttcttccagcacttccagaacaatggttccctggtttggtgccagaatcacaagcaatgttctaagtgcctggagccctgcaaggaatcaggggacctgaggaaacaccagtgccaaagcttttgtgagcctctcttccccaagaagagctacgaatgcttgaccagctgtgagttcctcaaatacatcctgttggtgaagcagggggactgtccggctcctgagaaagccagtggatttgcggccgcctgtgttgaaagctgcgaagttgacaatgagtgctctggggtgaagaaatgttgttcgaatgggtgtggacacacctgtcaagtacccaagactctgtacaaaggtgtccccctgaagcccagaaaagagttacgatttacagaactgcagtctggacagctggaggttaagtggtcctcgaaattcaatatttctattgagcctgtgatctatgtggtacaaagaagatggaattatggaatccatcctagcgaagatgacgccactcactggcagacagtggcccagaccacagacgagcgagttcaactgactgacataagacccagccgatggtaccagtttcgagtggctgctgtgaatgtgcatggaactcgaggcttcactgcccccagcaaacacttccgttcttccaaagatccatctgccccaccagcaccggctaacctccggctggccaactccaccgtcaacagtgatgggagtgtgaccgtcactatagtttgggatctccccgaggagccggacatccctgtgcatcattacaaggtcttttggagctggatggtcagcagtaagtctcttgtcccaacaaagaagaagcggagaaagactacggatgggtttcaaaattctgtgatcctggagaaactccagccagactgtgactatgttgtggaattgcaagccataacgtactggggacagacacggctgaagagtgcaaaggtgtcccttcacttcacatcgacacatgcaaccaacaacaaagaacagcttgtgaaaactagaaaaggtggaattcaaacacaactcccttttcaaagacgacgacccactcgcccgctggaagtcggagctcccttctatcaggatggccaactgcaagttaaagtctactggaagaagacagaagatcccactgtcaaccgatatcatgtgcggtggtttcctgaagcgtgtgcccacaacagaacaaccggatcagaggcatcatctggcatgacccacgaaaattacataattcttcaagatctgtcattttcctgcaagtataaggtgactgtccaaccaatacggccaaaaagtcactccaaggcagaagctgttttcttcactactccaccatgctctgctcttaaggggaagagccacaagcctgttggctgcctgggcgaagcaggtcatgttctttctaaggtgctagctaagcctgagaacctttctgcttcattcatcgtccaggatgtgaacatcaccggtcacttttcttggaagatggccaaggccaatctctatcagcccatgactgggtttcaagtgacttgggctgaggtcactacggaaagcagacagaacagcctacccaacagcattatttcacagtcccagatcctgccttccgatcattatgtcctaacagtgcccaatctgagaccatctactctttaccgactggaagtgcaagtgctgaccccaggaggggaggggccggccaccatcaagacgttccggacgccggagctcccaccctcttcagcacacagatctcatcttaagcatcgtcatccacatcattacaagccttctccagaaagatactaaactgttcaaaaagattttgtgaaattgcacagatgtgtaagcttgttgaacttcggccacgagacatgcacacttccagaggcagtgggaactgctcagaggcccggactctcctatgtgactttagtgcaggaagaacttctgtcaatcatggacgcatctggagacaagtgagaaacagtagattggtgaagacagacaccagttccctacaagcatggagaaaatgaagaataggcctgtttaatgctaaattttgttttcatgtatggtgtcgctcatttctattgaattacaacagaactcagttttccctgaatttggagcaccaaactccgccccaaaaaggagagtaacaaatacacaattcacacataacactaagcgtaaatctaatcaataaaatatatttttgactaaattattgattcgatatgaaaaatcaactaagattacacagctttgtttttttgaatctttcctaagatcatttttatcctaggtgatttttaaatgaaaatgtgtaatctaaaatataccagcgaatttaaatctaaaaatgctcctactttaagtaccttgtgctgctctttatgcaaaggtaaatcaaagttccctctataaattatgatttacaaaagacacccaagccagaggaactcaatgaaataagctgctaatcagattttaccttggagaaatgaaaattatttcttggggatgccttttaatatttgatcctattatgtgagagattttcctgatatgttatcttatttatattttcccttattttcctcaatgcagataatagcttttggtgcacttttgtttcaccatctgaaaattcacaaaacttcttgcttcaaatgaaaaaatcccaactattgagcatgtttaaatctttgcagagatttgccttttcttaatcaaagaaaggtctttgtgtgctagaatattattggtaatgttttaaaaattcctttgattgatagagaaggacagttatttgcatttaattcacccatatgctttcaaatctagtatatcttactttttggaaatgttttatgctacaaattagtgccttgtagcatgaacttaagtcaaaacgtgttatcaatatagagtgttgcagtgtatattgtaacaacctaaaacgcagagaagtttaatttaatactgttttttttcttgaaggaatactcacatacatggtttgaaatgtgcatagatatgcatgtctatataattataaatgcatgtgtatatatatgcaaatatatgtacatatacatgtatatacacacagacacatgcatatacatgaatataccttgagcatgaatccctggagaaatcgttttcgtagctcaccaatggtgagtaaagatacagctcttttaaaggtcataaggataatatattttccccatcaatgctgattctgagaaaagagcaatttatcaaaattaaacactgtaaaagaaaggtgtccatatgtctttacctacctaagtaaaacaggaagaaaatcagtaacattatccttaggttttgacaatggtacttgcttcttgttgttttattgtttcctgaattcatgcagatgcctggccattcctgggaagagtggataactcagaagtcactgtactccacagagcctcactgcagtgtctaaaggtagatgcaaattaaaatgcagggaaaataacttttctgatgttgatgcatgtctttgggaaacacatttataaacatggatacctgataatagatattgaaacccatttcctgtgtgttaaaatatttaaaaagtggatattccaggaatgttttgcagctttgtacaagtaacataaattggacacctcagaatgaaagttcatgttggttctgaatggttcactgcagctcctgtcacaagctgggatggatttatcacattgagttatgaaattacctggttctaagaatttttgagtggcaaaaatagaaaacaatcttcatttgaaaacatccctaagcttgaataaatggataccatagatagcttctcttttttattctggtgtcattaccagcatctgaatttcaagttcttaaaatttcaaaaattaaaatttttcattattagctatccatttatcttttacatgaacttgtcatgaacaaattcaaatgtttatgccagcaaatttttgtactgttgcatagttaaaaatgctgggagtctctgcatagatacaaaatattattaaattattacataaatttaattttataaaatttaatcatgcttcttttgtctgggtaatagacattggacagatatttttagttcagatggtgattctgaagcttacatctcccttaaaaaaatctaaagcagctcttatgggcttctaattttaatataaataaataatttaaattttattggtgttattggaagaaaaatgctattaatgggctaataaaaaacatgtgtttctcttatggattttaataagctccagtattattcaaatgatcaaaaatatagttataattttttgaattttaaaaatgtgattgctctaataaagaataaaatctatgctttttaacaaacatagttttggtgcctaattctgtaatatgttttattgaaattagattcatttctctaatgtgagaaaaatatatccagtaatagtattgactgtttaaaaaattgagctcatcaaaaatattgtcatcaaatacaggtggttaatctgacatacattgcagttacatgcattatttttatttacaacatttgctccttaatgatgaatttatctgtgttaccctgtttttctacctggaactccatagaatgatgtttgcaaaccaacatgtgctcttttcagtcattcactgttttaatatgacatggtagagaagataaggtttatggcaggtaattttttgtaatgtgtattaaacgaagttcaaagattagaaatacatctgtgtcctgaaaaccttagatacatagccgactgtatacagaggttcatctcaacctcaacactattgacttttggggctggatagttctctgttgtgggggtttgtcttgtgcactgtaggtttttagtagcatccacactttctcctcaccagatgccagttgcaccctcccccaagttgagacaaccaaaaatgtctccagatattgccagctaccccttgagggatggtacctctggttgagaaccattgctagagaatgatctttactgaatttgccctttataagaaacccagtgaatttctagagcaagtcccaaaaactaagggacagctaagaagttattatggttgacttcaaaggcctaaactgtgttttttatgtccactaaacaacttgattaaaagacggaattttgactcgtgtctgtatcatacaagtacaaatactaattttgccctatgtatccgtaaatgtcatttgtgattttgacttatttatttaatgccctttcttatgccgtgggttttcaagtttactcatttctatggttgcaaataactctaaaacttattatataaactttcatattataggcagaacacaatggctaaatatctgttgcatgtactttaaagtttattataaaatataaacagatatataaagatgttgactcttacctgtgattttgcatggtcagactcggtgtcaggtacggagaggattctcatgactgtcttacctctactgaatattctagtgagttatatgatttacggagtgattaacagaggtctatataaagttacttttcccctttacttaattatattgtagtgtgcagataacaaaactgctaccttctcatccaagtggtctgtagaattcatgtcccttacagtggtcatttaaagtcaatatttatttatgtatgtaataaaaaaagttggatttttgtgtatgtctgtcacattatttagagagaagtaatcttgtaaaaatgttttgtaaaaaacaaaaaagtattgtaaatagtcttgatattctgtgactcattattttcatgttagagtttgtacatactggttcaataataaagtatccttaaaccaga

SEQ ID NO: 8 - protein, 680 aa, Homo sapiens
MVPGVPGAVLTLCLWLAASSGCLAAGPGAAAARRLDESLSAGSVQRARCASRCLSLQITRISAFFQHFQNNGSLVWCQNHKQCSKCLEPCKESGDLRKHQCQSFCEPLFPKKSYECLTSCEFLKYILLVKQGDCPAPEKASGFAAACVESCEVDNECSGVKKCCSNGCGHTCQVPKTLYKGVPLKPRKELRFTELQSGQLEVKWSSKFNISIEPVIYVVQRRWNYGIHPSEDDATHWQTVAQTTDERVQLTDIRPSRWYQFRVAAVNVHGTRGFTAPSKHFRSSKDPSAPPAPANLRLANSTVNSDGSVTVTIVWDLPEEPDIPVHHYKVFWSWMVSSKSLVPTKKKRRKTTDGFQNSVILEKLQPDCDYVVELQAITYWGQTRLKSAKVSLHFTSTHATNNKEQLVKTRKGGIQTQLPFQRRRPTRPLEVGAPFYQDGQLQVKVYWKKTEDPTVNRYHVRWFPEACAHNRTTGSEASSGMTHENYIILQDLSFSCKYKVTVQPIRPKSHSKAEAVFFTTPPCSALKGKSHKPVGCLGEAGHVLSKVLAKPENLSASFIVQDVNITGHFSWKMAKANLYQPMTGFQVTWAEVTTESRQNSLPNSIISQSQILPSDHYVLTVPNLRPSTLYRLEVQVLTPGGEGPATIKTFRTPELPPSSAHRSHLKHRHPHHYKPSPERY

SEQ ID NO: 9 - DNA, 1497 bp, Homo sapiens
  CDS (196)..(1152)
aaagtgctgggattacaggcatgagccgccgcgccccgccccacgctcagtcttgaaattgtctggaacgggaaacggcaaacagcgagatatccgagcgagagtcccgccctgcatcagtttgcggaaccgccttggtagaaggagagaaggggagtggaggaagcacgggactggagggaccaaagttccccgatggcggcccaggggtgcgcggcatcgcggctgctccagctgctgctgcagcttctgcttctactgttgctgctggcggcgggcggggcgcgcgcgcggtggcgcggggagggcaccagcgcacacttgcgggacatcttcctgggccgctgcgccgagtaccgcgcactgctgagtcccgagcagcggaacaagaactgcacagccatctgggaagcctttaaagtggcgctggacaaggatccctgctccgtgctgccctcagactatgacctttttattaacttgtccaggcactctattcccagagataagtccctgttctgggaaaatagccacctccttgttaacagctttgcagacaacacccgtcgttttatgcccctgagcgatgttctgtatggcagggttgcagatttcttgagctggtgtcgacagaaaaatgactctggactcgattaccaatcctgccctacatcagaagactgtgaaaataatcctgtggattccttttggaaaagggcatccatccagtattccaaggatagttctggggtgatccacgtcatgctgaatggttcagagccaacaggagcctatcccatcaaaggtttttttgcagattatgaaattccaaacctccagaaggaaaaaattacacgaatcgagatctgggttatgcatgaaattgggggacccaatgtggaatcctgcggggaaggcagcatgaaagtcctggaaaagaggctgaaggacatggggttccagtacagctgtattaatgattaccgaccagtgaagctcttacagtgcgtggaccacagcacccatcctgactgtgccttaaagtcggcagcagccgctactcaaagaaaagccccaagtctttatacagaacaaagggcgggtcttatcattcccctctttctggtgctggcttccaggactcaactgtaactggaaactgtgttgctctaaccctcctccagccctgcagcctccccttgcagtcatcattcgtgttctgtgtataccaaatgattctgttatctaaagaagctttttgctgggaaaacgatgtcctgaaaatggtatttcaatgaggcatatgttcaggatttcagaaacaagaagttagttctatttagcaggttaaaaaatgctgcattagaattaaagcaagttattttcttatttgtataatgacacaaagcattgggagtcagactgcttgtatattatcaaacattttaagagaattctaataaagctgtattttacatcaaaaaaaaaaaaaaaaaa

SEQ ID NO: 10 - protein, 318 aa, Homo sapiens
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL

SEQ ID NO: 11 - DNA, 3020 bp, Homo sapiens
  CDS (212)..(1045)
cacgcccctgggcggtgcgcgcgggcggtgggtaggccgggcaggctcacgtgatgcgggccccgcggcccgccatataaacccgcgcgcccgccaggcgctgcggccgtcccgggccgtgactcctcctttcccccgccccgcctccgttcggagagccggcgggcgggcgcctctcggccaggaagcgcctcttggacgcgtgtgaccgatgcccagattgcacgaccacttctggagctgctcctgtgcgcacagcgcgaggcgccgaggccccccgcgagccagcgccgcggggctggcggccaaggttggggagatgatcaacgtttccgtgtccgggccctccctgctggcggcccacggtgccccggacgctgaccccgcgcccaggggccgcagtgctgcgatgagcggccccgagcccggcagcccctaccccaacacctggcatcatcgcctgttgcagaggagcctcgtgctcttctcggttggggtggtcctagccctggtgctcaacctgctgcagatccagaggaatgtcactctcttccccgaggaggtgatcgccaccatcttttcctccgcctggtgggtccctccctgctgcgggacagcagctgctgttgttggcctactgtacccctgtatcgacagtcacctcggagaaccccacaaatttaagagagaatgggccagtgtcatgcgctgcatagcagtttttgttggcattaaccacgccagtgctaaattggattttgccaataatgtccagctgtccttgactttagcagccctatctttgggcctttggtggacatttgatcgttccagaagtggccttgggctggggatcaccatagcttttctagctacgctgatcacgcagtttctcgtgtataatggtgtctatcagtatacatccccagatttcctctatattcgttcttggctcccttgtatatttttctcaggaggcgtcacggtggggaacataggacgacagttagctatgggtgttcctgaaaagccccatagtgattgagtcttcaaaaccaccgattctgagagcaaggaagattttggaagaaaatctgactgtggattatgacaaagattatcttttttcttaagtaatctatttagatcgggctgactgtacaaatgactcctggaaaaaactcttcacctagtctagaatagggaggtggagaatgatgacttaccctgaagtcttcccttgactgcccgcactggcgcctgtctgtgccctggagcattctgcccaggctacgtgggttcaggcaggtggcagcttcccaagtattcgatttcattcatgtgattaaaacaagttgccatatttcaaagccttgaactaagactcaattaccaacccgcagttttgtgtcagtgcccaaaggaggtaggttgatggtgcttaacaaacatgaagtatggtgtaataggaataatatttatccaaaagatttttaaaaatagggctgtgtttaaaaaaaaaaacaaaacaagaaaagcagcagtgattatagagaggtcacactctaagtggggtcgcggcgtggccacgcttcacggtcacgctcgtccgtcctgcagtggcgtgtttacatggtcacacgtgtgtgtatcaccagtgggtcaactgcttgtcattcctcccgtggcagtttgtgtagacaatcttactgagcaaaaggcaatgaaaagtcttggttcccacactgcgatatattggaattttcacctcagtttatgaagtttatttcgaaatccatagtcatctaagaatgaatacctgtctgccatgtatttcaatcttagtgagccaaaattgtttgtttgttactacagaatagagatgactgttttttgccacagccctatggaatttgcaatctgtgattgccttgtaaaaaggagagtgcatatggcactgcattaaacgtgtggtgtttctagtcaatgatattggtgagcacaatgtattcatttaatggcatagaccataccagacctaatttgcaagtattgggtcttaaacttcaagtgcaatgtatatgaaaaccaatctgagccttgtatctcttaaatatttattttttttaacgtgtgagatgttcgagagaaggttctccattcatttcagtgctgcctggaggaaactcggcaatgatttctttcagttgtgaagttcctttcgtgttacaccctccactgaaccctcaaccttcgaaatactccagttttgtgggtttggtcatttttacttataaatttacctttttgtattttgcaatttacatgtgtttggtttgttttaaattctgtgaaagtggcttgattaaaagactccttttaaatggaagccaccagtcagcagaatggaagcttagaggaacttgcctgtgagcgctggtctttgtgtttggttttgtgatgtaacgatctttgctggggttttttgctttgttttgagggaaatgtcttggagtaaattttaagttcctggagttaatttgttttacaggaattttgttttttaaaaaaataggatcattctgaactttggaatgacccccttatatattttctgaaaatgaaaacagttacatgaaaaaaatttccaatgaagatgtcagcattttatgaaaaaccagaagttattagatgaaagcagcgagtgaatctttaaaacagacttgatcacgcacacacaataagtctttctctccgaaaccggaagtaaatctatatctgttagaaataatgtagccaaaagaatgtaaatttgaggatttttttgccaatagtttatagaaaatatatgaaccaaagtgatttgagtttgtaaaaatgtaaaatagtatgaacaaaatttgcactctaccagatttgaacatctagtgaggttcacattcatactaagttttcaacattgtgttctttttgcattcattttttacttttattaaaggttcaaaaccaaaaaaaaaaaa

SEQ ID NO: 12 - protein, 277 aa, Homo sapiens
MPRLHDHFWSCSCAHSARRRGPPRASAAGLAAKVGEMINVSVSGPSLLAAHGAPDADPAPRGRSAAMSGPEPGSPYPNTWHHRLLQRSLVLFSVGVVLALVLNLLQIQRNVTLFPEEVIATIFSSAWWVPPCCGTAAAVVGLLYPCIDSHLGEPHKFKREWASVMRCIAVFVGINHASAKLDFANNVQLSLTLAALSLGLWWTFDRSRSGLGLGITIAFLATLITQFLVYNGVYQYTSPDFLYIRSWLPCIFFSGGVTVGNIGRQLAMGVPEKPHSD

SEQ ID NO: 13 - DNA, 5473 bp, Homo sapiens
  CDS (491)..(1570)
ggcaggacgaggtggcaccaaattcccttcggccaatgacgagccggagtttacagaagcctcattagcatttccccagaggcaggggcaggggcagaggccgggtggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatagaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagttttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagattgtgtttgttcataataaaagtgaagtgaatctgattgcaaaaaa

SEQ ID NO: 14 - protein, 359 aa, Homo sapiens
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 15 - DNA, 1689 bp, Homo sapiens
  CDS (146)..(1267)
aggaaatgaggtgagaagtacgtccactcttctgtccagcttttaacaatctaactaatgccctctccagggtcaccctagaatcagatctgctccccagcatcttctgtttcctggtgagtgattcctgctactttggatggccatgacgggctggagctgccttgtgacaggagcaggagggtttctgggacagaggatcatccgcctcttggtgaaggagaaggagctgaaggagatcagggtcttggacaaggccttcggaccagaattgagagaggaattttctaaactccagaacaagaccaagctgacagtgctggaaggagacattctggatgagccattcctgaagagagcctgccaggacgtctcggtcatcatccacaccgcctgtatcattgatgtcttcggtgtcactcacagagagtctatcatgaatgtcaatgtgaaaggtacccagctcctgttagaggcctgtgtccaagctagtgtgccagtcttcatctacaccagtagcatagaggtagccgggcccaactcctacaaggaaatcatccagaatggccatgaagaagagcctctggaaaacacatggcccgctccatacccacacagcaaaaagcttgctgagaaggctgtactggcggctaacgggtggaatctgaaaaacggcggcaccctgtacacttgtgccttacgacccatgtatatctatggggaaggaagccgattcctttctgctagtataaacgaggccctgaacaacaatgggatcctgtcaagtgttggaaagttctccactgttaacccagtctatgttggcaatgtggcctgggcccacattctggccttgagggccctgcaggaccccaagaaggccccaagcatccgaggacagttctactatatctcagatgacacgcctcaccaaagctatgataaccttaattacaccctgagcaaagagttcggcctccgccttgattccagatggagctttcctttatccctgatgtattggattggcttcctgctggaaatagtgagcttcctactcaggccaatttacacctatcgaccgcccttcaaccgccacatagtcacattgtcaaatagcgtattcaccttctcttataagaaggctcagcgagatctggcgtataagccactctacagctgggaggaagccaagcagaaaacggtggagtgggttggttcccttgtggaccggcacaaggagaccctgaagtccaagactcagtgatttaaggatgacagagatgtgcatgtgggtattgttaggagatgtcatcaagctccaccctcctggcctcatacagaaagtgacaagggcacaagctcaggtcctgctgcctccctttcatacaatggccaacttattgtattcctcatgtcatcaaaacctgcgcagtcattggcccaacaagaaggtttctgtcctaatcatataccagaggaaagaccatgtggtttgctgttaccaaatctcagtagctgattctgaacaatttagggactcttttaacttgagggtcgttttgactactagagctccatttctactcttaaatgagaaaggatttcctttctttttaatcttccattccttcacatagtttgataaaaagatcaataaatgtttgaatgtttaatgtgaaaaaaaaa

SEQ ID NO: 16 - protein, 373 aa, Homo sapiens
MTGWSCLVTGAGGFLGQRIIRLLVKEKELKEIRVLDKAFGPELREEFSKLQNKTKLTVLEGDILDEPFLKRACQDVSVIIHTACIIDVFGVTHRESIMNVNVKGTQLLLEACVQASVPVFIYTSSIEVAGPNSYKEIIQNGHEEEPLENTWPAPYPHSKKLAEKAVLAANGWNLKNGGTLYTCALRPMYIYGEGSRFLSASINEALNNNGILSSVGKFSTVNPVYVGNVAWAHILALRALQDPKKAPSIRGQFYYISDDTPHQSYDNLNYTLSKEFGLRLDSRWSFPLSLMYWIGFLLEIVSFLLRPIYTYRPPFNRHIVTLSNSVFTFSYKKAQRDLAYKPLYSWEEAKQKTVEWVGSLVDRHKETLKSKTQ

SEQ ID NO: 17 - DNA, 1753 bp, Homo sapiens
  CDS (128)..(1537)
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 18 - protein, 469 aa, Homo sapiens
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 19 - DNA, 5623 bp, Homo sapiens
  CDS (1)..(3744)
atgtcactttttttaagggtagtatttagtttcacaatgtttggggacctgtttgaagaggagtattccactgtgtctaataatcagtatggaaaagggaagaaattaaagactaaagctttggagccacctgctcctagagaattcaccaatttaagcggaatcagaaatcagggtggaacctgttacctcaattcccttcttcagactcttcatttcacacctgaattcagagaagctctattttctcttggcccagaagagcttggtttgtttgaagataaggataaacccgatgcaaaggttcgaatcatccctttacagttacagcgcttgtttgctcagcttctgctcttagaccaggaagctgcatccacagcagacctcactgacagctttgggtggaccagtaatgaggaaatgaggcaacatgatgtgcaggaactgaatcgaatcctcttcagcgctttggaaacttctttagttgggacctccggtcatgacctcatctatcgtctgtaccatggaaccattgttaaccagattgtttgtaaagaatgtaagaacgttagcgagaggcaggaagacttcttagatctaacagtagcagtcaaaaatgtatccggtttggaagatgctctctggaacatgtatgtagaagaggaagtttttgattgtgacaacttgtaccactgtggaacttgtgacaggctggttaaagcagcaaagtcggccaaattacgtaagctgcctccttttcttactgtttcattactaagatttaattttgattttgtgaaatgcgaacgctacaaggaaactagctgttatacattccctctccggattaatctcaagcccttttgtgaacagagtgaattggatgacttagaatatatatatgacctcttctcagttattatacacaaaggtggctgctacggaggccattaccatgtatatattaaagatgttgatcatttgggaaactggcagtttcaagaggaaaaaagtaaaccagatgtgaatctgaaagatctccagagtgaagaagagattgatcatccactgatgattctaaaagcaatcttattagaggagaataatctaattcctgttgatcagctgggccagaaacttttgaaaaagataggaatatcttggaacaagaagtacagaaaacagcatggaccactgcggaagttcttacagctccattctcagatatttctactcagttcagatgaaagtacagttcgtctcttgaagaatagttctctccaggctgagtctgatttccaaaggaatgaccagcaaattttcaagatgcttcctccagaatccccaggtttaaacaatagcatctcctgtccccactggtttgatataaatgattctaaagtccagccaatcagggaaaaggatattgaacagcaatttcagggtaaagaaagtgcctacatgttgttttatcggaaatcccagttgcagagaccccctgaagctcgagctaatccaagatatggggttccatgtcatttactgaatgaaatggatgcagctaacattgaactgcaaaccaaaagggcagaatgtgattctgcaaacaatacttttgaattgcatcttcacctgggccctcagtatcatttcttcaatggggctctgcacccagtagtctctcaaacagaaagcgtgtgggatttgacctttgataaaagaaaaactttaggagatctccggcagtcaatatttcagctgttagaattttgggaaggagacatggttcttagtgttgcaaagcttgtaccagcaggacttcacatttaccagtcacttggcggggatgaactgacactgtgtgaaactgaaattgctgatggggaagacatctttgtgtggaatggggtggaggttggtggagtccacattcaaactggtattgactgcgaacctctacttttaaatgttcttcatctagacacaagcagtgatggagaaaagtgttgtcaggtgatagaatctccacatgtctttccagctaatgcagaagtgggcactgtcctcacagccttagcaatcccagcaggtgtcatcttcatcaacagtgctggatgtccaggtggggagggttggacggccatccccaaggaagacatgaggaagacgttcagggagcaagggctcagaaatggaagctcaattttaattcaggattctcatgatgataacagcttgttgaccaaggaagagaaatgggtcactagtatgaatgagattgactggctccacgttaaaaatttatgccagttagaatctgaagagaagcaagttaaaatatcagcaactgttaacacaatggtgtttgatattcgaattaaagccataaaggaattaaaattaatgaaggaactagctgacaacagctgtttgagacctattgatagaaatgggaagcttctttgtccagtgccggacagctatactttgaaggaagcagaattgaagatgggaagttcattgggactgtgtcttggaaaagcaccaagttcgtctcagttgttcctgttttttgcaatggggagtgacgttcaacctgggacagaaatggaaatcgtagtagaagaaacaatatctgtgagagattgtttaaagttaatgctgaagaaatctggcctacaaggagatgcctggcatttacgaaaaatggattggtgctatgaagctggagagcctttatgtgaagaagatgcaacactgaaagaacttctgatatgttctggagatactttgcttttaattgaaggacaacttcctcctctgggtttcctgaaggtgcccatctggtggtaccagcttcagggtccctcaggacactgggagagtcatcaggaccagaccaactgtacttcgtcttggggcagagtttggagagccacttccagccaaggtgcttctgggaacgagcctgcgcaagtttctctcctctacttgggagacatagagatctcagaagatgccacgctggcggagctgaagtctcaggccatgaccttgcctcctttcctggagttcggtgtcccgtccccagcccacctcagagcctggacggtggagaggaagcgcccaggcaggcttttacgaactgaccggcagccactcagggaatataaactaggacggagaattgagatctgcttagagccccttcagaaaggcgaaaacttgggcccccaggacgtgctgctgaggacacaggtgcgcatccctggtgagaggacctatgcccctgccctggacctggtgtggaacgcggcccagggtgggactgccggctccctgaggcagagagttgccgatttctatcgtcttcccgtggagaagattgaaattgccaaatactttcccgaaaagttcgagtggcttccgatatctagctggaaccaacaaataaccaagaggaaaaagaaaaaaaaacaagattatttgcaaggggcaccgtattacttgaaagacggagatactattggtgttaagaatctcctgattgacgacgatgatgatttcagtacaatcagagatgacactggaaaagaaaagcagaaacaacgggccctggggagaaggaaaagccaagaagccctccatgagcagagcagctacatcctctccagtgcagagacgcctgcccggccccgagccccggaaacttctctctccatccacgtggggagcttcagataaccgcgccgctgcacggctctactcccgatgaactctccggctgatgccacaaacgtgggtttcctgggcatggggactggctgcctggcgcctccaatcccaaatcctctgcttcctttgagcacagggacggctcctctgaggcctggccagtgcatgtagtcacttagctctgcaacacgtggcagccacgggggctggtgcagctctggatgtcgcccacccagctgccagtaggtgctgggctctctcacacagcacccggccccagctgcctttttttttcttttaaccagaaaatgcacaacgtgtgcgtgaaccgcaggtatggaggcagcggcatgccgttgctccgctgtgggaggtgtgtggggtcaggccagccactttcctccgtgttcagatgactctcgttcgccctgaccggcttctcacagtgtctcaggccactgcgccaccgcgctggtgctgagcagaagcgggcagaagtggggtctgctttcaggacttcatttcccccactcgttccggccccgcatgctccacgtctgccctttggtctgagttaaaactgcgatgctgaaaagtgcgagctctttccacgaggaggagccacacagggtggcctccgagggtgagtcgctctgctaagcaagggcagccgctgcacgtcagcccgcaggccaagggtccagcttatcctgggtgctctgtgatcagaaggtccttggatcccgaggactgcagctgctgccgtccacagcgcctcagcctctcttcctgtgtggaagcggggcaggcagggctgtggcagacaggcctggcatagcccaggttccaggtgtcctgggcagcctgaccggtttcctgagtagcctcaatgaaaacacctgagaaaaagaaaatgtaaactaaggaagggttttcccacttaatccaacccagatttctattcaacttaaaggatttaattggcatttgtgagaaataatagtacctgtgtcactgagttgattgatttgggacatagacggttgactttcccagacagcagcttagcggaaagcagcatcccgaagacggtgtcgcatgtcctgagcctgcttgtcacctgggcctcacggtttctcgtcagccccgtcactgatgggaagcagcactgaagtgtgagggcagaaaggacaccgctggagtcaggcgctcccctgcctgtgacccttttccaacaagctgatgctgagccagcggcctgctttgtccctcatactcctcccaacagtccaaacccctaccagacagaagccacgtcgcttctgcacacgtgtctgagagcgtctgccatagacgtctgtgcgtttccgtgaagcactgcgccctaaggacccaactctgactagcagctgcctcccactcccaaacactaaccccgctcaggagactcgcactgggtgtagactgcgtctttgcagagggagatgatagaggcttctgatgacgccagagctgtaagtgtcgtgacgagctgggcgcccagcccttcttaagctgttctgtttctgtggctttaactgacatatttctgtagcatctgccttcatctcatctcagcgtaatgaaatattaatgaaatcgctgaaaagctttgccttcgagaggccagaagcctcgcggaatgtctgcaagtccaaagacgcgtgtgggttgtgccctgaagtgccgtccagcaggcgcgtgcggccgggccggcctgtgcgtgtggcctttgccttcttccctttcttcctgttttctgtttttttaatttggggattgagaaagctgtacgattttgttaaagaaaaaaataaaccattttttaaagttgtagcccttaaaaaaa

SEQ ID NO: 20 - protein, 1247 aa, Homo sapiens
MSLFLRVVFSFTMFGDLFEEEYSTVSNNQYGKGKKLKTKALEPPAPREFTNLSGIRNQGGTCYLNSLLQTLHFTPEFREALFSLGPEELGLFEDKDKPDAKVRIIPLQLQRLFAQLLLLDQEAASTADLTDSFGWTSNEEMRQHDVQELNRILFSALETSLVGTSGHDLIYRLYHGTIVNQIVCKECKNVSERQEDFLDLTVAVKNVSGLEDALWNMYVEEEVFDCDNLYHCGTCDRLVKAAKSAKLRKLPPFLTVSLLRFNFDFVKCERYKETSCYTFPLRINLKPFCEQSELDDLEYIYDLFSVIIHKGGCYGGHYHVYIKDVDHLGNWQFQEEKSKPDVNLKDLQSEEEIDHPLMILKAILLEENNLIPVDQLGQKLLKKIGISWNKKYRKQHGPLRKFLQLHSQIFLLSSDESTVRLLKNSSLQAESDFQRNDQQIFKMLPPESPGLNNSISCPHWFDINDSKVQPIREKDIEQQFQGKESAYMLFYRKSQLQRPPEARANPRYGVPCHLLNEMDAANIELQTKRAECDSANNTFELHLHLGPQYHFFNGALHPVVSQTESVWDLTFDKRKTLGDLRQSIFQLLEFWEGDMVLSVAKLVPAGLHIYQSLGGDELTLCETEIADGEDIFVWNGVEVGGVHIQTGIDCEPLLLNVLHLDTSSDGEKCCQVIESPHVFPANAEVGTVLTALAIPAGVIFINSAGCPGGEGWTAIPKEDMRKTFREQGLRNGSSILIQDSHDDNSLLTKEEKWVTSMNEIDWLHVKNLCQLESEEKQVKISATVNTMVFDIRIKAIKELKLMKELADNSCLRPIDRNGKLLCPVPDSYTLKEAELKMGSSLGLCLGKAPSSSQLFLFFAMGSDVQPGTEMEIVVEETISVRDCLKLMLKKSGLQGDAWHLRKMDWCYEAGEPLCEEDATLKELLICSGDTLLLIEGQLPPLGFLKVPIWWYQLQGPSGHWESHQDQTNCTSSWGRVWRATSSQGASGNEPAQVSLLYLGDIEISEDATLAELKSQAMTLPPFLEFGVPSPAHLRAWTVERKRPGRLLRTDRQPLREYKLGRRIEICLEPLQKGENLGPQDVLLRTQVRIPGERTYAPALDLVWNAAQGGTAGSLRQRVADFYRLPVEKIEIAKYFPEKFEWLPISSWNQQITKRKKKKKQDYLQGAPYYLKDGDTIGVKNLLIDDDDDFSTIRDDTGKEKQKQRALGRRKSQEALHEQSSYILSSAETPARPRAPETSLSIHVGSFR

SEQ ID NO: 21 - DNA, 1805 bp, Homo sapiens
  CDS (114)..(998)
caaatgcagaagtggttctcatctttttttgcagcttaagatctgccttggtatttgaagagatataaactagatcaatttctttcacaggatcaactaaacagtgtaccacaatgaattctgaacttgactattatgaaaagtttgaagaagtccatgggattctaatgtataaagattttgtcaaatattgggataatgtggaagcgttccaggcaagaccagatgatcttgtcattgccacctaccctaaatctggtacaacctgggttagtgaaattgtgtatatgatctataaagagggtgatgtggaaaagtgcaaagaagatgtaatttttaatcgaatacctttcctggaatgcagaaaagaaaacctcatgaatggagtaaaacaattagatgagatgaattctcctagaattgtgaagactcatttgccacctgaacttcttcctgcctcattttgggaaaaggattgtaagataatctatctttgccggaatgcaaaggatgtggctgtttccttttattatttctttctaatggtggctggtcatccaaatcctggatcctttccagagtttgtggagaaattcatgcaaggacaggttccttatggttcctggtataaacatgtaaaatcttggtgggaaaagggaaagagtccacgtgtactatttcttttctacgaagacctgaaagaggatatcagaaaagaggtgataaaattgatacatttcctggaaaggaagccatcagaggagcttgtggacaggattatacatcatacttcgttccaagagatgaagaacaatccatccacaaattacacaacactgccagacgaaattatgaaccagaaattgtcgcccttcatgagaaagggaattacaggagactggaaaaatcactttacagtagccctgaatgaaaaatttgataaacattatgagcagcaaatgaaggaatctacactgaagtttcgaactgagatctaagaaggtctttctttacttaacatatctgatattaaagatttcttttcattattctccactttttcttattttagattgctagaaaagacataatcatggattatgttgacattttctttttaaatttttgtttaactttttttttttttttttgagacagagtctcactctgttgcctaggctggaggacagtggcacaatcatggctgattgcagccttgacctccttgactcaattgatcctcccatctcagcctcccaagtagctaggactacagacatgtgcaaccatgtttggctaatttttttaatgtttttttgtagagatgaggtcttattatattgtccaggctggtcttgaattcctgggctcaagcttcccaagtagctgcaacaacaggcacacaccaccatgctcaactaattttatttctattttttgtatagacaggggcttgctatagtgtccaggctggtctgaaacccttgagctcaagtgatcttcccacaccagcctcccaaaatactgggattacaggcttgagcctccatgcctggcccaggtaacatgtttattgagctgtacatgcatatgagaaataagaaacttttttttcctactatcatctcttaaattttgttttctttttcttttgcttcctcttcttcttttctattttttataaatatcatgcacaactataacctatgggaatgatgtagtaacacagattattcatcttgttagagttgtattaaaaataaacaagcatttcaaattaaaaaaaaaaaaaaaaaaaaaaaaaa

SEQ ID NO: 22 - protein, 294 aa, Homo sapiens
MNSELDYYEKFEEVHGILMYKDFVKYWDNVEAFQARPDDLVIATYPKSGTTWVSEIVYMIYKEGDVEKCKEDVIFNRIPFLECRKENLMNGVKQLDEMNSPRIVKTHLPPELLPASFWEKDCKIIYLCRNAKDVAVSFYYFFLMVAGHPNPGSFPEFVEKFMQGQVPYGSWYKHVKSWWEKGKSPRVLFLFYEDLKEDIRKEVIKLIHFLERKPSEELVDRIIHHTSFQEMKNNPSTNYTTLPDEIMNQKLSPFMRKGITGDWKNHFTVALNEKFDKHYEQQMKESTLKFRTEI

SEQ ID NO: 23 - DNA, 20 bp, Artificial
  PCR primer
cccctgagctgatgtttgat

SEQ ID NO: 24 - DNA, 20 bp, Artificial
  PCR primer
gacgcccagattcaaaggta

SEQ ID NO: 25 - DNA, 20 bp, Artificial
  PCR primer
gcagacacacctgttggaaa

SEQ ID NO: 26 - DNA, 20 bp, Artificial
  PCR primer
gtcacggggatttctttgaa

SEQ ID NO: 27 - DNA, 20 bp, Artificial
  PCR primer
gtcaccgtgtcaacctgatg

SEQ ID NO: 28 - DNA, 20 bp, Artificial
  PCR primer
gcctgccttcaagatttctg

SEQ ID NO: 29 - DNA, 20 bp, Artificial
  PCR primer
ggctgaggtcactacggaaa

SEQ ID NO: 30 - DNA, 20 bp, Artificial
  PCR primer
ggtctcagattgggcactgt

SEQ ID NO: 31 - DNA, 22 bp, Artificial
  PCR primer
gtgctgccctcagactatgacc

SEQ ID NO: 32 - DNA, 23 bp, Artificial
  PCR primer
ctgccatacagaacatcgctcag

SEQ ID NO: 33 - DNA, 20 bp, Artificial
  PCR primer
ggtggacatttgatcgttcc

SEQ ID NO: 34 - DNA, 20 bp, Artificial
  PCR primer
tgacgcctcctgagaaaaat

SEQ ID NO: 35 - DNA, 20 bp, Artificial
  PCR primer
tggtttcacttggagctgtg

SEQ ID NO: 36 - DNA, 20 bp, Artificial
  PCR primer
gccatgcaatcaatgaagaa

SEQ ID NO: 37 - DNA, 20 bp, Artificial
  PCR primer
aggacgtctcggtcatcatc

SEQ ID NO: 38 - DNA, 20 bp, Artificial
  PCR primer
ctggcacactagcttggaca

SEQ ID NO: 39 - DNA, 20 bp, Artificial
  PCR primer
caggaactcatgagcgtgaa

SEQ ID NO: 40 - DNA, 20 bp, Artificial
  PCR primer
gatattcacggctcccactc

SEQ ID NO: 41 - DNA, 20 bp, Artificial
  PCR primer
ggggcaccgtattacttgaa

SEQ ID NO: 42 - DNA, 20 bp, Artificial
  PCR primer
ggcttcttggcttttccttc

SEQ ID NO: 43 - DNA, 20 bp, Artificial
  PCR primer
cagaaattgtcgcccttcat

SEQ ID NO: 44 - DNA, 21 bp, Artificial
  PCR primer
gattccttcatttgctgctca

SEQ ID NO: 45 - DNA, 1567 bp, Homo sapiens
  CDS (132)..(1325)
ggtgcgcggggaggtggagggcgaggggcggggctacctcaggtcccgcccgcggcaggcctgtgggctgcgaggaggagctttgcctagcttgcaggcagcgcagggcagacggcggcaggagaagcaagatgaatgcaggctcagatcctgtggtcatcgtctcggcggcgcggaccatcataggttccttcaatggtgccttagctgctgttcctgtccaggacctgggctccactgtcatcaaagaagtcttgaagagggccactgtggctccggaagatgtgtctgaggtcatctttggacatgtcttggcagcaggctgtgggcagaatcctgttagacaagccagtgtgggtgcaggaattccctactctgttccagcatggagctgccagatgatctgtgggtcaggcctaaaagctgtgtgccttgcagtccagtcaatagggataggagactccagcattgtggttgcaggaggcatggaaaatatgagcaaggctcctcacttggcttacttgagaacaggagtaaagataggtgagatgccactgactgacagtatactctgtgatggtcttacagatgcatttcacaactgtcatatgggtattacagctgaaaatgtagccaaaaaatggcaagtgagtagagaagatcaggacaaggttgcagttctgtcccagaacaggacagagaatgcacagaaagctggccattttgacaaagagattgtaccagttttggtgtcaactagaaaaggtcttattgaagttaaaacagatgagtttcctcgccatgggagcaacatagaagccatgtccaagctaaagccttactttcttactgatggaacgggaacagtcaccccagccaatgcttcaggaataaatgatggtgctgcagctgtcgttcttatgaagaagtcagaagctgataaacgtgggcttacacctttagcacggatagtttcctggtcccaagtgggtgtggagccttccattatgggaataggaccaattccagccataaagcaagctgttacaaaagcaggttggtcactggaagatgttgacatatttgaaatcaatgaagcctttgcagctgtctctgctgcaatagttaaagaacttggattaaacccagagaaggtcaatattgaaggaggggctatagccttgggccaccctcttggagcatctggctgtcgaattcttgtgaccctgttacacacactggagagaatgggcagaagtcgtggtgttgcagccctgtgcattgggggtgggatgggaatagcaatgtgtgttcagagagaatgaattgcttaaactttgaacaacctcaatttctttttaaactaataaagtactaggttgcaatatgtgaaatcagaggaccaaagtacagatggaaaccatttcctacatcacaaaaacccaagtttacagcttgtactttactttaatgtgtaatactcaactcaaggtacaagacaattgcatttaacattgttataaataaaaggaacatcagatcaatcattaaaaaaaaaaaaaaaaaa

SEQ ID NO: 46 - protein, 397 aa, Homo sapiens
MNAGSDPVVIVSAARTIIGSFNGALAAVPVQDLGSTVIKEVLKRATVAPEDVSEVIFGHVLAAGCGQNPVRQASVGAGIPYSVPAWSCQMICGSGLKAVCLAVQSIGIGDSSIVVAGGMENMSKAPHLAYLRTGVKIGEMPLTDSILCDGLTDAFHNCHMGITAENVAKKWQVSREDQDKVAVLSQNRTENAQKAGHFDKEIVPVLVSTRKGLIEVKTDEFPRHGSNIEAMSKLKPYFLTDGTGTVTPANASGINDGAAAVVLMKKSEADKRGLTPLARIVSWSQVGVEPSIMGIGPIPAIKQAVTKAGWSLEDVDIFEINEAFAAVSAAIVKELGLNPEKVNIEGGAIALGHPLGASGCRILVTLLHTLERMGRSRGVAALCIGGGMGIAMCVQRE

SEQ ID NO: 47 - DNA, 3105 bp, Homo sapiens
  CDS (179)..(1720)
caactgggggcgccccggacgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcaatgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttcccttgtagaaagtgaggctcagattaaattttagaatattttctaaatgtctttttcacaatcatgtactgggaaggcaatttcatactaaactgattaaataatacatttataatctacaactgtttgcacttacagctttttttgtaaatataaactataatttattgtctattttatatctgttttgctgtaacattgaaggaaagaccagacttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggca

SEQ ID NO: 48 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 49 - DNA, 3691 bp, Homo sapiens
  CDS (109)..(981)
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 50 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 51 - DNA, 2358 bp, Homo sapiens
  CDS (207)..(1256)
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 52 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 53 - DNA, 5742 bp, Homo sapiens
  CDS (237)..(2972)
gttttagcggggactacgatccaggctggagttgcgctcggccggtctgagcgctggcgctgcccggacgccgcggggtccccgccagcccagggcactcggcgcggggatctgcgcgcctcgctctcccttcccgatgccgccgcccggctgctgatcgccgcaccaccttccctcatcggcttgggtccgtggaggtccctgcagaggcaggaagcctccttaggaaagcagggatggaggtaaattgtttaacactaaaagacctgatcagccccaggcagcccagactagattttgcagttgaagatggggaaaatgcacaaaaggaaaatatatttgttgatcgatcaaggatggccccgaagactccaataaaaaatgaaccaattgatttatcgaagcaaaaaaaatttactccagaaagaaatcccattactccagttaagtttgttgacagacagcaagcggaaccatggacacccacagctaacctgaagatgctcattagtgctgccagcccagatataagggaccgggagaagaaaaagggactattccgacccattgaaaacaaggacgatgcatttacagattctctacagcttgatgttgttggggacagtgctgtggacgaatttgaaaagcaaaggccaagcagaaaacagaaaagtttaggactcctgtgccagaagtttctagctcgctatccaagttatcccttgtcaactgagaaaactaccatctccctagatgaagttgctgtcagtcttggtgtggaaaggagacgcatctatgacattgtaaatgtgctggagtcgctgcatctggtcagccgggtggctaagaatcagtatggctggcatggacggcacagcctgccaaaaaccctgaggaacctccagagactaggagaggagcagaaatatgaagagcaaatggcctacctccaacagaaagagctggacctgatagattataaatttggagaacgtaaaaaagatggtgatccagattcccaggaacaacagttactggatttctctgaacccgactgtccctcttcatctgcaaacagtagaaaagacaagtctctgagaattatgagccagaagtttgtcatgctgttcctcgtctccaaaaccaagattgtcactctggatgtggctgccaaaatactgatagaagaaagccaagatgccccagaccatagtaaatttaaaacaaaggtacgacgcctctatgacatagccaatgttctgaccagcttggctctgataaagaaagtgcatgtaacagaagagcgaggtcgtaaaccagccttcaagtggatcgggcctgtggacttcagctcaagtgatgaagaactggtggatgtttctgcatctgtcttaccagaattgaaaagagaaacatatggccagattcaagtctgtgcaaaacagaagctggctcgccatggttcttttaacacagttcaggcttctgagaggatccagaggaaagtgaactcagaaccgagcagcccgtacagagaagaacaaggatcaggtggctactctttagaaattggaagcctggcagctgtctatagacagaaaatagaagacaattcacagggaaaagcctttgccagtaagagagtggtgcctccatcaagcagcttggaccctgttgctcctttccctgtcctctctgttgacccagaatattgtgttaatcctttagcccacccagtattttctgttgctcagacggacctgcaggcattctccatgcagaacggtctgaatggacaagtggatgtctcacttgcttctgcagcctctgctgtggagagcctgaagccagcactccttgctggccagcctctagtgtatgtgccctctgcctcactgttcatgctgtatggaagtctgcaggagggaccagcgtcagggtcagggtcagagagggatgacagaagctcagaagccccagccacagtagagctgtcatctgcaccctcagctcagaagcgcctctgtgaggagaggaaacctcaggaggaggatgagccagccactaaaaggcaaagtagggaatatgaagacggcccgctgtcgcttgtcatgcccaagaaaccctcagattccacagaccttgcctctcccaagactatgggtaacagggcatctatacccctcaaagacattcatgtgaatggccaactccctgctgcagaagagatttcaggaaaggcaacagcaaactctcttgtttcttctgagtggggaaatccttcaagaaatacagatgttgaaaagccttcaaaagaaaatgaaagcaccaaagagccttctttgctacaatatctttgtgtgcagtctcctgcaggattaaatggtttcaatgtacttttatctggcagtcaaaccccccctactgtgggcccgtcctcaggtcagctgccgtctttcagtgtcccttgcatggtcttaccatctccacctctgggcccttttcctgttctctattctcctgcaatgccgggcccggtttcttctactcttggtgctctcccaaacacaggacctgtgaatttcagcttgcctggccttggatcaatagcccagcttctcgtcggccccacagctgtggttaatccaaagtcgtccacactcccttctgcagaccctcagcttcagagtcagccctcactaaacctaagtccagtgatgtcaaggtcacacagtgtcgtccaacaacctgagtcccccgtttacgtgggacatccagtctcagtagtaaaattacatcagtcaccagttccagtgacccccaagagcatccaacgcacacatcgtgagacgtttttcaagacacccggcagccttggagaccctgtcctgaagagaagagaaaggaaccagtcacgaaacaccagctcggcccagaggagactagaaatccccagcggcggcgctgactaacctgccgctttgccaggtgggggtgggatcaaacgccctgagagtcccggatgtccgaggcgggatgcaaaccatcccgtcctgagcacgggtccttcctctctctttcatccacacttctgttaacttcccaccaccatcaatcatctgatttcctgaaagtaattaattgtgcatttaataccagttagagttccgactctgcatggtgtcacagtgaaagcgccgactgacttatggttttgattcaagaatcgtcttattctggaagtagatctgaataggctaccggagccttgtttttctaaaggggggcgctgtctagcacttaactagggtaagcattcttaacatgtatttccacttgccctgagtaaatctgtggtgagagaagcttcctttctgcagtttaaaaaagctactgcttccttaggcttcatcaggaagccaccttcagttgtgaatcctatggtgttatttattttgttcctgaaatgggatttagtgcaaaaagtttacaactacagtctttaacacatttttttcagggtatgacgacttgaatgtttatacttttattctataatttgccctgcacttattttacaacctagtaataatgtggataaatgtatctacatgacacatgtcaagaccaaaataactgtgaatgacacaccttgctgtaaatgaactgtgctaaccctgactgtgggcttgagaacaaagatgaactctagaactctagcagcctaactgctgcttctcaaataactgtgtgaacagtgagatattactgtttgtttctaaaaatcctactgtgcccagtttccttcactacatgccctgcattttttatttaaatatttagctgtagcgccatcagatatggatgccttctaacaattgctgtttgtaaaataaatcaggatggtagaaagtgattatatggaaaattggaacctggatgagaccttttcgttgaattctgaagagtaatgatgtgaaaattgatacagggcaagagatgattcttttgtttttcttctacttcatgtccagaagagtaagagggaaaatggacatatgtttcatatccaagggtattcaaactgtagttagttggtacctctgaaaaatgagaatggtgagcgcacgggttggttgttctagcatgaatacaattctggaaactgttatgcaatttcccttttttaacccacattactttaggggtgcattaagtcgccaaactatactagttctttgtattcctagacttgctgatatttacctctctcttgtctcttcagagtaaatggttcccttctttccttcctactttccttcattctctcttccttccctccttcctacttcttttcttccttcctcttcctctcttaaaactatcttagatgtagaatcctggtgtagggttttattttatttttattttttgacccaataaaatgttatatgaaagaatgaaaatattaatttaagagactctgggagtctgaataaagtagctttatattaactacaggataatattagccttattacccccacaagattttttaaaacttgaggtaggtagctacattaaataaatttgctacttatataaaaatttttatcaacactaaacttttaaagtttacaagttttttttttcttttttacagtcttctatagagttaggttaaaaatgtggttctaaccatcaacaattgcatggttaaatgaccctgaactaaaactgatgggttccctatcaaaacaaataaaaatatacctttttcaggtttcaatctgtgcagggtatatgcatgttaattctaccatgcttaagaacttccacaaaatatttcatggagaggtctgcatttagacggaaacagaaattgcttttcccctcactgttcctgaatgctctatacttgttttaacatttttgctatctttttttattattctgatcatgatatgaccatttaacctcagaattcataattcctgaggggtgttaagaagcagtcccattggtgaggatattatgacttggtgaccattcttaggagtagaaaaccaaggacaattgcttctgtattcagtatccacttcttaatgtggctttatatgtaaaaataataatgcagtggttgtttctgtcaggaaaataaatcttacagaacaactggtggaattgaagctgctgcgctagacttggatattttgggtagtgaagaagcaatggcaatcttgagtctattattgtataatttagtaaaagaaaaaaataatcgttggtggtcctactaagagaatgcagcttttttgagttgtcacagaggctgtgtgtgccctacactgaccagggtttgtaaaaccctttcattctggtacaagagtcgggggtataacttttatacttgaatctacctaccaagtttacatttctcaattcctttttgtaaggtgctatttctgtatttaaataactttcttttaacgtaaagctgctttctgcttatcttattgcactgctagttgtatgtaggtattaattttattgctgcttactgcttttgttttcttattatttagctctgctctttttcctaatggctatattatctatagctatttacttgtaactgtactacatgtaaactgattttttgttctgattttttttctaatatttttaggaaaatattaagctttataaaatagcaataaaaaataattcatttaagaagaa

SEQ ID NO: 54 - protein, 911 aa, Homo sapiens
MEVNCLTLKDLISPRQPRLDFAVEDGENAQKENIFVDRSRMAPKTPIKNEPIDLSKQKKFTPERNPITPVKFVDRQQAEPWTPTANLKMLISAASPDIRDREKKKGLFRPIENKDDAFTDSLQLDVVGDSAVDEFEKQRPSRKQKSLGLLCQKFLARYPSYPLSTEKTTISLDEVAVSLGVERRRIYDIVNVLESLHLVSRVAKNQYGWHGRHSLPKTLRNLQRLGEEQKYEEQMAYLQQKELDLIDYKFGERKKDGDPDSQEQQLLDFSEPDCPSSSANSRKDKSLRIMSQKFVMLFLVSKTKIVTLDVAAKILIEESQDAPDHSKFKTKVRRLYDIANVLTSLALIKKVHVTEERGRKPAFKWIGPVDFSSSDEELVDVSASVLPELKRETYGQIQVCAKQKLARHGSFNTVQASERIQRKVNSEPSSPYREEQGSGGYSLEIGSLAAVYRQKIEDNSQGKAFASKRVVPPSSSLDPVAPFPVLSVDPEYCVNPLAHPVFSVAQTDLQAFSMQNGLNGQVDVSLASAASAVESLKPALLAGQPLVYVPSASLFMLYGSLQEGPASGSGSERDDRSSEAPATVELSSAPSAQKRLCEERKPQEEDEPATKRQSREYEDGPLSLVMPKKPSDSTDLASPKTMGNRASIPLKDIHVNGQLPAAEEISGKATANSLVSSEWGNPSRNTDVEKPSKENESTKEPSLLQYLCVQSPAGLNGFNVLLSGSQTPPTVGPSSGQLPSFSVPCMVLPSPPLGPFPVLYSPAMPGPVSSTLGALPNTGPVNFSLPGLGSIAQLLVGPTAVVNPKSSTLPSADPQLQSQPSLNLSPVMSRSHSVVQQPESPVYVGHPVSVVKLHQSPVPVTPKSIQRTHRETFFKTPGSLGDPVLKRRERNQSRNTSSAQRRLEIPSGGAD

SEQ ID NO: 55 - DNA, 2112 bp, Homo sapiens
  CDS (335)..(973)
ggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagcacgttgttccgtatggacttggaagccctaggtccaagagagccttggagaatttacttcccacaaaggcaacagaccgtgaaaatagatgccaatgtgctagccaaaaagacaagaagtgctggaatttttgccaagcaggaaaagaactcagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtcggagaccatgagaaacagcgtcaaatcatcttttcatgatcccaagctgaaaggcaagccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaagaaagacttttgtttaaatttgtaaaatgcaaaactgaatgaaactgttactaccataaatcaggatatgtttcatgaatatgagtctacctcacctatattgcactctggcagaagtatttcccacatttaattattgcctccccaaactcttcccacccctgctgccccttcctccatcccccatactaaatcctagcctcgtagaagtctggtctaatgtgtcagcagtagatataatattttcatggtaatctactagctctgatccataagaaaaaaaagatcattaaatcaggagattccctgtccttgatttttggagacacaatggtatagggttgtttatgaaatatattgaaaagtaagtgtttgttacgctttaaagcagtaaaattattttcctttatataaccggctaatgaaagaggttggattgaattttgatgtacttatttttttatagatatttatattcaaacaatttattccttatatttaccatgttaaatatctgtttgggcaggccatattggtctatgtatttttaaaatatgtatttctaaatgaaattgagaacatgctttgttttgcctgtcaaggtaatgactttagaaaataaatatttttttccttactgtaaaaaaaaaaaaa

SEQ ID NO: 56 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 57 - DNA, 3182 bp, Homo sapiens
  CDS (935)..(2206)
ggcaggcgagccggacgccgctcgcagcaccggagagggcgcactgcaaaggcgggcagcagaccgtggagagcccgggagcggagctggacaccgcctcggagggaagaaatgaggtagcggcggttcccggacccggccatgcccgtcccctgttctcggagcccagcgccgtctcggccaggccagcccggacactgagcgggccgagcgcgagtccccggcgtccggcggagcgaagatgcagtgagtccccgcgggactgctgcgcggggcccgccgcggccagccggacccagcatccgaccgcactttgggcgagctgctgacttgagaccagcccaaacggggggctttccatctccagcacccctcggaggtggggagcaccggcccctaggcacactcgctgtagagtttccgcgggttttggccccagtcctgagggttgtgtgtgttaggggacccacctcacgttcgccgaggagttcctgcatattcttgcaccatcccgggtgctgttgctggggctctctttatttgcacgcgcgcttctagcttttttcctgacattttccactctacgctcgttctttctcccttgcattttgttgcttgcctgagactctttgctctcgcccttgcccaggctggggtaattctgtgtgcgcgcgtgtctcccccgcaccaaccttttttgcacactcgaagctgaatattttcttttttagagaatttacccgcacctcctgcggggttcctaagcactctctctgctcccctccccccaactccctaccacagggccgctcccagtagttttattcttcgatcttgcccgggccgagcctggcaggggccggtggctcagcgggcctcgcacccggcgctccgccgccgccgccgcccagctgcgccggggcgccctccggagatgctgccgtggaagaagcacaagttcgagctgctggccgaggcgccgccgcggcaggcgtccaagcccaagggctacgctgtgagcctgcactactcggcgctcagctcgctggcgcgggcgtgccccgaaggcgcgcttagccgggtgggcagcatgttccgctccaagcgcaagaagctgcacatcactagcgaggacccaacttacaccgtgctctacctgggcaatgccaccaccatccaggcgcgcggcgacggctgcaccgaccttgctgtgggcaagatctggagcaagagcgaggcgggccgtcagggcaccaagatgaagctgacggtgagtgcgcagggtatccgcatggtgcacgccgaggagcgcgcgctgcgccgcccgggccacctctacctgctgcaccgcgtcacctactgcgtggccgacgcgcggctgcccaaggtcttcgcctgggtgtaccggcacgagctgaagcacaaggccgtgatgctgcgctgccacgccgtgctggtgtccaagcccgaaaaggcgcaggccatggccctgctgctctaccagacgtcggccaacgcgctggcggaatttaaacgcctcaagcggcgggacgacgcgcgtcaccagcagcaggagctggtgggcgcacacaccatcccgctagtgccgctgcgcaagctgctcctacacggaccctgctgctataaaccgccggtggagcgcagccgcagcgcgcccaagcttggctccatcaccgaggacctgctcggcgaacagctggagcaggagctgcaggaggaagaggaagaggagcaacccgagggctgcccggaggaggaggagaaccgtgcggcagagggagatccagcagaggaggaggccgaggcgcagcgtgcgctagtggtcgccatgcactttgagtgcggggacttgttggatactctggagaatggccgtggggaggcgctaggaggcggcgggggctccctgggcccgggggccgggccgccgcctctgctgctgggcagcgcctccgacatgaaggctgagctgtcgcaacttattagcgacctgggcgagctcagcttcggcaacgacgtgcgcaccctgcaggccgacttgcgggtgacgcgcctgctgtcaggcgacagcacgggcagcgagagctccatcgagggcgggggccctgacgccacctccgccaccgccggggactcgtcccgccaggccgacggcgccagtgcagacgagccccactcgggctgagctcctccgcgcgtcgccggcgctccaccgtggctacccatccgtggtcccgacaacctccctgtcccttgcccgcccccaggaagggggaaatggggcatttggggcccagacctacacttggagcccaggtccagcgttcccccgaccgctttcccctacctcccggcccccgctcccgccccagcacttttggctctgttgcgcgtggggatgcggggagatttgagaggggaaaaccccgccaggagggagagagaggcacccctctgggatgcgggtgagggaaggttggctgaagttcctagtctcaggccgtaggtgcctggccagtttcctgtttgtggggcagctggggcctgaggaggaggggttcacttcctcctcccaccccctgggagcggccctgcgctgtcactgacatctcattaaaaaaaaaaaaaaatttgctctcaaggtgtttgaggctttaatgcaaccctttagcccttggttctttttggtgcaagaattctggctgtttacctcagactcagacccctgaaatgttgccaaattcttcaaataactgtttggggggtggggggagatgaaagagagtcgcgttttgtttacagttaaagacatccaatatcttaaaaaggagttttcctttagaaacacacacacccttcctcttgctcaaaagatctcactccatgatactgtgtaaaatatttttgcactgttgtgaagtatttttgacttttttctgtacataactgtgttctcagagctgaatgtttatatcttttgctgtgcaaaagaaacatgtaaaatgttgttcagttgtatatacagaaatgtgtataaaacattttgttattttttaaaagtagcactgttctggttctgtttgcacgccagtggggagagaataaagaggaaaatttaacagaaaaaaaaaaaaaaaaaaaaaaaaaaaaaaaaaaaaaaa

SEQ ID NO: 58 - protein, 423 aa, Homo sapiens
MLPWKKHKFELLAEAPPRQASKPKGYAVSLHYSALSSLARACPEGALSRVGSMFRSKRKKLHITSEDPTYTVLYLGNATTIQARGDGCTDLAVGKIWSKSEAGRQGTKMKLTVSAQGIRMVHAEERALRRPGHLYLLHRVTYCVADARLPKVFAWVYRHELKHKAVMLRCHAVLVSKPEKAQAMALLLYQTSANALAEFKRLKRRDDARHQQQELVGAHTIPLVPLRKLLLHGPCCYKPPVERSRSAPKLGSITEDLLGEQLEQELQEEEEEEQPEGCPEEEENRAAEGDPAEEEAEAQRALVVAMHFECGDLLDTLENGRGEALGGGGGSLGPGAGPPPLLLGSASDMKAELSQLISDLGELSFGNDVRTLQADLRVTRLLSGDSTGSESSIEGGGPDATSATAGDSSRQADGASADEPHSG

SEQ ID NO: 59 - DNA, 2529 bp, Homo sapiens
  CDS (203)..(1996)
ggaggcgagcgaacagagggagggacccgcccgccgcgccccggccgctgggcatgtgtgtccgcaggcgcccgacgctgccgatgtcccggggctgagccgcgcccaggtgtcccggacagtgcgtgcgagcgtgtgtgtccgcgcaggcgagcaccgcgccggccctgagcctcccgctcgctccccacggccgcggtgcatgttcgcctcctgccactgtgtgccgagaggcaggaggaccatgaaaatgatccactttcggagctccagcgtcaaatcgctcagccaggagatgagatgcaccatccggctgctggacgactcggagatctcctgccacatccagagggaaaccaaagggcagtttctcattgaccacatctgcaactactacagcctgctggagaaggactactttggcattcgctatgtggacccagagaagcaaaggcactggcttgaacctaacaagtccatcttcaagcaaatgaaaactcatccaccatacaccatgtgctttagagtgaaattctacccacatgaacccttgaagattaaagaagagctcacaagataccttttataccttcagattaaaagggacatttttcatggccgcctgctgtgctccttttctgatgctgcctacctgggtgcctgtattgttcaagctgagcttggtgattacgatcctgatgagcatcctgagaattacatcagtgagtttgagattttccccaagcagtcacagaagctggaaagaaaaatagtggaaattcataaaaatgaactcagggggcagagcccaccagttgctgaatttaacttgctcctgaaagctcacactttggaaacctacggggtggatcctcacccatgcaaggattcaacaggcacaacaacatttttaggattcacagctgcaggctttgtggtctttcagggaaataagagaatccatttgataaaatggccagatgtctgcaaattgaagtttgaagggaagacattttatgtgattggcacccagaaggagaaaaaagccatgttggcattccatacttcaacaccagctgcctgcaaacatctttggaagtgtggagtggaaaaccaggccttttataagtatgcaaaatccagtcagatcaagactgtatcaagcagcaagatattttttaaaggaagtagatttcgatatagtgggaaagttgccaaagaggtggtggaggccagttccaagatccagagggagcctcctgaggtgcacagagccaacattactcagagccgcagttcccactccttgaacaaacagctcatcattaacatggaacccctgcagcccctgcttccttcccccagcgagcaagaagaagaacttcctctgggtgagggtgttccattgcctaaagaggagaacatttctgctcccttgatctccagctccccagtgaaggcagcccgggagtatgaagatccccctagtgaagaggaagataaaataaaagaagaacctttaaccatctctgaactagtgtacaacccaagtgccagcctgctccccacccctgtggatgacgatgagattgacatgctctttgactgtccttctaggcttgagttggaaagagaagacacagattcatttgaggatctggaagcagatgaaaacgcctttttgattgctgaagaagaggagctgaaggaggctcgccgtgctttgtcgtggagctatgacattctgactggccatattcgggtgaacccactggtcaagagtttttccaggctccttgtggtgggcctgggactgctgctctttgtatttcccctgctcctcctccttttggagtcaggtattgatctctccttcttatgcgaaatccgccagacaccagagtttgagcagtttcactatgaatactactgtcccctcaaggagtgggtggctgggaaagtccacctcatcctctacatgctgggttgctcatgaagttaatctctcacgtgactaagggctatattcaatgctagtgatttctttttttcagcaaatgcctggttctgaagggtcacggggctgtcaacaggtgttccttactcataattgattattcaaacctttaagttagctttccataattcactgcacttaaataagtttaaatcaaatacagttattttagttacaggttaggaagatggtctttaaataaccaaaaatatgtttattttttattatagtgtagacatacccttcatctattatatcataatacatgttacattggactgaattagattttcccatttctaatagttggcaccattataagctataaggttcagaatcagaattttagtaacaactcaagagaaagttgttgaatataatccttagtgaaaacagtgtcctctaaccaatgcctatacaactaaatttatgctgggtttttggttctgtttttttaaaaatatttttatgtgttcaaactattttggtaaatttttagcaaaaaaaaaaaa

SEQ ID NO: 60 - protein, 597 aa, Homo sapiens
MFASCHCVPRGRRTMKMIHFRSSSVKSLSQEMRCTIRLLDDSEISCHIQRETKGQFLIDHICNYYSLLEKDYFGIRYVDPEKQRHWLEPNKSIFKQMKTHPPYTMCFRVKFYPHEPLKIKEELTRYLLYLQIKRDIFHGRLLCSFSDAAYLGACIVQAELGDYDPDEHPENYISEFEIFPKQSQKLERKIVEIHKNELRGQSPPVAEFNLLLKAHTLETYGVDPHPCKDSTGTTTFLGFTAAGFVVFQGNKRIHLIKWPDVCKLKFEGKTFYVIGTQKEKKAMLAFHTSTPAACKHLWKCGVENQAFYKYAKSSQIKTVSSSKIFFKGSRFRYSGKVAKEVVEASSKIQREPPEVHRANITQSRSSHSLNKQLIINMEPLQPLLPSPSEQEEELPLGEGVPLPKEENISAPLISSSPVKAAREYEDPPSEEEDKIKEEPLTISELVYNPSASLLPTPVDDDEIDMLFDCPSRLELEREDTDSFEDLEADENAFLIAEEEELKEARRALSWSYDILTGHIRVNPLVKSFSRLLVVGLGLLLFVFPLLLLLLESGIDLSFLCEIRQTPEFEQFHYEYYCPLKEWVAGKVHLILYMLGCS

SEQ ID NO: 61 - DNA, 1777 bp, Homo sapiens
  CDS (38)..(1468)
agaagcccagtagacaaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctcttttgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttccttaaaaaaaaaaaaaaaaaaa

SEQ ID NO: 62 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 63 - DNA, 2305 bp, Homo sapiens
  CDS (451)..(1392)
gttactgtcccgcgaacccacatccctacaaagcaggaaagtatgcttgggagaggccaagtgagtggggaatcagcccaaagccaggcgtccagggtctccctcacctgaagctgactttttccccaccttggacagagggcgggagatgccatccccactgaacccagtgctttcaccagccatattagctcccactcaccccccgtcgtggaagcctcggccgtcacacctgcagggccggggcgtgcatggcctcagggatggcctgttcagctgctgggtgactcgggtccaggtgcctcaccacctgctgagctctgtgtgatttctggacacttctgctcgttgcctttgggctcagtgaagagtctggagtttatctggagtgaggtggccggttcttggtgggatctgagcaggacagcgtctggctccttccctcggctcatggccctcagaatctgcgtcacatacaccccagctctcccgataggtctctgcactcgctgttgcctctgcctggaacagtctccctcctggtgtcattgtctccgtggtgtgtccttcctgaccttccacctccaccagtctgtcccccttggggacagggactcgttgctcatgttcacccggcaggctggacacttcgtggagggctccaaagccggcagatcccggggccgcctctgtctctcccaggccctgcgtgttgcggtgagaggagcatttgtgtctctgtggtttgctgctggagctggtgaccgggagagaaacaagggagacaagggtgcccagacaggtgcggggctcagccaggaggcagaagacgtggacgtgtcccgggccaggagggtcacagatgcaccacaaggcactctgtgtggcactgggaacaggaattctgggagtcagtctgcaagggtggtgggcgttgctcacctgggagaagcctttagagtgggcgttgagcaggccattagctcgtgccctgaggaggtgcatgggcggcatgggctctccatggaaattatgtgggcgcgaatggatgtggctctgcgctcacctgggcgaggacttctggccggtgccggggcactctgcatgaccctggcagaatcgagctgccctgactatgaaaggggaagaagagcatgcctgaccctccaccggcaccccacccctcactgctccacctggggcctgcctctgcgggtggctgggtcctggctgactgttgtgactgttgaggccctgggggggtggcgcatgggagttaggaggactggccaggtggggcccactatgcacccacccccagtgtcaggtgcttctcctctcctcctccaccacctcctcctcctcctcctcatcatcatcctcacttgttgaggacgtcctgtgtgccaagtggtttatatgcccagcctcatttaatcctcagaatgactccatgaggtagctactaaaaccccccacttaacagatgaggaaactgaggcctagagaagctcaacaagttgcctaagttccaagtttcctctcccaactctcctaccctctcctcttccttctcctttctcccattcttccctgcctcttccctaactagacaattttttattgagtgtctcccaggtgcaggcatgagccaggtgctgggaaaatcatgataacccagctccttctggtcattttctcagctggttagaggctgggaggacacgcaagttcagctccagccgactggggcattggtggtagcccctggagacattgtgcaatggggctacgaggctgcatctggctccagggaagcgtgttgcaatccatgagtgatgtctgccatgcgtacaggcatggagagtgaggcgcctgtactgtctttctgtagaccctagactggtggggcctctgaaatgcatccagacactgtgctgggtgtgttgcatggccctcccaaccaattcagtatttttctccccattttccagggagaaatctaaggcgtcagaatgtaaggttcttattggaacccaggctccagggtccctggttttctgtgacatcatgctgcaggaccctgtttctctctttgctttggctgctggggagctcagaagggagctttaggcttgctctcagtcaccacattagctcaggggtttgggcatttttgtcgccgtctcctttgggctctgtctcctccctgctgtgcttcctgaggagcaggccggatgtaagttatcaactataaataaaaccaagacttccgttctggctctcaaaaaaaaaaaaaaaaaaa

SEQ ID NO: 64 - protein, 313 aa, Homo sapiens
MALRICVTYTPALPIGLCTRCCLCLEQSPSWCHCLRGVSFLTFHLHQSVPLGDRDSLLMFTRQAGHFVEGSKAGRSRGRLCLSQALRVAVRGAFVSLWFAAGAGDRERNKGDKGAQTGAGLSQEAEDVDVSRARRVTDAPQGTLCGTGNRNSGSQSARVVGVAHLGEAFRVGVEQAISSCPEEVHGRHGLSMEIMWARMDVALRSPGRGLLAGAGALCMTLAESSCPDYERGRRACLTLHRHPTPHCSTWGLPLRVAGSWLTVVTVEALGGWRMGVRRTGQVGPTMHPPPVSGASPLLLHHLLLLLLIIILTC

SEQ ID NO: 65 - DNA, 3210 bp, Homo sapiens
  CDS (1688)..(2959)
cctctttctcccccctctctccctcctctcctctctctctcctctcttctctgaacctctcttctctttctcttttcttacatattctactagttgttttccccttcttcttctcctttctctcctttttctccctcctccttgtctcttttactccattcctgcagaagagagagggctaaacttaaaaaaaaaaaaaaggaggaggaggaggaggaggcacccccttcgtattcttcttatcgttattttatacatatatgattttttttggagggagggtgttggttgccggctgaagagcacttatttaaaatactaaaaaaagaacatttttgggcgatctccagggtttttttaactagctctgtgtgttatagcagaagaagcagaagaaggagcaagaaagaggaaaagaagaggattatttattcgacctactttggatgtctctctcgcttttcctttttcctttttttggcaattattttcttctgatttttattttttctatttcgctgtgatttcgtcgccggcgtgaattatcccgtatttttctcccccttccgtcacctcccgaaagaagaaggcagcgagagcccggcgccaccggcacaacaaaaagagcaaagtgtgtgatcttcctcgccggctgcctcccgctctccagcgctgccttcctgaatggctggctgcgtccggccctggacctggccccccgacacccgcgcgccctgatcgccggcggcagcctcgcccagcgccctgctcggctcaccgcgctccccgcactcccgagcccggcgagggctcccgccgggacagcggcggcgccgcgggcggccccggcctccgctcgcgctccggctgcggccccgactcctgctcggactccggcccgggtcccggctcctccagcggcgctcgccgcagcagctccggcggcagctccagcggcgcctgcagccgcgacctcctcctcctccgccgccgccgccgcctccgccctcgccggcttcctctatgtcggctcagcccgcgcgctgcgcgtagcccgagcggccggcgggcgggcgcccggcgcgggtgagcgactgtgtgtgcgagtgtgtgtgtgcgcgggggtgcgggcgaggcggagggcgagtgtgtgcgcgcgccgtggcccatgcccgccgcccccggcgctgcgccccgcgccgctcccggctgccgcctgtgccatttctgattttgcaacttggggaagaagaaaaaagcgagagaagggagcttgctcgccggggggtggggaggggggaaggagagcgcggcccccccaggaacggagcgcggggggagcgggcgaggggagcaggggtgttggggggggagcctgagagcctgggggggctgcaaaaagagagaaagaaaacagcaggaaccacaacaaaacgccagcagggcgggcgggcgcgcagcagcagcggggcggccgaggcagtagcggcggcagcggcggcggcggcggaggcagcggccggtgtccggctcgggctcggctcctgcgaccccggggcgcccggcgggccccccgccccctccccctccccccttccccttccccttcccctcccagcgcgcccgcgcgccccgcggccctcggcgagcagctcggctccccccagcgctccccgggcccaaagatatggcaatggtagttagcagctggcgagatccgcaggacgacgtggccgggggcaaccccggcggccccaaccccgcagcgcaggcggcccgcggcggcggcggcggcgccggcgagcagcagcagcaggcgggctcgggcgcgccgcacacgccgcagaccccgggccagcccggagcgcccgccacccccggcacggcgggggacaagggccagggcccgcccggttcgggccagagccagcagcacatcgagtgcgtggtgtgcggggacaagtcgagcggcaagcactacggccaattcacctgcgagggctgcaaaagtttcttcaagaggagcgtccgcaggaacttaacttacacatgccgtgccaacaggaactgtcccatcgaccagcaccaccgcaaccagtgccaatactgccgcctcaagaagtgcctcaaagtgggcatgaggcgggaagcggttcagcgaggaagaatgcctccaacccagcccaatccaggccagtacgcactcaccaacggggaccccctcaacggccactgctacctgtccggctacatctcgctgctgctgcgcgccgagccctaccccacgtcgcgctacggcagccagtgcatgcagcccaacaacattatgggcatcgagaacatctgcgagctggccgcgcgcctgctcttcagcgccgtcgagtgggcccgcaacatccccttcttcccggatctgcagatcaccgaccaggtgtccctgctacgcctcacctggagcgagctgttcgtgctcaacgcggcccagtgctctatgccgctgcacgtggcgccgttgctggccgccgccggcctgcatgcctcgcccatgtctgccgaccgcgtcgtggccttcatggaccacatccgcatcttccaggagcaggtggagaagctcaaggcgctacacgtcgactcagccgagtacagctgcctcaaagccatcgtgctgttcacgtcagacgcctgtggcctgtcggatgcggcccacatcgagagcctgcaggagaagtcgcagtgcgcactggaggagtacgtgaggagccagtaccccaaccagcccagccgttttggcaaactgctgctgcgactgccctcgctgcgcaccgtgtcctcctccgtcatcgagcagctcttcttcgtccgtttggtaggtaaaacccccatcgaaactctcatccgcgatatgttactgtctgggagcagcttcaactggccttacatgtccatccagtgctcctagaccttgggcgcttcccacctgccccgtccccctagagactcagaggacccacctgggccaaggactccaaagccgcggggacaccgggaagtgcagcgggccaggcaggctgggtgggagggaggagggccgagacaggagcagcccacccagcagaaatacaatccgagctacaaagcatgggaaaaagagactcttttaggatcagatctgtgagcacgttggcgaggaaaaacaaaaaaaaaaaaaaa

SEQ ID NO: 66 - protein, 423 aa, Homo sapiens
MAMVVSSWRDPQDDVAGGNPGGPNPAAQAARGGGGGAGEQQQQAGSGAPHTPQTPGQPGAPATPGTAGDKGQGPPGSGQSQQHIECVVCGDKSSGKHYGQFTCEGCKSFFKRSVRRNLTYTCRANRNCPIDQHHRNQCQYCRLKKCLKVGMRREAVQRGRMPPTQPNPGQYALTNGDPLNGHCYLSGYISLLLRAEPYPTSRYGSQCMQPNNIMGIENICELAARLLFSAVEWARNIPFFPDLQITDQVSLLRLTWSELFVLNAAQCSMPLHVAPLLAAAGLHASPMSADRVVAFMDHIRIFQEQVEKLKALHVDSAEYSCLKAIVLFTSDACGLSDAAHIESLQEKSQCALEEYVRSQYPNQPSRFGKLLLRLPSLRTVSSSVIEQLFFVRLVGKTPIETLIRDMLLSGSSFNWPYMSIQCS

SEQ ID NO: 67 - DNA, 6305 bp, Homo sapiens
  CDS (107)..(4021)
tttcccgcatgaaaattacttaaacgttgcacacaacgtttcacaaaatcttttgtgaaagaagaaaaggaaattcagtgtgtgagtctcagcaggagttaagctaatgcagcttaaaataatgccgaaaaagaagcgcttatctgcgggcagagtgcccctgattctcttcctgtgccagatgattagtgcactggaagtacctcttgatccaaaacttcttgaagacttggtacagcctccaaccatcacccaacagtctccaaaagattacattattgaccctcgggagaatattgtaatccagtgtgaagccaaagggaaaccgcccccaagcttttcctggacccgtaatgggactcattttgacatcgataaagaccctctggtcaccatgaagcctggcacaggaacgctcataattaacatcatgagcgaagggaaagctgagacctatgaaggagtctatcagtgtacagcaaggaacgaacgcggagctgcagtttctaataacattgttgtccgcccatccagatcaccattgtggaccaaagaaaaacttgaaccaatcacacttcaaagtggtcagtctttagtacttccctgcagacccccaattggattaccaccacctataatattttggatggataattcctttcaaagacttccacaaagtgagagagtttctcaaggtttgaatggggacctttatttttccaatgtcctcccagaggacacccgcgaagactatatctgttatgctagatttaatcatactcaaaccatacagcagaagcaacctatttctgtgaaggtgatttcagtggatgaattgaatgacactatagctgctaatttgagtgacactgagttttatggtgctaaatcaagtagagagaggccaccaacatttttaactccagaaggcaatgcaagtaacaaagaggaattaagaggaaatgtgctttcactggagtgcattgcagaaggactgcctaccccaattatttactgggcaaaggaagatggaatgctacccaaaaacaggacagtttataagaactttgagaaaaccttgcagatcattcatgtttcagaagcagactctggaaattaccaatgtatagcaaaaaacgcattaggagccatccaccataccatttctgttagagttaaagcggctccatactggatcacagcccctcaaaatcttgtgctgtccccaggagaggatgggaccttgatctgcagagctaatggcaaccccaaacccagaattagctggttaacaaatggagtcccaatagaaattgcccctgatgaccccagcagaaaaatagatggcgataccattattttttcaaatgttcaagaaagatcaagtgcagtctatcagtgcaatgcctctaatgaatatggatatttactggcaaacgcatttgtaaatgtgctggctgagccaccacgaatcctcacacctgcaaacacactctaccaggtcattgcaaacaggcctgctttactagactgtgccttctttgggtctcctctcccaaccatcgagtggtttaaaggagctaaaggaagtgctcttcatgaagatatttatgttttacatgaaaatggaactttggaaattcctgtggcccaaaaggacagtacaggaacttatacgtgtgttgcaaggaataaattagggatggcgaagaatgaagttcacttagaaatcaaagatcctacatggatcgttaaacagcccgaatatgcagttgtgcaaagagggagcatggtgtcctttgaatgcaaagtgaaacatgatcacaccttatccctcactgtcctgtggctgaaggacaacagggaactgcccagtgatgaaaggttcactgttgacaaggatcatctagtggtagctgatgtcagtgacgatgacagcgggacctacacgtgtgtggccaacaccactctggacagcgtctccgccagcgctgtgcttagcgttgttgctcctactccaactccagctcccgtttacgatgtcccaaatcctccctttgacttagaactgacagatcaacttgacaaaagtgttcagctgtcatggaccccaggcgatgacaacaatagccccattacaaaattcatcatcgaatatgaagatgcaatgcacaagccagggctgtggcaccaccaaactgaagtttctggaacacagaccacagcccagctgaagctgtctccttacgtgaactactccttccgcgtgatggcagtgaacagcattgggaagagcttgcccagcgaggcgtctgagcagtatttgacgaaagcctcagaaccagataaaaaccccacagctgtggaaggactgggatcagagcctgataatttggtgattacgtggaagcccttgaatggtttcgaatctaatgggccaggccttcagtacaaagttagctggcgccagaaagatggtgatgatgaatggacatctgtggttgtggcaaatgtatccaaatatattgtctcaggcacgccaacctttgttccatacctgatcaaagttcaggccctgaatgacatggggtttgcccccgagccagctgtagtcatgggacattctggagaagacctcccaatggtggctcctgggaacgtgcgtgtgaatgtggtgaacagtaccttagccgaggtgcactgggacccagtacctctgaaaagcatccgaggacacctacaaggctatcggatttactattggaagacccagagttcatctaaaagaaacagacgtcacattgagaaaaagatcctcaccttccaaggcagcaagactcatggcatgttgccggggctagagccctttagccactacacactgaatgtccgagtggtcaatgggaaaggggagggcccagccagccctgacagagtctttaatactccagaaggagtccccagtgctccctcgtctttgaagattgtgaatccaacactggactctctcactttggaatgggatccaccgagccacccgaatggcattttgacagagtacaccttaaagtatcagccaattaacagcacacatgaattaggccctctggtagatttgaaaattcctgccaacaagacacggtggactttaaaaaatttaaatttcagcactcgatataagttttatttctatgcacaaacatcagcaggatcaggaagtcaaattacagaggaagcagtaacaactgtggatgaagctggtattcttccacctgatgtaggtgcaggcaaagttcaagcagtaaatcccaggatcagcaatcttactgctgcagctgctgagacctatgccaatatcagttgggaatatgagggaccagagcatgtgaacttttatgttgaatatggtgtagcaggcagcaaagaagaatggagaaaagaaattgtaaatggttctcggagcttctttgggttaaagggtctaatgccaggaacagcatacaaagttcgagttggtgctgtgggggactctggttttgtgagttcagaggatgtgtttgagacaggcccagcgatggcaagccggcaggtggatattgcaactcagggctggttcattggtctgatgtgtgctgttgctctccttatcttaattttgctgattgtttgcttcatcagaagaaacaagggtggtaaatatccagttaaagaaaaggaagatgcccatgctgaccctgaaatccagcctatgaaggaagatgatgggacatttggagaatacagtgatgcagaagaccacaagcctttgaaaaaaggaagtcgaactccttcagacaggactgtgaaaaaagaagatagtgacgacagcctagttgactatggagaaggggttaatggccagttcaatgaggatggctcctttattggacaatacagtggtaagaaagagaaagagccggctgaaggaaacgaaagctcagaggcaccttctcctgtcaacgccatgaattcctttgtttaatttttaagctctttgccaatattccatttctctagaatgtttatcctaagcacttgtttgtcagccctctcatactatgaacatatgggtagagagtatattttctgctgtatgttagtattatgagaatagttacagcaaaaacataactcagtcaaatgatatgttaatatgaactggaatgcaaagtgcatactttttcattcaaaatgggtattcttgatttcctcagaactgataaaaaataatgcaacatcaccaacagatcctgttatttcctctgcaggatacagttcaatatgatgcatgaaaaatgctccacatttaaaggacatacccgtgtatgttatgaaaacatggtttgatactttgtttatactaccctcagctgaacccctatatatgaattccgttttcattgtcaagaatgttactgtagtattctctagaacttcaatgtctttgtggacattgttgtgaaattggtgactatgtatagctgtcgttagtctttttgggagactgttaggaacagtttgtacagtatatacttgctaaatgagttcattatgacagtcacattgctgatgcttactgagaactattacctactcttggctcctgttactccgtaggcttcttaatcttccaggcattacagcagcacagtgttctactttttacatcatttctatgttcggttgtttttaggcataaacaatgtgtattgcagtgcatttcggcatttgtgccatactgaaagaatcaaaaacaaatcatccaaattaaatttcaaacattatttcagagaacacagggcaagacacatacagtgccttcagatattaagcattccacaacatcgtgcattctgtatcagctggtccagtccattctgggtcctagattactgtcattgtctaaaagtaacttttaaaaagcagagttcatgaaaactgcaatgctgggaaaagaaggaaacatgaaaataaaaataagacagtttattagaaatagcatttcctcataagcataaaaagaaaatctttgttgccaactgaagcacatgatgattttgtggtcctttatggtttctattacattcagtaagaaagatgtcaacatgctagaaaattaattttaaaactaagttattccaacactaaaagcatacaacagcatgccaacagtaatatattattctccaagactttacctatgtaagtgttcaaaactctgcagcattaaacaacgtgtatgcaaattgttatggatacatttcagaatctaagaaatcaggcaagtgcttaaaaggccaacggtccaagggattacatctgcagtttaaaaagtaaatatatattctatcgtattcataaacaatatctatcaaatgggttacctccaaatatgaaaatctataacaacctatggttgaaggaatgctcagtttcatttgccaataaattggtttctcataacttgcatcaagtttaattttaagtaaagctttttatatgtagatattttgttgaatttgtaaatacacttaaaatgtagatgctatatgcttataggtgttacatacaaataaacatgcaatgtttatgttgtactgtataagaggtaagctaattaatgcagtgaatgggattggaaagcatctacttaaatatctattgggttcccccctccccccaccttttttgctgtgaaactgaaatagtgaacttttctacgtattgacagcagatttttcgatgaaatcttcagagctttgcctatggggcacagtaggcctagtaacctggcatgtttgatatatgtaggtaaagcataatttaaagtaatcccaggtaaagatggccctaaatactttcatgtctctatattcatttttcacagatccacctgtctcttgaaaatataaaaagacaaaacaggtttgccttggcatcagagagcacaaagattaaaagttactttaaatttgccaatattttgggagaacaataaaactacattttttcctcttccatactggtagatgcgaaatttatctgtgcatgaaagggtcacttctgtaatagtgcaacagatttggtattaaaaattaaatgtggttttaaaagttcctctctcttttgtaatttatgttcccaattgagtgtgaatgtccaagtaatggtgtatgtaatggtacaggcaaatgtgactggatttccctcaaaaaagtaactattaaacagtcttgatctctttgtgacttttaactttttcaaac

SEQ ID NO: 68 - protein, 1304 aa, Homo sapiens
MQLKIMPKKKRLSAGRVPLILFLCQMISALEVPLDPKLLEDLVQPPTITQQSPKDYIIDPRENIVIQCEAKGKPPPSFSWTRNGTHFDIDKDPLVTMKPGTGTLIINIMSEGKAETYEGVYQCTARNERGAAVSNNIVVRPSRSPLWTKEKLEPITLQSGQSLVLPCRPPIGLPPPIIFWMDNSFQRLPQSERVSQGLNGDLYFSNVLPEDTREDYICYARFNHTQTIQQKQPISVKVISVDELNDTIAANLSDTEFYGAKSSRERPPTFLTPEGNASNKEELRGNVLSLECIAEGLPTPIIYWAKEDGMLPKNRTVYKNFEKTLQIIHVSEADSGNYQCIAKNALGAIHHTISVRVKAAPYWITAPQNLVLSPGEDGTLICRANGNPKPRISWLTNGVPIEIAPDDPSRKIDGDTIIFSNVQERSSAVYQCNASNEYGYLLANAFVNVLAEPPRILTPANTLYQVIANRPALLDCAFFGSPLPTIEWFKGAKGSALHEDIYVLHENGTLEIPVAQKDSTGTYTCVARNKLGMAKNEVHLEIKDPTWIVKQPEYAVVQRGSMVSFECKVKHDHTLSLTVLWLKDNRELPSDERFTVDKDHLVVADVSDDDSGTYTCVANTTLDSVSASAVLSVVAPTPTPAPVYDVPNPPFDLELTDQLDKSVQLSWTPGDDNNSPITKFIIEYEDAMHKPGLWHHQTEVSGTQTTAQLKLSPYVNYSFRVMAVNSIGKSLPSEASEQYLTKASEPDKNPTAVEGLGSEPDNLVITWKPLNGFESNGPGLQYKVSWRQKDGDDEWTSVVVANVSKYIVSGTPTFVPYLIKVQALNDMGFAPEPAVVMGHSGEDLPMVAPGNVRVNVVNSTLAEVHWDPVPLKSIRGHLQGYRIYYWKTQSSSKRNRRHIEKKILTFQGSKTHGMLPGLEPFSHYTLNVRVVNGKGEGPASPDRVFNTPEGVPSAPSSLKIVNPTLDSLTLEWDPPSHPNGILTEYTLKYQPINSTHELGPLVDLKIPANKTRWTLKNLNFSTRYKFYFYAQTSAGSGSQITEEAVTTVDEAGILPPDVGAGKVQAVNPRISNLTAAAAETYANISWEYEGPEHVNFYVEYGVAGSKEEWRKEIVNGSRSFFGLKGLMPGTAYKVRVGAVGDSGFVSSEDVFETGPAMASRQVDIATQGWFIGLMCAVALLILILLIVCFIRRNKGGKYPVKEKEDAHADPEIQPMKEDDGTFGEYSDAEDHKPLKKGSRTPSDRTVKKEDSDDSLVDYGEGVNGQFNEDGSFIGQYSGKKEKEPAEGNESSEAPSPVNAMNSFV

SEQ ID NO: 69 - DNA, 5166 bp, Homo sapiens
  CDS (240)..(2501)
atttccattctggctgggaagggctggggctccactcagcctggagaccgaagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccagaaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagttcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaatactaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaatatgtaaaatatgcttataaatcagaaaaataaattgtttctcactaagtcaaaagaaaaaaagctcatgtttccaccac

SEQ ID NO: 70 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 71 - DNA, 11457 bp, Homo sapiens
  CDS (759)..(6443)
cgccgccgccgccgccaccgcgggagccagagctgccgggaggagcggcatccgcgccagactggagcgggagggcggcggagggcagttgctgggaatttttcagccgagagggcgagcgatccggagagagaccccgagagcttgggagcggtagggcgtgcgagcgccgcagccagcggagcaaacctcgaaatagatctggaaagccaggctcccggaggaaatgggactgtgaacgaaccggagagcaagaagggaaggaagcgccgggattgctgatgtcagaggagcccggaaagtcgcgctggaaaaatctgaagacagccggggctctgcttcttcctcaggagagacaccgccggccgcccccacacgccccctcggcgcctccgggtgccccctgagagccggcgacagcgcccagccgggctgctgcggggcgacggaggactgaggggcgcgcggagcggagaccgaggagcgacttcaggaatacagataagtgtctggtggcttgacgtggattttaatgaatttggactccatgtggatttggtcgtctccctgattccgagctgcgggcagggagaggggcctcgcgccgccctcagcagccggcggcggccgaggtagaccgagcggggacggaaggacagaccgacgtcgccgagctggaatcatgtgagggccaaccggggaaggtggagcagatgagcacacacaggagccgtctcctcaccgccgcccctctcagcatggaacagaggcggccctggccccgggccctggaggtggacagccgctctgtggtcctgctctcagtggtctgggtgctgctggcccccccagcagccggcatgcctcagttcagcaccttccactctgagaatcgtgactggaccttcaaccacttgaccgtccaccaagggacgggggccgtctatgtgggggccatcaaccgggtctataagctgacaggcaacctgaccatccaggtggctcataagacagggccagaagaggacaacaagtcttgttacccgcccctcatcgtgcagccctgcagcgaagtgctcaccctcaccaacaatgtcaacaagctgctcatcattgactactctgagaaccgcctgctggcctgtgggagcctctaccagggggtctgcaagctgctgcggctggatgacctcttcatcctggtggagccatcccacaagaaggagcactacctgtccagtgtcaacaagacgggcaccatgtacggggtgattgtgcgctctgagggtgaggatggcaagctcttcatcggcacggctgtggatgggaagcaggattacttcccgaccctgtccagccggaagctgccccgagaccctgagtcctcagccatgctcgactatgagctacacagcgattttgtctcctctctcatcaagatcccttcagacaccctggccctggtctcccactttgacatcttctacatctacggctttgctagtgggggctttgtctactttctcactgtccagcccgagacccctgagggtgtggccatcaactccgctggagacctcttctacacctcacgcatcgtgcggctctgcaaggatgaccccaagttccactcatacgtgtccctgcccttcggctgcacccgggccggggtggaataccgcctcctgcaggctgcttacctggccaagcctggggactcactggcccaggccttcaatatcaccagccaggacgatgtactctttgccatcttctccaaagggcagaagcagtatcaccacccgcccgatgactctgccctgtgtgccttccctatccgggccatcaacttgcagatcaaggagcgcctgcagtcctgctaccagggcgagggcaacctggagctcaactggctgctggggaaggacgtccagtgcaccaaggcgcctgtccccatcgatgataacttctgtggactggacatcaaccagcccctgggaggctcaactccagtggagggcctgaccctgtacaccaccagcagggaccgcatgacctctgtggcctcctacgtttacaacggctacagcgtggtttttgtggggactaagagtggcaagctgaaaaagattcgggccgacggtcccccccatggtggggtccagtacgagatggtctctgtgctcaaggacggaagccccatcctccgggacatggccttctccattgatcagcgctacctgtacgtcatgtctgagagacaggtcaccagggtccccgtggagtcatgtgagcagtatacgacttgtggggagtgcctgagctctggggaccctcactgtggctggtgtgccctgcacaacatgtgctcccgcagggacaaatgccaacaggcctgggaacctaatcgatttgctgccagcatcagccagtgtgtgagccttgcagtgcatcccagcagcatctcagtatctgagcacagccggttgcttagcctggtagtgagtgatgctcctgatctatctgcgggtatcgcctgtgcctttgggaacctgacagaggtggaggggcaggtgtccgggagccaggtcatctgcatctcacctgggcccaaggatgtccctgtcatcccgctggatcaagactggtttgggctggagctacagctgaggtccaaggagacagggaagatatttgtcagcaccgagttcaagttttacaactgcagtgcccaccaactgtgcctgtcctgtgtcaacagcgccttccgctgccattggtgcaagtaccgcaacctctgcactcatgaccccaccacctgctccttccaggagggccggatcaatatttcagaggactgtccccagctggtgcccacagaggagatcttgattccagtcggggaggtaaagccaatcacccttaaggcgcgaaatctgccccagccgcagtccggccagcgaggctatgagtgtgtcctcaacatacaaggagccatccaccgggtccccgctctgcgcttcaacagctccagcgttcagtgtcagaacagctcgtaccagtatgatggcatggacatcagcaatctggccgtggatttcgctgtggtgtggaacggcaatttcatcattgacaaccctcaggacctgaaagtccatctctacaagtgtgcagcccagcgggagagctgcggcctctgcctcaaggccgaccggaagtttgagtgtggctggtgcagcggcgagcgcaggtgcaccctccaccagcactgtaccagcccttccagcccctggctcgactggtccagccacaatgtcaagtgctccaaccctcaaatcaccgagattttgacggtgtctggaccgccggaaggagggacgcgagtgaccatccatggcgtgaacctgggtctggacttctccgagatcgcccaccatgtgcaggtggctggggtgccctgcacgcccctcccaggggaatacatcatcgctgagcagattgtctgtgagatgggccatgccctcgtgggaaccacctccgggccagtacgcctgtgtattggcgagtgtaagccagagttcatgacgaagtcccatcagcagtacaccttcgtgaacccttctgtgctgtcactcaacccaatccgaggtcccgagtcaggaggcactatggtgaccattaccggccattaccttggggctgggagcagcgtggcagtctacctgggcaaccagacctgcgagttctacgggaggtcaatgagtgagatcgtgtgtgtctcacccccatcatccaatggccttggcccggtccctgtttctgtgagtgtcgaccgagcccatgtggatagcaacctgcagtttgagtacatagatgaccctcgggtccagcgcatcgagccagagtggagcattgccagtggccacacacccctgaccatcacaggcttcaacctggatgtcattcaggagccaaggatccgagtcaaattcaatggcaaagaatctgtcaatgtgtgtaaagttgtgaacacaaccaccctcacctgcctggcaccctctctgaccacggactaccgccctggcctggacactgtggaacgcccagatgagtttggatttgtctttaacaatgtccaatccttgctaatttacaacgacaccaagtttatctactaccccaacccgacctttgaactgcttagccctactggagtcttggatcaaaagccaggatcgcccatcattctgaagggcaaaaacctctgccctcctgcctctggaggggccaaactcaactacactgtgctcatcggagagaccccttgtgctgtcaccgtatctgagacccagcttctctgcgagcctcccaacctcaccgggcagcacaaggtcatggttcacgtgggcgggatggtgttctcgcctggctcggtgagtgtcatctcagacagcttgctgaccctgccagccatcgtcagcatcgcggccggcggcagcctcctcctcatcatcgtcatcatcgtcctcattgcctacaagcgcaagtctcgagaaaatgacctcactctcaagcggctgcaaatgcagatggacaatctggagtcccgtgtggccttggagtgcaaggaagcttttgctgagctccagacggatatcaatgagttgaccagtgacctggaccgctcaggaatcccttacctggactatcgtacctacgctatgcgagtcctgttcccgggcatcgaggaccaccccgtcctgcgggagctggaggtacaaggaaacgggcagcagcacgtggagaaggccctgaagctctttgcccagctcatcaacaacaaggtgttcctgctgaccttcatccgcaccctggagctgcagcgcagtttctccatgcgcgaccggggcaacgtggcttcgctcatcatgaccggcctgcagggccgcctggaatatgccactgatgtcctcaagcagctgctctctgacctcatcgataagaacctggagaacaagaaccaccccaagctgctactccggaggacagagtctgtggctgaaaagatgctgaccaattggttcgccttcctcctgcacaagttcctaaaggagtgcgcaggggagccactcttcatgctatactgtgccatcaagcagcagatggagaagggccccattgatgccatcacgggcgaggcccgctactccctgagcgaggacaagctcatccggcagcagatcgagtacaagaccctgatcctgaactgcgtcaaccctgacaacgagaacagtccagagatcccagtgaaggtgttaaactgtgacaccatcacacaggtcaaggagaagattcttgatgccgtgtataagaatgtgccctattcccagcggccgagggcagtggacatggacttggagtggcgccaaggccggatcgcccgggtcgtgctgcaagatgaggacatcaccaccaagattgagggtgactggaagcggctcaacacactgatgcattatcaggtgtcagacaggtcggtggtggctctggtccccaaacagacctcctcctacaacatccctgcctctgccagcatctcccggacgtccatcagcagatacgactcctccttcaggtatacgggcagccccgacagcctgcggtcccgggccccgatgatcaccccagacctggaaagtggggtcaaggtgtggcatctggtgaagaaccatgaccacggtgaccagaaggagggtgaccggggcagcaagatggtgtccgagatctacctgacccggctactggccaccaagggcaccctgcagaagtttgtggacgacttgtttgagaccttgttcagcactgtgcaccggggcagcgctctccccctggccatcaagtacatgtttgatttcctagatgagcaggcagacaggcacagcatccatgacacagatgtgcggcacacctggaaaagcaactgcctccctctgcgcttctgggtgaacgtgattaagaacccccagttcgtgtttgacatccacaagggcagcatcacggacgcctgcctctctgtggtggcccagaccttcatggactcttgttcaacgtcagagcaccggctgggcaaggactccccctccaacaagctgctctatgccaaggacatccccagctacaagagctgggtggagagatactacgcagacatcgccaagctcccagccatcagtgaccaggacatgaatgcctacctcgccgagcagtcccgcctgcacgccgtggagttcaacatgctgagtgccctcaatgagatctactcctatgtcagcaagtatagtgaggagctcatcggggccctagagcaggatgagcaggcacggcggcagcggctggcttataaggtggagcagctcattaatgccatgtccattgagagctgagaggaggagcctcgcattcctgggaagagggacctgtccaagctgtcacactgggagtctcagatggaaggacaagtgatggggatcaggccccagagcttgctgtcccctgagaccccatcctggggagaggggaggactcctctccctacgccagccaagtttcgtcatagccagttccagctgggagagacagtgggcgtcgtccatcctcagtgagaacaccagagaacccggggccgggagaaggtggttcttcaagccgagaggcacgagctggggacagttctgcctctgtgactgctgctttgcatgaaaactcatttgatgtatattggggaaataatgagaactttatttaatttttttaagaaaaagggaaaaaaacagaaataaaacaaaaagccgccctgttaatcccgtccaacttttgtttaattctgatttctgtctcccttccatcttttctcccattcctccttctttatataatgcctatttccaaatgccagagaaagcagagatgctgagagacattggagagaaaatgactgtctccttttccttgaaattaaaaaaaaaaaaaaaaagagaaagaggagaagaagaatgatgagcacaagtatgcaccaaacacttcgcaaaaacagaggccagtaaaacctggaattatcccggcagccagaggagtatggaacttccagaactttgcacaaattgcaaagccatcaagagctcaccctggctgactggaaactgagctttatctaccacacacctgtatattctcatcttttgagaggagatgtgtacctagatagtaccaatgctttttgctactgttttttgttttgttttatttaatcctaaacctcaacaaatgaggagctggtctttgatatgtttcctttcaatttccctaaagttactatgagaagtggggtgaggtgggcctctcccagaccagacacctggcagccctgcctcatatcaatccctgtcataaaccaggcaccctggggaaacggcctggaggtgtgtgggccaggcctccacgaggttccatttgaaagttgatttggagacataggtgtttgactttggagttcactccaatcatccagtggtccctggcaattaaaaagaaaacaaaaatcaaacactgtttacagcaagcaatacttgaagagcataggttacagaagctgcagtatttattattatgctttctttctttcattctctcgtgcctggagaggggagaccacccttcgctcatatacggaagctcctgaccatctgggcctacagcacttcctcagtagaaatgactgtggcatgcccacgttactaccttctgcctctctttctgcctctcacggacttgtgagtgtgaaggacaagtggatgacttctcactggactttccttctctgtctcttctgatgatgcctaaaactatggataaccaattctcctgagtgtagattccaaacaaagagaccaaagctccatgcccaggtccaaaggccccatagaagccagtgggagtcatcgagaggaaaggcgctctgtgagtgtcaggaccttggtggccaggatagccatcagagtgtccaggcctcccacattaccttggatccgagcagccagctccaactgttctgcaagcagcactggagtcagggggtaggaggacaagtggaagcaaaggtgtgggactggggaagacaaagaggaacaagctgcccttcctcacttttcaaagggtcaggaatcctaggctatgatgctgggaagctagaccagctcctccaagagagactagaccagggtcattttctctgttattaactctgggctccagcttctccgtgccctgctttacctccaagtggttccaatttccaaaggccctgctgaccacatgtgattcccaggagagggctgggggaggggagcgcagaggtctggcttccatccttggcgtgtagctttggatcgctgtctaaccacacagcagacgttgcccggtctccccagctctagtttcttgcctgaatgcggctgacaaatgggaagagaaaagcattcagcaaaatactcaggaaacttgctgttttcattataattcacaaccagccatgccaaggccactttcttttgaaaatccacttctttaaagtttctcaggccctattagtagcctgaaggaaatactaatgactggccttccgcactaagccaaagtgtttgctcttcatagcactcaaagcttatcagcgcagagcccataatttatggagataaaaggaaaggagatataggtaagaagagtgtgaccaggagaccttatgctacctgtaaaaaagttcagcccaccccatctaacttctgagctctgtttgggtgaagattttctggccgcatggctgctcagactggcatccaggctttgctccaccaagaagttaaaggcagtcggacatctcagtagcatctctaccagcccttaactcaatgcatctacctggcatctcccagcagttacttttggagacgattcactgcccctggggcgtttccttgaaggtttgtggagagcgtggagaatgatggggcaatggccaattggagggtggagagtgaagagccggagctggctgtgagtggtttggccacatttctcaggattccatgagagacttggggaacttgggctgacaaaggaagtagcctggggcatccttaaggaaggaattaagaaaagggaaaaagctggactcaagccacgccatgagggtgaaaggttataaggccctgccccctttccagctgcccacctgttcctcctccacacctcttcgctttgggccaccaagaaccaatgaagtccacaccctttggatgagaaaaagagggagttggttggcctctcttctccctgttatccaatttgaggatattttgaccttgggtaaggatgaagtgttaaagccacagctcctctccacaagaagccattcatcttgggggaggcagagagggaagtctctctccaaagtctatccagcttcgcttcgtttcattgatctgcacaagagacaatgctctggaaaaggaagaggaccccagaagggtgcttggcaagacagaggatgctaatgggcaatggagagcactccctccagctggcccctgctgctgcctcccgtcctctgcatggggtcaggtgcttctgtgcttgctgtcctacctctctccacagcagggctctcaaaaccattttgatcccccattggcagagggttcccctctttacagagttcagtcattaaaagcatggatcagctgttaatctcattggaggagggaactgtttcctgcattcattcatctgggaaccttcttgagtagccactgtctgccagccactgctctagagatgggaaaacagcacggaacaaaaccaaggtctttcttccagcgaatttatatccttcaggaagctggttcctgccaccaacttagcaggcaacagttctcctcccctagtggcacagggtaccagttttgtaggaaaagtggtccagcaaaggaagaaagcagaccaacccagctgccttaccttattctggggccattcccccagcgatgagagctgctcttgtttctactgccaccatctcttctggctgcacttcacctgctgcttgagcttctgaccttccttcagttccaccaaatgaggacaggaaatagcagtcaagacccctggccctgctgagcgtgaaacagaagcaatggatgagtgctggacgaagaatggcctgggcagaacaaatagggagcatttgaaagcttctggctgataaatctccaggtgcatcccggttgccacgcctgcccccattaacctgctcctggtaaatactgatccagcagctgctccaggagaggccgtctttttttcccagccacgctgtgtcttgcatgagactcctggggcctgggcacagagagaaaagaattgagactcaggaggctcagtgggtgagaaaatgcaaagtggcttcacagacacagggctgtgggagcagatcgacggggaacttgggagatgaacttcagggccttccgacgccttgtctcaggaacatgctttgagaaaaatggtagcatcctttccataactcagtctctcttccctagtttccctgaagtgtgacgttttagtatctggagctcagtgatccccatgaatgagggataaagtttcactcttggtattttctaactagtgctagggaaagtcctgagacacgatcacagccactgcttggcatacagggcctccacccaataagcaaactggagattcctcagcctctcgtggacacccacatctcattcttctcacagcagagaagctctcccttcagcctgagctgtcttctttctgctgcagtgcagcctgctccctcctaccctggcctcaaggaaggtaggaaacatcttctgcatttcaaagtcctcactttgacttatttggccttcatcttggcatggaaggtggcaggcagaatggaaatactccccccaaacagaacagatattcttgcgtgtgtaagggcagaagggacaagctctctatcccatgagactaggggccggagcccacctgcctttccccacaacttttcctgctcaaacccactcctcttgacacactggaatctgtattatatatatttttaagaaaatacaatgatggttgtctggttttgttgtttttacaggtgttgtggaataaaaactgtaagaaaattaagtatttaaaatgttccaataaagtggggttttttgttattctaatatattattgtgtacctattgtaaatatgaaacactcctattttgcaagctgaggacacaatttgtactgttgttatatataaataaagtttactgaattaaaaaaaccttaaatctttaaataaaaaaaaaaaaaaaa

SEQ ID NO: 72 - protein, 1894 aa, Homo sapiens
MEQRRPWPRALEVDSRSVVLLSVVWVLLAPPAAGMPQFSTFHSENRDWTFNHLTVHQGTGAVYVGAINRVYKLTGNLTIQVAHKTGPEEDNKSCYPPLIVQPCSEVLTLTNNVNKLLIIDYSENRLLACGSLYQGVCKLLRLDDLFILVEPSHKKEHYLSSVNKTGTMYGVIVRSEGEDGKLFIGTAVDGKQDYFPTLSSRKLPRDPESSAMLDYELHSDFVSSLIKIPSDTLALVSHFDIFYIYGFASGGFVYFLTVQPETPEGVAINSAGDLFYTSRIVRLCKDDPKFHSYVSLPFGCTRAGVEYRLLQAAYLAKPGDSLAQAFNITSQDDVLFAIFSKGQKQYHHPPDDSALCAFPIRAINLQIKERLQSCYQGEGNLELNWLLGKDVQCTKAPVPIDDNFCGLDINQPLGGSTPVEGLTLYTTSRDRMTSVASYVYNGYSVVFVGTKSGKLKKIRADGPPHGGVQYEMVSVLKDGSPILRDMAFSIDQRYLYVMSERQVTRVPVESCEQYTTCGECLSSGDPHCGWCALHNMCSRRDKCQQAWEPNRFAASISQCVSLAVHPSSISVSEHSRLLSLVVSDAPDLSAGIACAFGNLTEVEGQVSGSQVICISPGPKDVPVIPLDQDWFGLELQLRSKETGKIFVSTEFKFYNCSAHQLCLSCVNSAFRCHWCKYRNLCTHDPTTCSFQEGRINISEDCPQLVPTEEILIPVGEVKPITLKARNLPQPQSGQRGYECVLNIQGAIHRVPALRFNSSSVQCQNSSYQYDGMDISNLAVDFAVVWNGNFIIDNPQDLKVHLYKCAAQRESCGLCLKADRKFECGWCSGERRCTLHQHCTSPSSPWLDWSSHNVKCSNPQITEILTVSGPPEGGTRVTIHGVNLGLDFSEIAHHVQVAGVPCTPLPGEYIIAEQIVCEMGHALVGTTSGPVRLCIGECKPEFMTKSHQQYTFVNPSVLSLNPIRGPESGGTMVTITGHYLGAGSSVAVYLGNQTCEFYGRSMSEIVCVSPPSSNGLGPVPVSVSVDRAHVDSNLQFEYIDDPRVQRIEPEWSIASGHTPLTITGFNLDVIQEPRIRVKFNGKESVNVCKVVNTTTLTCLAPSLTTDYRPGLDTVERPDEFGFVFNNVQSLLIYNDTKFIYYPNPTFELLSPTGVLDQKPGSPIILKGKNLCPPASGGAKLNYTVLIGETPCAVTVSETQLLCEPPNLTGQHKVMVHVGGMVFSPGSVSVISDSLLTLPAIVSIAAGGSLLLIIVIIVLIAYKRKSRENDLTLKRLQMQMDNLESRVALECKEAFAELQTDINELTSDLDRSGIPYLDYRTYAMRVLFPGIEDHPVLRELEVQGNGQQHVEKALKLFAQLINNKVFLLTFIRTLELQRSFSMRDRGNVASLIMTGLQGRLEYATDVLKQLLSDLIDKNLENKNHPKLLLRRTESVAEKMLTNWFAFLLHKFLKECAGEPLFMLYCAIKQQMEKGPIDAITGEARYSLSEDKLIRQQIEYKTLILNCVNPDNENSPEIPVKVLNCDTITQVKEKILDAVYKNVPYSQRPRAVDMDLEWRQGRIARVVLQDEDITTKIEGDWKRLNTLMHYQVSDRSVVALVPKQTSSYNIPASASISRTSISRYDSSFRYTGSPDSLRSRAPMITPDLESGVKVWHLVKNHDHGDQKEGDRGSKMVSEIYLTRLLATKGTLQKFVDDLFETLFSTVHRGSALPLAIKYMFDFLDEQADRHSIHDTDVRHTWKSNCLPLRFWVNVIKNPQFVFDIHKGSITDACLSVVAQTFMDSCSTSEHRLGKDSPSNKLLYAKDIPSYKSWVERYYADIAKLPAISDQDMNAYLAEQSRLHAVEFNMLSALNEIYSYVSKYSEELIGALEQDEQARRQRLAYKVEQLINAMSIES

SEQ ID NO: 73 - DNA, 2108 bp, Homo sapiens
  CDS (187)..(1353)
ggctccggcggcagcgggatctgcagttcggactccgcgcgccacagtcgctgcagttcgctccactctggtggccccgccgccctgcggggatcccgggggtcggcgggctgctcggacttggcgcggggccggcccggcctctctcttcctcggtggggcctagacggtcggggcaccgggaacatggagccctctccagccgctgggggcttggagaccactcgcctggtgagcccccgggaccgcggtggcgccggaggcagcctgcgtttgaagagtctcttcacagagccctcagagcccctccctgaggagtccaaacctgtggagatgcccttccaccactgccacagggacccccttccgccgccgggccttacccctgagaggctgcatgcacggaggcagctatatgctgcctgtgccgtttgctttgtcttcatggctggggaggtggtcggcgggtatctggcacacagcctggccatcatgaccgatgcagcccacttgctggcggatgtgggcagcatgatgggcagcctcttctccctctggctctccacccgtccagccacccgcaccatgacctttggctggcaccgttcagagactctgggggctttggcctctgtggtctccctctggatggtcactggcatcctcctgtacctggccttcgtccgcctgctgcacagcgactaccacatcgaggggggtgccatgctgctgaccgccagcatcgcagtctgtgccaacctgttaatggcctttgtgctgcaccaggctgggcccccccacagccacgggtctaggggagcagagtatgcaccgctggaggaggggcctgaagagcccctgcccctggggaacaccagcgtccgggcggcatttgtgcacgtgctgggggacctcctgcagagctttggggtactggctgcctccatcctcatctacttcaagcctcaatacaaggcagccgaccccatcagcaccttcctcttctccatctgtgcccttggatccaccgctcccaccctccgagacgttcttcgaatcctcatggaaggtaccccccgcaatgtggggttcgaacctgtgcgggatacgctgttgtcggtgccaggagtccgggcaacccatgagctgcacctgtgggcccttacgctcacttaccatgttgcctctgcacacctggccatcgactccaccgctgaccctgaagccgtcctggctgaagcctcatcccggctctactcccggtttggattctccagctgcaccctgcaggtcgagcagtatcagccggagatggcccagtgcctgcgctgccaggaacccccccaagcctgagccatggccctgccctcaccccactgccaggccgaggctcagccccagactctcagcatctgctgccctgatcacagagacgggaccgagccaggtccataccccttcctctctccctcctaccacctgccagtttccccagcctcagccccagccccagccccagtgggcaagaccaaagtgtggcggggagtggggtgggagtcaggggaatagatgtgactagttcaggggcggggactcccaggcctcagtgtggcagggtgtgttgaaggcctgtggtgccatctccccatggttcatgtggagccacgaacatcctttccctgcagtccatttgtctgtgtggcaggctggctggctgggggcatctgcctgtctatgtgctgttggtgtgcctatgcctgggggaggtcagtaggggccccctccccacatggccctcgctctgtctatgcaggggccccaaagcccgcactttgtccgtgtgtcttagccctgtggttttgtctgtgtgtgtgtgtgtgtgtgtgtgtgtgtgtgtgtgtgtgttcttggtgctgtggcctgtgtgtctctgtgcctatgtggctgtgctatggtttctatgagtctgctccatccatgtgtctgtttgggggtctatctctccatccctctgttggtgctgtgcccttggctatccctgaaagagggaggactccgctgcagctccaccaataaacttgtgtctcactgcaaaaaaaaaaaaaaaa

SEQ ID NO: 74 - protein, 388 aa, Homo sapiens
MEPSPAAGGLETTRLVSPRDRGGAGGSLRLKSLFTEPSEPLPEESKPVEMPFHHCHRDPLPPPGLTPERLHARRQLYAACAVCFVFMAGEVVGGYLAHSLAIMTDAAHLLADVGSMMGSLFSLWLSTRPATRTMTFGWHRSETLGALASVVSLWMVTGILLYLAFVRLLHSDYHIEGGAMLLTASIAVCANLLMAFVLHQAGPPHSHGSRGAEYAPLEEGPEEPLPLGNTSVRAAFVHVLGDLLQSFGVLAASILIYFKPQYKAADPISTFLFSICALGSTAPTLRDVLRILMEGTPRNVGFEPVRDTLLSVPGVRATHELHLWALTLTYHVASAHLAIDSTADPEAVLAEASSRLYSRFGFSSCTLQVEQYQPEMAQCLRCQEPPQA

SEQ ID NO: 75 - DNA, 1722 bp, Homo sapiens
  CDS (85)..(879)
attcattgcgccgcggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 76 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 77 - DNA, 3888 bp, Homo sapiens
  CDS (59)..(3709)
aaaactcagggaagcccagggcccgtgttgtgcttttggcccaggtaggtggacagacatgtcccaggcgggggacgtagaaggccccagcacaggagaccctgtgctcagtccccaacacaactgtgagcttttacagaacatggaaggagccagctccatgccaggcctgtcaccagatgggccgggagcaagctctgggcccggagtcagggctggcagcagaaggaagatccccaggaaggaggcccttcgaggtggcagctcccgggctgcaggtgctgctgaggtccggccaggggtcttggagctgctagctgtggtacagagccggggctcgatgctggctcctgggctccacatgcagctgccctcggtgcctactcaggggagagctctgacctccaagaggctccaggtttctctgtgtgacatcttagatgacagttgccccaggaaactttgtagcaggtctgctggcctcccagagagagctctggcctgcagggagaggcttgcaggagtggaggaggtgagctgcctcaggcccagggaggccagagacggtggaatgagttctccagggtgtgacagaagaagccccacactcagcaaagaggagccccctggaaggcccctgacatcctcaccagacccagtccctgtgagggtaagaaagaaatggaggaggcaaggggctcattcagagtgtgaggaaggggctggtgacttcctgtggcttgatcagagccctcgtggggacaacctcctgtctgtgggagaccctccccaagttgctgacctggagtccttgggaggcccttgcagacctccctctccaaaagacactgggtctgggcctggagagccaggtggaagtggggcaggatgtgcctcagggactgagaaatttggatatttgcccgctacaggggatgggccccagccaggcagcccctgtggccctgtcgggttcccagtgcccagtggaggggagtccctcagttcagctgcacaggctcctccacagagcgcagcactgtgcctgggggcgtcagcacaggcctctgcagagcagcaagaagctgtgtgtgtcgtgcggactggcagcgatgaaggccaggctccagcacaggaccaggaggagctggaggccaaggctcagccagcttccaggggaaggctggagcaaggactcgctgcccccgctgacacctgtgccagctcccgggagcccttgggcggcctcagctcctccctggatactgaagccagcagggcctgctcaggcccattcatggagcagagaagatccaagggcactaagaacctgaagaaaggtccagtgccctgtgcccaagaccggggcacagacagaagctcagacaactcccaccaggacaggccagaggaacccagcccaggaggctgccccagactggaggaagtgaaaataccccatggagtgaagcttgtgtgctacctgggttccgggccagtgatccagctcctgggggccatcagccacggccaggcaggggggcagctgccaccaaagctggaggttctagaggacttgatggaggtcagctcaccctcacctgcccagaggctcagaaggaagaaaaggcccatggtgcagggccctgctgggtgccaggttttccagccttctccttcaggaggcacagcaggggaccctggtggcctctctgaccccttctaccctccaagaagcggttccctggcccttggcgaccccagctcggaccctgcatgttcccagagtggcccaatggaggctgaagaggattctcttccggagcagccagaggactcagctcagctccaacaggagaagccatccctgtatattggggtgcggggcactgttgtccgttccatgcaggaggtactatggactcgccttcgggagctcccagacccagtgctgagtgaggaggtggtggagggcattgctgctggcattgaggcagccctctgggacctgacacaaggcaccaatggccggtacaagaccaagtatcgcagcctgctgttcaacctgcgggaccccaggaacctggacttgtttctcaaagtggttcatggagatgtcaccccctacgacctggtgcggatgagctcgatgcagctggccccccaggagctggcccgctggcgggaccaggaggagaaaaggggcctgaatatcattgagcagcaacagaaggagccgtgcagacttccagcctccaaaatgacccacaagggcgaagtggagattcagcgggacatggaccagacactgaccctggaggatctggtgggaccgcagatgttcatggactgcagcccacaggccctgcccatcgcatcagaggacaccacggggcagcatgaccaccacttcttagaccccaactgccacatctgcaaggactgggagccctcgaatgagctgctaggctccttcgaagccgccaagagctgcggggacaatatcttccagaaagccctaagccaaactcctatgcctgctccagagatgcccaaaaccagggagttgtctcccacggaaccacaggacagggtccctccatctgggctccatgtgcctgctgcacccacaaaggccctgccctgcctgccaccctgggaaggtgttctggacatgttctccatcaagcggttccgggccagggcccagctggtctcgggacacagctgtcggcttgtccaggctctgcccaccgtgatccgctcggcaggctgcatcccctccaacattgtctgggaccttctggccagcatctgcccagccaaggccaaggacgtctgcgtggtcagactgtgcccacatggggcccgggacacccagaactgccgcctgctctactcatacctcaatgataggcagcgccacgggctggcctctgtggagcacatggggatggtcctgctgcccctgcctgccttccagcccctgcccaccaggctgcgccctttggggggcccaggcctttgggctcttcctgtctcccctctcctttccccaggtctggaggtcactcactcaagtctgttgctggctgtgctgctccccaaggaagggcttccagacacagcagggtccagcccctggttggggaaggttcaaaagatggtctccttcaacagtaaggtggagaagagatactatcagccagatgacaggaggccgaatgtgcccctgaagggcacccctcccccaggaggtgcctggcagcagagccagggcaggggcagtatagctccaaggggaatctctgcttggcagaggccccccagaggcagggggaggctctggccagagcctgaaaactggcagcatcctgggcgagggcagtggcccccagagccaggcttgcgccagtcccagcatccctattcagtagcaccagctggtcatggctttggccgtggccagcacttccacagggactcctgtccccaccaagccctgctccggcacctcgaatccctggcgaccatgagtcaccagctccaagccttactgtgcccccagaccaagagctccatcccccgccctctgcagcgtttgtctagcgcccttgcagctccagagccccctggcccagcccgtgactcctctttggggcctacagatgaagctggctctgagtgtcccttccctagaaaggcctgaccctccttacccaccagaacaggggttttgatgccctcactagtgttgaagcctgttccagagagaggtgggactgcaaggagaggatggtcagccctacccacctgccctgtttgagcttcctgtttgacaatgtttgctgttgattttttgttcaataaagaatttggtaaaattgg

SEQ ID NO: 78 - protein, 1216 aa, Homo sapiens
MSQAGDVEGPSTGDPVLSPQHNCELLQNMEGASSMPGLSPDGPGASSGPGVRAGSRRKIPRKEALRGGSSRAAGAAEVRPGVLELLAVVQSRGSMLAPGLHMQLPSVPTQGRALTSKRLQVSLCDILDDSCPRKLCSRSAGLPERALACRERLAGVEEVSCLRPREARDGGMSSPGCDRRSPTLSKEEPPGRPLTSSPDPVPVRVRKKWRRQGAHSECEEGAGDFLWLDQSPRGDNLLSVGDPPQVADLESLGGPCRPPSPKDTGSGPGEPGGSGAGCASGTEKFGYLPATGDGPQPGSPCGPVGFPVPSGGESLSSAAQAPPQSAALCLGASAQASAEQQEAVCVVRTGSDEGQAPAQDQEELEAKAQPASRGRLEQGLAAPADTCASSREPLGGLSSSLDTEASRACSGPFMEQRRSKGTKNLKKGPVPCAQDRGTDRSSDNSHQDRPEEPSPGGCPRLEEVKIPHGVKLVCYLGSGPVIQLLGAISHGQAGGQLPPKLEVLEDLMEVSSPSPAQRLRRKKRPMVQGPAGCQVFQPSPSGGTAGDPGGLSDPFYPPRSGSLALGDPSSDPACSQSGPMEAEEDSLPEQPEDSAQLQQEKPSLYIGVRGTVVRSMQEVLWTRLRELPDPVLSEEVVEGIAAGIEAALWDLTQGTNGRYKTKYRSLLFNLRDPRNLDLFLKVVHGDVTPYDLVRMSSMQLAPQELARWRDQEEKRGLNIIEQQQKEPCRLPASKMTHKGEVEIQRDMDQTLTLEDLVGPQMFMDCSPQALPIASEDTTGQHDHHFLDPNCHICKDWEPSNELLGSFEAAKSCGDNIFQKALSQTPMPAPEMPKTRELSPTEPQDRVPPSGLHVPAAPTKALPCLPPWEGVLDMFSIKRFRARAQLVSGHSCRLVQALPTVIRSAGCIPSNIVWDLLASICPAKAKDVCVVRLCPHGARDTQNCRLLYSYLNDRQRHGLASVEHMGMVLLPLPAFQPLPTRLRPLGGPGLWALPVSPLLSPGLEVTHSSLLLAVLLPKEGLPDTAGSSPWLGKVQKMVSFNSKVEKRYYQPDDRRPNVPLKGTPPPGGAWQQSQGRGSIAPRGISAWQRPPRGRGRLWPEPENWQHPGRGQWPPEPGLRQSQHPYSVAPAGHGFGRGQHFHRDSCPHQALLRHLESLATMSHQLQALLCPQTKSSIPRPLQRLSSALAAPEPPGPARDSSLGPTDEAGSECPFPRKA

SEQ ID NO: 79 - DNA, 1903 bp, Homo sapiens
  CDS (190)..(1401)
agcatgagtcagacagcctctggctttctggaagggcaaggactctatatatacagagggagcttcctagctgggatattggagcagcaagaggctgggaagccatcacttaccttgcactgagaaagaagacaaaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgttttatacactttc

SEQ ID NO: 80 - protein, 403 aa, Homo sapiens
MQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 81 - DNA, 1203 bp, Homo sapiens
  CDS (76)..(783)
agaaagccgcgcacctcctcccgccaggcgctttctcggacgccttgcccagcgggccgcccgaccccctgcaccatggaccccgctcgccccctggggctgtcgattctgctgcttttcctgacggaggctgcactgggcgatgctgctcaggagccaacaggaaataacgcggagatctgtctcctgcccctagactacggaccctgccgggccctacttctccgttactactacgacaggtacacgcagagctgccgccagttcctgtacgggggctgcgagggcaacgccaacaatttctacacctgggaggcttgcgacgatgcttgctggaggatagaaaaagttcccaaagtttgccggctgcaagtgagtgtggacgaccagtgtgaggggtccacagaaaagtatttctttaatctaagttccatgacatgtgaaaaattcttttccggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaagaataatatgacagcatgaggaaacaaatcattggtgatttattcaccagtttttattaatacaagtcactttttcaaaaatttggatttttttatatataactagctgctattcaaatgtgagtctaccatttttaatttatggttcaactgtttgtgagactgaattcttgcaatgcataagatataaaagcaaatatgactcactcatttcttggggtcgtattcctgatttcagaagaggatcataactgaaacaacataagacaatataatcatgtgcttttaacatatttgagaataaaaaggactagcaaataaaacacaaaaaaaaaaaaaaaaaaaaaaaaaaaaaaa

SEQ ID NO: 82 - protein, 235 aa, Homo sapiens
MDPARPLGLSILLLFLTEAALGDAAQEPTGNNAEICLLPLDYGPCRALLLRYYYDRYTQSCRQFLYGGCEGNANNFYTWEACDDACWRIEKVPKVCRLQVSVDDQCEGSTEKYFFNLSSMTCEKFFSGGCHRNRIENRFPDEATCMGFCAPKKIPSFCYSPKDEGLCSANVTRYYFNPRYRTCDAFTYTGCGGNDNNFVSREDCKRACAKALKKKKKMPKLRFASRIRKIRKKQF

SEQ ID NO: 83 - DNA, 1903 bp, Homo sapiens; identical to SEQ ID NO: 79
  CDS (190)..(1401)

SEQ ID NO: 84 - protein, 403 aa, Homo sapiens; identical to SEQ ID NO: 80

SEQ ID NO: 85 - DNA, 938 bp, Homo sapiens
  CDS (1)..(468)
atggtcagctcctgttgtggctccgtgtgctctgaccagggctgcagccaagacctctgtcaggagacctgctgccgccccagctgctgtcagaccacctgttgcaggaccacctgctaccgccccagctgttgtgtgtccagctgctgcaggccccagtgctgccagtctgtgtgctgccaacccacctgctgtcgccccacctgctgtgagacgacctgctgccaccctaggtgctgcatctccagctgctgccgccccagctgctgtatgtccagctgctgcaagccccagtgctgccagtctgtgtgctgccagcccacctgctgccgccccagctgctgccgcccctgctgctgcctgcgtccagtctgtggccgagtctcctgccacaccacttgctatcgcccaacctgtgtcatctccacctgtccccgccccttgtgctgtgcctcctcttgctgctgagcccactgccctggctcacgtcccccttcaccactggcccacagatgtagacccttctactgtgctgaccattaggatacatgaagtggggttgatgtcattcaataggatggaccttatgcttccaaagagcccaccaccatttcactgactctgtgagaacattctggttcattttaaactccctcctttgctttctttttcttctggtggtggcaccaaatgtgaattaatttgtaatacactagctaagaaattattccaatcttctgatttccttattttctttatcactttaaggtacagattctccttctcagtgaggtagatattatctgcaggaccagttttgtcactgatgttgcaccctcagatccagccacccaattgtattctgtgtttctcctagggtgaatttcttatgctttgttgtatctctgctttctaataaacttttctgcacttaagaa

SEQ ID NO: 86 - protein, 155 aa, Homo sapiens
MVSSCCGSVCSDQGCSQDLCQETCCRPSCCQTTCCRTTCYRPSCCVSSCCRPQCCQSVCCQPTCCRPTCCETTCCHPRCCISSCCRPSCCMSSCCKPQCCQSVCCQPTCCRPSCCRPCCCLRPVCGRVSCHTTCYRPTCVISTCPRPLCCASSCC

SEQ ID NO: 87 - DNA, 1143 bp, Homo sapiens
  CDS (40)..(597)
ctcttggaaacccaaatagatccttcaccctctgacaccatggtcaactcctgttgtggctccgtgtgctctgaccaaggctgtggccaagacctctgccaggagacctgctgctgccccagctgctgtcagaccacctgctgcaggaccacctgctaccgccccagctacagtgtgtcctgctgctgcagaccccagtgctgccagtctgtgtgctgccagcccacctgctgtcgccccagctgctgtgtgtccagctgctgcaagccccagtgctgccagtctgtgtgctgccagcccacctgctgccaccctagctgctgcatctccagctgctgccgccccagctgctgtgtgtccagctgctgcaagccccagtgctgccagtctgtgtgctgccagcccaactgctgccgccccagctgcagcatctccagctgctgccgcccctcttgctgtgaatccagctgctgccgcccctgctgctgcctgcgtccagtctgtggccgagtctcctgccacaccacttgctatcgcccagcctgtgtcatctccacctgcccccgccccgtgtgctgcgcctcctcttgctgctgagcccactgccctggctcatctctcccttcaccgctggtccacaaatgtagaccattcttctgtgctgaatattaggacacatggagtgggatttatgtcattcagcagggtggacctcatgtatccaatgagcccatcaccatcccgctgactctgtgagaacattctggttcattttaaactccctcccttgctttctttttcttccagtcatggcaccaaatacgaattaatttgtaatccactagctaagaaattattccaatcctctaaattcctcattttttaaaatcattttgagcctacagaatatccttcccaattaggtacacattatctccctttcaaacatactatttgtctgtcagcctttcagtcattcttttcttttggaaaggtaggaggctgcccctcccgtgctctcccgctttctccctgctctctctttctctcactgttcaagtttgtcagaatttttctattttattagttctttatctttattgtgttcacaaaatatattgtattaaacttttcatttagaaa

SEQ ID NO: 88 - protein, 185 aa, Homo sapiens
MVNSCCGSVCSDQGCGQDLCQETCCCPSCCQTTCCRTTCYRPSYSVSCCCRPQCCQSVCCQPTCCRPSCCVSSCCKPQCCQSVCCQPTCCHPSCCISSCCRPSCCVSSCCKPQCCQSVCCQPNCCRPSCSISSCCRPSCCESSCCRPCCCLRPVCGRVSCHTTCYRPACVISTCPRPVCCASSCC

SEQ ID NO: 89 - DNA, 1100 bp, Homo sapiens
  CDS (1)..(633)
atggtcagctcctgttgtggctccgtgtgctctgaccagggctgcggccaagacctctgtcaggagacctgctgccgccccagctgctgtgagaccacctgctgcaggaccacctgctgccgccccagctgttgtgtatccagctgctgcaggccccagtgctgccagtctgtgtgctgccaacccacttgttcccgccccagctgctgtcagaccacctgttgcaggaccacctgctaccgccccagctgttgtgtgtccagctgctgcaggccccagtgctgccagcctgcgtgctgccaacccacttgctgtcgccccagctgctgtgagacgacctgctgccaccctaggtgctgcatctccagctgctgtcgccccagctgctgtgtgtccagctgctgcaagccccagtgctgccagtctgtgtgctgccagcccaactgctgccgccccagctgcagcatctccagctgctgccgcccctcttgctgtgaatccagctgctgccgcccctgctgctgcgtgcgtccagtctgtggccgagtctcctgccacaccacttgctatcgcccaacctgtgtcatctccagctgcccccgccccttgtgctgtgcctcctcttgctgctgagcccactgccctggcttatctcccccttcaccactggcccacagatgtagacccttctactgtgctgaccattaggatacaggaagtggggttgatgtcattcaatagaatggaactcatgtttccaatgagcccatcaccatttcactgactctttgagaacattctgattcattttaaactccctcccttgctttctttttcttccggtggtggcaccaaatgtgaattaatttgtaatccactagctagaaattattccaatcctctaatttcctcattttctttatcactttgaggtacagatcttcttctcagtgaggcagacattatctgcaggaccagttttgtcactgaggttgcaccctcagatccagccgcccagttatattctgtgtttctcctagtgtgaatttcttatgctttgttgcatctctgctttttaataaaatttctgtacataagaa

SEQ ID NO: 90 - protein, 210 aa, Homo sapiens
MVSSCCGSVCSDQGCGQDLCQETCCRPSCCETTCCRTTCCRPSCCVSSCCRPQCCQSVCCQPTCSRPSCCQTTCCRTTCYRPSCCVSSCCRPQCCQPACCQPTCCRPSCCETTCCHPRCCISSCCRPSCCVSSCCKPQCCQSVCCQPNCCRPSCSISSCCRPSCCESSCCRPCCCVRPVCGRVSCHTTCYRPTCVISSCPRPLCCASSCC

SEQ ID NO: 91 - DNA, 5770 bp, Homo sapiens
  CDS (233)..(4195)
gacaaatgctctggctccggtggtgacaggttgtccagcttcttacagccatcttcactgaaactaaggttaactcctcactctctatggacggctactctctatttccaagggcgtgaagaatttccctcttctcctgtgctttcatctaaaagttctccctggataattatcattgcagtggagtgcctggattggacatcctcatctgggtcaactaaaaaaagaaagcatgcaagacgacagcatagaagcttctacttccatatctcagcttctaagagagagctatttagctgaaaccagacatcggggaaacaatgagaggagtcgagcggagccctcctccaacccttgccatttcggcagtccttctggggccgctgaaggaggcggaggccaagatgaccttccagatctttcagcctttctgagccaagaagaattagacgaaagtgtcaatttggcaagactggccatcaattacgaccctttggagaaggcagatgaaactcaagctagaaaacgactttctcctgatcagatgaaacactcacctaatttaagttttgagcctaacttctgccaggataaccctcgaagtcccaccagctctaaagaaagcccccaggaggcaaaaaggccacagtattgttctgaaacccagtccaaaaaagtatttttaaataaggctgccgacttcattgaagagctatcctcccttttcaaatcccacagctccaaaaggattagacctcgtgcctgcaaaaaccacaagagtaaactggaatctcaaaacaaagttatgcaggaaaacagctccagtttctcagatctgtcagaaagacgagaaagatcttctgttcccatccctatccctgcggataccagggataatgaagtgaatcacgccctggaacagcaggaagccaagaggcgtgaagcggagcaggctgccagtgaggcggctggtggagacactacaccagggtcttccccttcatctctgtactatgaagaacctctggggcaacctccccggttcactcaaaagttacggagcagagaagttccagaaggaactcgagtacagttggattgcatagtggtaggaattccaccacctcaagtaaggtggtactgtgaaggcaaggagcttgaaaattccccagatattcacatcgtccaggcaggaaatctgcactcactgaccattgcggaagcctttgaagaggacacaggacgctattcctgctttgcttctaacatctatgggacagattcgacttctgctgagatttatatagaaggggtttcttcttctgactcagaaggcgaccctaacaaggaagagatgaatcgaatccagaagccaaatgaggtgtcatctcctcccactacctctgcagtcattcctccagcagtaccccaagcccagcatttggtggcccaacctcgtgtggcaaccatccagcagtgtcagagccccaccaattacttgcagggattggatggaaaacctatcattgcagctcctgtgtttacaaagatgctacaaaatttgtcagcttctgagggtcagctggttgtctttgaatgcagagtaaaaggagctccatctcctaaggttgagtggtatagagaagggactttaatagaagattctccagattttaggattttacagaaaaaacctcgatccatggcagagccagaggagatttgcaccttggtcattgctgaggtgtttgcagaagattctgggtgcttcacatgtactgcaagcaacaaatacggcacagtgtcaagcattgcacagctgcacgtgagaggaaatgaggacctcagcaacaacgggtctcttcactcagccaactctaccaccaacctggcagctattgagccacagccctccccaccccactcagagcctccatctgtggaacaaccccccaaacccaaactcgagggggttctggtgaaccacaatgagccccggtccagctccaggattgggcttcgtgtgcacttcaacctgcctgaagatgacaaaggaagtgaagcatcctccgaggctggtgtggtgaccaccagacagaccaggcccgattctttccaggagaggttcaacggacaggcaacaaaaaccccagagccttcttcccccgtgaaagagccccctccagttctggccaaacccaaacttgattccactcagttacaacagcttcataaccaagtcttactggaacaacaccaattgcaaaacccacctccttcatctcctaaggagtttcctttcagcatgactgttttgaactccaatgctcccccagcggtgacaacatccagtaagcaggtgaaggctccttcatcacagacgttcagcttggcccggccgaagtatttcttcccctccacgaacaccaccgcagcaactgtggccccttccagctctccggtgttcactttgagcagcactcctcaaactattcagaggacagtgagcaaagaaagcctcttagtgtctcacccctctgtgcaaaccaaatctccaggagggctttccatccaaaatgagccactcccaccaggcccaacagaaccaacaccaccaccattcacattttccatccccagcggaaaccagtttcagccccgctgtgtgtccccaattcctgtctctcctaccagccggattcagaacccagtggctttcctcagctctgttctgccttctctccctgccatcccacccacaaatgccatggggctgcctagaagtgcaccatccatgccatcccagggattagcgaagaaaaatacaaagtctcctcaaccagtgaatgatgataacattcgtgaaactaagaacgcagtgattcgagacttggggaaaaaaataactttcagtgatgtcagaccaaaccagcaggagtacaaaatttcaagctttgagcagaggctgatgaatgaaatagagtttcgcttggaacgtactcctgttgatgaatcagatgatgaaattcaacatgatgagatccccacgggcaagtgtattgctcccatctttgacaagagactcaagcacttccgggtcacagaaggctctccagtcacattcacctgcaaaattgttgggatacctgttccaaaggtttactggttcaaagatgggaagcagatttctaagagaaatgagcactgcaaaatgaggcgagaaggagatgggacatgctctctgcacattgaatccactaccagtgatgacgatggcaactacaccatcatggcagccaacccccaggggagaatcagctgttctggccacttgatggtacaaagtttgcccattcgcagtcggctaacctctgctggtcagtctcacaggggaagatcccgagtgcaagaaagagacaaagagcccctacaggaacgctttttccgaccacatttcctgcaggctcctggggatatggtagctcatgaggggcgcctctgtcggctggactgtaaggtgagtggtttaccgcccccggagctgacatggctactcaatggccaacctgtgctaccagatgcctcccacaagatgctggtcagggagaccggagtccactctctgctcattgacccactcactcagcgcgacgcagggacctataagtgcatcgctaccaacaaaaccgggcagaattcttttagtctggagctctctgtagtagccaaagaggtgaagaaagcacctgtgatcctggagaaactacagaactgcggtgttcccgaaggccaccccgtgagactggagtgccgcgtgataggcatgcccccacctgtgttctactggaagaaagacaatgagaccatcccttgcaccagagagaggatcagtatgcaccaggacacaacagggtatgcctgccttctcattcagccagccaagaaatcagacgctggatggtacacgttgtcagccaagaatgaagccggcatcgtgtcgtgcactgccaggctggatatatacgctcagtggcaccatcagatcccaccgcccatgtctgtccggcccagtggcagtcgctacggatctctcaccagtaaaggacttgacatattttctgccttttcctccatggaaagcacgatggtgtattcatgctcttctcggagtgtagtggagagtgatgaactttaagaatgtctaggtacctgctgtgtaagagagcggactgtggagggggaatgagaacaagccagacttggtggtttccaagcaaccgaagttgagtaagttcccacactgctggacctgtggcaaagagtgctttgaataagtcagctagggattcttgcagtctcagctgagggagaaaggtagggctgtgccttctaaagattccaacagagatgtgagaagataatacagatgaaccaaagatgtcacacagttgccatccactgctttgggaaaaaactagcatgctcccctgctccctgttgtgttagggatgtttaggtcatactaggagcaattaggagccatatgcctgggctgagaagagctaggcagtagtgaccttaggatatgactaactcaccaaacaatgccaaggagaaaggcggacaggtcaccatcacctttcatcgattacatgtatagcagtagttttggtgaattcaccaaatcagcttccacttagcattaggaggtcatgccatacaactcacgtatgcgggcaaacacttttgatttgcatatcctgggtgtactgagccacataataaggtgtcaaaatgtgcttgagattccaaaaacttgctgaaacacttgatattgcacagtgcacttattttgtggccaaagcatggccttaccagcttgtctgcactattttcttttgggtggtgactgtttttttctgactttgcatatccctagacactagaactaaaggattggtaataaaccaatcagaaagaaatcctctgtggggtcactttaaaaactactagcttcaactaccacttacaaaatgtgcaagagtcatcatttgcccataaaatacacctcatgggctcctacccctttccccaaaagtctgaaagtgtaggaggaaggaagagatacaaacaaggagaggaaatgatgggagagtgttgtttttgtcacttgccccagcagagcaggggttttggaaggagaggtctttaatagctttgaaatgctttgagatcattggtcaaattgcattttctatgtagagaatattctgctaggtggaaaagttgggagaaaggggaatatgcatctttattctaatcaccaattcaaaccctgcctcttaaggaatttttagacttaagttcactgtgatatccctagtacctagaagagtacctagcacaaagtagacattcaataaatatttgctggttgaatgaatcttctgttattatgtctattataagataacctaatcttatattctgcaggaatatgccacccacacaccagtccaaacactgccctgggaatgctcatcacagcacagtttgctctaggctgtgcagggaaatgaattgcaggtgtctgttttcaattccctgtgctggaatccctggtgctgtctgtgccaagcacactcagctggcattgtatttgtgtgaacagacagtaactgcttagaaaggcttgaaactgtcttcacttcaaggcaaggatttccagcataaaaataaattcattcactggagaaaaaaaaaaaaaaaa

SEQ ID NO: 92 - protein, 1320 aa, Homo sapiens
MQDDSIEASTSISQLLRESYLAETRHRGNNERSRAEPSSNPCHFGSPSGAAEGGGGQDDLPDLSAFLSQEELDESVNLARLAINYDPLEKADETQARKRLSPDQMKHSPNLSFEPNFCQDNPRSPTSSKESPQEAKRPQYCSETQSKKVFLNKAADFIEELSSLFKSHSSKRIRPRACKNHKSKLESQNKVMQENSSSFSDLSERRERSSVPIPIPADTRDNEVNHALEQQEAKRREAEQAASEAAGGDTTPGSSPSSLYYEEPLGQPPRFTQKLRSREVPEGTRVQLDCIVVGIPPPQVRWYCEGKELENSPDIHIVQAGNLHSLTIAEAFEEDTGRYSCFASNIYGTDSTSAEIYIEGVSSSDSEGDPNKEEMNRIQKPNEVSSPPTTSAVIPPAVPQAQHLVAQPRVATIQQCQSPTNYLQGLDGKPIIAAPVFTKMLQNLSASEGQLVVFECRVKGAPSPKVEWYREGTLIEDSPDFRILQKKPRSMAEPEEICTLVIAEVFAEDSGCFTCTASNKYGTVSSIAQLHVRGNEDLSNNGSLHSANSTTNLAAIEPQPSPPHSEPPSVEQPPKPKLEGVLVNHNEPRSSSRIGLRVHFNLPEDDKGSEASSEAGVVTTRQTRPDSFQERFNGQATKTPEPSSPVKEPPPVLAKPKLDSTQLQQLHNQVLLEQHQLQNPPPSSPKEFPFSMTVLNSNAPPAVTTSSKQVKAPSSQTFSLARPKYFFPSTNTTAATVAPSSSPVFTLSSTPQTIQRTVSKESLLVSHPSVQTKSPGGLSIQNEPLPPGPTEPTPPPFTFSIPSGNQFQPRCVSPIPVSPTSRIQNPVAFLSSVLPSLPAIPPTNAMGLPRSAPSMPSQGLAKKNTKSPQPVNDDNIRETKNAVIRDLGKKITFSDVRPNQQEYKISSFEQRLMNEIEFRLERTPVDESDDEIQHDEIPTGKCIAPIFDKRLKHFRVTEGSPVTFTCKIVGIPVPKVYWFKDGKQISKRNEHCKMRREGDGTCSLHIESTTSDDDGNYTIMAANPQGRISCSGHLMVQSLPIRSRLTSAGQSHRGRSRVQERDKEPLQERFFRPHFLQAPGDMVAHEGRLCRLDCKVSGLPPPELTWLLNGQPVLPDASHKMLVRETGVHSLLIDPLTQRDAGTYKCIATNKTGQNSFSLELSVVAKEVKKAPVILEKLQNCGVPEGHPVRLECRVIGMPPPVFYWKKDNETIPCTRERISMHQDTTGYACLLIQPAKKSDAGWYTLSAKNEAGIVSCTARLDIYAQWHHQIPPPMSVRPSGSRYGSLTSKGLDIFSAFSSMESTMVYSCSSRSVVESDEL

SEQ ID NO: 93 - DNA, 341 bp, Homo sapiens
atagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatt

SEQ ID NO: 94 - DNA, 3038 bp, Homo sapiens
  CDS (410)..(1876)
ggagtgcccaggatggagtgcagtggctattcacaggtttaatcatagctcactaccgtcttgaatccctgggctcaagtgatcctcctgcctcagcccctgagtagctggtactaaagttaccttccacgctgtatgtccgtgagaaatacaatgaagacggtctgaataatgaactggtaatcttcgataatatgaaacgaatttaagagaacatatcaaagagaaggctctcaaaataggctgctgcaaaagctggtatattgtccggttgctctctcagaatctcgcgtgtcagcccttcaagaagattcccaaatccttgtggaattcggtagtgggtgttggagaatggaatcgacatcttcttggtattggttttcgctttgcttcatacatcaataatgatatggtgctgcagaaggagcctgctggggcagtgatatggggcttcggtacacctggagccacagtgaccgtgaccctgcgccaaggtcaggaaaccatcatgaagaaagtgaccagtgtgaaagctcactctgatacgtggatggtggtactggatcctatgaagcctggaggacctttcgaagtgatggcacaacagactttggagaaaataaacttcaccctgagagttcatgacgtcctgtttggagatgtctggctctgtagtgggcagagtaacatgcagatgactgtgttacagatatttaatgctacaagggagttgtctaacactgcggcatatcagtctgtccgcatcctctctgtctctcccattcaagcagagcaggagctggaggaccttgttgcggttgacttgcagtggtctaagcccacctcagaaaacttaggccatggatatttcaagtacatgtcagcagtgtgctggctctttggacgtcacctttatgacactctgcagtatcccatcgggctgatcgcctccagctggggcgggacacccattgaagcctggtcatctggacggtcactgaaagcctgtggggtccctaaacaagggtccattccatacgattctgtaactggtcccagtaagcactctgttctctggaatgccatgatccatccactgtgcaatatgactctgaaaggggtagtatggtaccagggggagtccaatataaattataacacggatctgtacaattgcacattccctgcactcatcgaagactggcgtgaaaccttccaccgtggttcccaggggcagacggagcgtttcttcccatttggacttgtccagttatcttcagatttgtctaagaagagctcagacgatggatttccccagatccgttggcatcaaacagcagacttcggctatgtccccaacccaaagatgcccaatactttcatggctgtagctatggatctctgtgatagagactcgccttttggcagcatccaccctcgagataaacagactgtggcttatcggctgcatttgggggcccgtgctctggcttatggtgagaagaatttgacctttgaaggaccactgcctgagaagatagaactcttggctcacaaggggctgctcaatctcacatattaccagcaaatccaggtgcagaaaaaggacaacaagatatttgagatctcctgttgcagtgaccatcgatgcaagtggcttccagcttctatgaacaccgtctccacccagtccctgaccctggcgatcgattcttgtcatggcactgtggttgctctccgctatgcttggaccacgtggccttgtgaatataagcagtgtcccctataccaccccagtagtgccctgccagcccctcccttcattgctttcattacagaccagggtcctggacatcagagcaatgttgctaaatgactgtttcagtatgatcagaacttagatataaggatgggtccttcagattttagcatttaggagtttcaataataaccattgcttttaaaggaaattaatagaaagcctcattgaatggctttcagctagcacatggctgtttctatattctgatgagcccaggcttataggtaacttgaaatgcttgctttttgttccctagttggtctaagggtctgtattggactaattctgaactacagacaaattggacctcaatgtcatttacttccctcatattaatgggagtgaaatgtctaatacttttgcccctttttatccagagttgtgggaatctcaggattggaagagattttaaaggccacataggccagctagtgttcatgtgttctttataaaatttctcccatccaagtactaaccaggcccgaccctgcttagcttccgagatcagatgagatcaggcgcgttcagggtgatatggccgtagacgtctttacaaaattcctgacaggtggttactgaatctctctatgaactttccattcaaaactttccaagtttttccttatgtggaaccgaaatctttctttctcccgtgaaactttactactatcagataattgaagacagatctctttgtattctcttcaagcccaaaccaattctgttccttcaatctaaatagtggtaatatgaatgtttaagaaatgaaataagaaacatgtgcaggcactttggaaggtgctaagtgactgccctaaggaatgaaaagcaagggccaggtgggagtagcccagcgaaggcacttgggctgccaggaacaggaggcgtgggaaactctggcttaggaaaacatgaacacaggggcaacagaggcaaactgttgttcgagttaaatataaatctcaggctctttaaaggtaaaaggtttaaggataatccatttggaagaagaaaagagtgaggctgaaagtaaagccacatgacaagcatataaaaaaaaatgcagatgatacaaatatgaaagaggccttcagtgtttgtttattaagaatcttaatgcagtttactgatggattaaaaacagctaacattgtctgaaaattatgttacctataagaagttggaaataaataaaagcataatcactagtgatgtg

SEQ ID NO: 95 - protein, 488 aa, Homo sapiens
MVLQKEPAGAVIWGFGTPGATVTVTLRQGQETIMKKVTSVKAHSDTWMVVLDPMKPGGPFEVMAQQTLEKINFTLRVHDVLFGDVWLCSGQSNMQMTVLQIFNATRELSNTAAYQSVRILSVSPIQAEQELEDLVAVDLQWSKPTSENLGHGYFKYMSAVCWLFGRHLYDTLQYPIGLIASSWGGTPIEAWSSGRSLKACGVPKQGSIPYDSVTGPSKHSVLWNAMIHPLCNMTLKGVVWYQGESNINYNTDLYNCTFPALIEDWRETFHRGSQGQTERFFPFGLVQLSSDLSKKSSDDGFPQIRWHQTADFGYVPNPKMPNTFMAVAMDLCDRDSPFGSIHPRDKQTVAYRLHLGARALAYGEKNLTFEGPLPEKIELLAHKGLLNLTYYQQIQVQKKDNKIFEISCCSDHRCKWLPASMNTVSTQSLTLAIDSCHGTVVALRYAWTTWPCEYKQCPLYHPSSALPAPPFIAFITDQGPGHQSNVAK

SEQ ID NO: 96 - DNA, 20 bp, Artificial
  PCR primer
aaaagcaggttggtcactgg

SEQ ID NO: 97 - DNA, 20 bp, Artificial
  PCR primer
cgacttctgcccattctctc

SEQ ID NO: 98 - DNA, 20 bp, Artificial
  PCR primer
gccaccagctgtcatcacta

SEQ ID NO: 99 - DNA, 20 bp, Artificial
  PCR primer
ccaacaccacaaggaggagt

SEQ ID NO: 100 - DNA, 20 bp, Artificial
  PCR primer
actgtcccggagacaatgac

SEQ ID NO: 101 - DNA, 20 bp, Artificial
  PCR primer
tgctcctaaagccacacctt

SEQ ID NO: 102 - DNA, 20 bp, Artificial
  PCR primer
ccagtctgctctgcctatcc

SEQ ID NO: 103 - DNA, 20 bp, Artificial
  PCR primer
aacgtcaggaactccacacc

SEQ ID NO: 104 - DNA, 20 bp, Artificial
  PCR primer
ccagtccattctgggtccta

SEQ ID NO: 105 - DNA, 20 bp, Artificial
  PCR primer
tggcatgctgttgtatgctt

SEQ ID NO: 106 - DNA, 20 bp, Artificial
  PCR primer
ctgggagcaaacacatctga

SEQ ID NO: 107 - DNA, 20 bp, Artificial
  PCR primer
agttcatgagctgcaacacg

(19) United States
(12) Patent Application Publication
(10) Pub. No.: US 2013/0225443 A1
(43) Pub. Date: Aug. 29, 2013

(54) METHOD OF EXAMINING POLYCYSTIC KIDNEY DISEASE AND METHOD OF SCREENING FOR THERAPEUTIC AGENT OF THE DISEASE

(71) Applicants: Kenji Osafune, Kyoto-shi (JP); Taro Toyoda, Kyoto-shi (JP); Fumihiko Shiota, Kyoto-shi (JP); Kazuwa Nakao, Kyoto-shi (JP); Masakatsu Sone, Kyoto-shi (JP); Daisuke Taura, Kyoto-shi (JP)

(72) Inventors: Kenji Osafune, Kyoto-shi (JP); Taro Toyoda, Kyoto-shi (JP); Fumihiko Shiota, Kyoto-shi (JP); Kazuwa Nakao, Kyoto-shi (JP); Masakatsu Sone, Kyoto-shi (JP); Daisuke Taura, Kyoto-shi (JP)

(73) Assignee: KYOTO UNIVERSITY, Kyoto-shi, Kyoto (JP)

(21) Appl. No.: 13/883,735

(22) PCT Filed: Nov. 7, 2011
(86) PCT No.: PCT/JP2011/006203

§ 371 Date: May 6, 2013

Related U.S. Application Data
(60) Provisional application No. 61/410,757, filed on Nov. 5, 2010.
(60) Provisional application No. 61/530,269, filed on Sep. 1, 2011.

(51) Int. Cl. C07K 16/18; C12N 15/113; C12Q 1/68; C12N 15/11

(52) U.S. Cl. CPC C07K 16/18; C12N 15/11; C12N 15/113; C12Q 1/6883
USPC 506/9; 536/24.31; 530/389.1; 536/24.5; 435/6.12; 435/377; 435/6.11; 435/7.9; 435/7.92

(57) ABSTRACT

The present invention provides a method of examining polycystic kidney disease or a complication of polycystic kidney disease using a gene(s) selected from the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PDCK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE, and a method of screening for a therapeutic agent or a preventive agent therefore, and further vascular endothelial cells or vascular mural cells obtained via differentiation induction from iPS cells formed from a somatic cell of a subject suffered from polycystic kidney disease and having cerebral aneurysm as a complication.

### TECHNICAL FIELD

The present invention relates to a method of examining polycystic kidney disease, a disease marker, and a method of screening for a therapeutic agent for the disease.

### BACKGROUND ART

Polycystic kidney disease is classified into autosomal dominant polycystic kidney disease (ADPKD) and autosomal recessive polycystic kidney disease (ARPKD). ADPKD is developed by about one in 4000 people in Japan. The assumed number of ADPKD patients is said to range from 20,000 to 50,000. ADPKD is causative disease of end-stage chronic renal failure that results in dialysis introduction, and it is the fourth most common disease following diabetic nephropathy, primary glomerular nephritis, and hypertensive nephrosclerosis.

This disease is an autosomal dominant disease due to a genetic mutation of PKD1 or PKD2 (JP Patent Publication (Kohyo) No. 2001-520502 A, JP Patent Publication (Kohyo) No. 2004-504038 A, and JP Patent Publication (Kokai) No. 2009-065988 A). Also, such an autosomal dominant polycystic kidney disease causes a decrease in GLIS3 gene expression level. Hence, it has been reported that the phenomenon has been used for diagnosis of the disease (JP Patent Publication (Kokai) No. 2006-288265 A).

Polycysts are regarded as constituting major kidney pathology. As forms of extrarenal pathology, cyst formation in the liver, pancreas, spleen, generative organ, arachnoid membrane, and the like, cerebral aneurysm and aortic aneurysm, valvular disease of the heart, diverticula of the colon, hernia, and the like have been confirmed. The typical age at onset is middle age, and the age span widely ranges from neonate to 80-year-old.

Although a causative gene of the autosomal dominant polycystic kidney disease has been specified, no effective therapeutic method has been established.

### SUMMARY OF INVENTION

An object of the present invention is to provide a method of examining (or detecting or diagnosing) polycystic kidney disease or a complication that accompanies polycystic kidney disease through comparison of disease markers using samples from subjects, and disease markers of the disease.

Furthermore, an object of the present invention is to provide a method of screening for a drug useful for preventing or treating polycystic kidney disease or a complication that accompanies polycystic kidney disease using the disease markers, and to provide a drug or a medicine useful for treating the disease.

As used herein, the “polycystic kidney disease” includes autosomal dominant polycystic kidney disease (ADPKD) and autosomal recessive polycystic kidney disease (ARPKD). Preferably ADPKD.

The present invention has the following features.

[1] A method of examining whether or not a subject has polycystic kidney disease or a risk of developing the disease, comprising the following steps of:

(a) measuring the expression levels of 1 or more genes selected from or all genes of the group consisting of NTNG1, POSTN, TNC, KAL1, BST1, ACAT2, INSIG1, SCD, HSD3B1, KRT7, USP40, and SULT1E1 in a sample from a subject; and

(b) determining that the subject has polycystic kidney disease or a risk of developing the disease when the expression levels of 1 or more genes selected from or all genes of the group consisting of the NTNG1, POSTN, TNC, KAL1, BST1, HSD3B1, KRT7, USP40, and SULT1E1 are lower than those in a control sample, or, determining that the subject has polycystic kidney disease or a risk of developing the disease when the expression levels of ACAT2, INSIG1, or SCD or the three genes are higher than those in the control sample.

[2] The method according to [1], wherein the sample is at least one type of sample selected from the group consisting of blood, serum, blood plasma, cell extracts, urine, lymph fluids, tissue fluids, ascites, spinal fluids, and other body fluids, or tissues and cells.

[3] The method according to [1], wherein the sample is a vascular endothelial cell obtained via differentiation induction from an iPS cell formed from an isolated somatic cell of a subject, and the gene in step (a) is selected from the group consisting of NTNG1, POSTN, TNC, KAL1, BST1, ACAT2, INSIG1, and SCD.

[4] The method according to [1], wherein the sample is a vascular mural cell obtained via differentiation induction from an iPS cell formed from an isolated somatic cell of a subject, and the gene in step (a) is selected from the group consisting of HSD3B1, KRT7, USP40, and SULT1E1.

[5] The method according to any of [1] to [4], wherein the step of measuring the expression level of the gene is a step of measuring the amount of mRNA, cRNA, or cDNA of the gene or the amount of a protein encoded by the gene in the sample.

[6] A method of examining whether or not a subject has a complication of polycystic kidney disease or a risk of developing the complication, comprising the following steps of:

(a) measuring the expression levels of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE in a sample from a subject with polycystic kidney disease; and

(b) determining that the subject has a complication of polycystic kidney disease or a risk of developing the complication when the expression levels of 1 or more genes selected from or all genes of the group consisting of NTNG1, POSTN, TNC, KAL1, BST1, HSD3B1, KRT7, USP40, and SULT1E1 are lower than those in a control sample, or, determining that a subject has a complication of polycystic kidney disease or a risk of developing the complication when the expression levels of 1 or more genes selected from or all genes of the group consisting of ACAT2, INSIG1, SCD, BMP6, CD274, CTGF, E2F7, EDN¹, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE are higher than those in the control sample.

[7] The method according to [6], wherein the complication is cerebral aneurysm.

[8] The method according to [6] or [7], wherein the sample is at least one type of sample selected from the group consisting of blood, serum, blood plasma, cell extracts, urine, lymph fluids, tissue fluids, ascites, spinal fluids, and other body fluids, or tissues and cells.

[9] The method according to [6] or [7], wherein the sample is a vascular endothelial cell obtained via differentiation induction from an iPS cell formed from an isolated somatic cell of a subject; and wherein the gene in step (a) is 1 or more genes selected from or all genes of the group consisting of NTNG1, POSTN, TNC, KAL1, BST1, ACAT2, INSIG1, SCD, BMP6, CD274, CTGF, E2F7, EDN¹, FAM43A, FRMD3, MMP10, MYEOV, NR2F1, NRCAM, PCSK1, PLXNA2, SLC30A3, SNAI1, SPOCD1, MMP1, and TFPI2.

[10] The method according to [6] or [7], wherein the sample is a vascular mural cell obtained via differentiation induction from an iPS cell formed from an isolated somatic cell of a subject; and wherein the gene in step (a) is 1 or more genes selected from or all genes of the group consisting of HSD3B1, KRT7, USP40, SULT1E1, MMP1, TFPI2, HMGA2, KRTAP4-7, KRTAP4-8, KRTAP4-9, MYPN, RPPH1, and SIAE.

[11] The method according to any of [6] to [10], wherein the step of measuring the expression level of the gene is a step of measuring the amount of the mRNA, cRNA, or cDNA of the gene or the amount of the protein encoded by the gene.

[12] A disease marker for examining polycystic kidney disease or a complication of polycystic kidney disease, comprising: a polynucleotide(s) and/or a polynucleotide(s) complementary thereto, which has/have at least 15 continuous nucleotides in the nucleotide sequence(s) of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or an antibody (or antibodies) or a fragment(s) thereof, which recognizes a protein(s) encoded by 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

[13] The disease marker according to [12], wherein the complication is cerebral aneurysm.

[14] The disease marker according to [13], comprising: a polynucleotide(s) and/or a polynucleotide(s) complementary thereto, which has/have at least 15 continuous nucleotides in the nucleotide sequence(s) of 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or, an antibody (or antibodies) or a fragment(s) thereof, which recognizes a protein(s) encoded by 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

[15] A kit for examining polycystic kidney disease or a complication of polycystic kidney disease, comprising:

a polynucleotide(s) and/or a polynucleotide(s) complementary thereto, which has/have at least 15 continuous nucleotides in the nucleotide sequence(s) of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or an antibody (or antibodies) or a fragment(s) thereof, which recognizes a protein(s) encoded by 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

[16] The kit according to [15], wherein the complication is cerebral aneurysm.

[17] The kit according to [16], comprising:

a polynucleotide(s) and/or a polynucleotide(s) complementary thereto, which has/have at least 15 continuous nucleotides in the nucleotide sequence(s) of 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or

an antibody (or antibodies) or a fragment(s) thereof, which recognizes a protein(s) encoded by 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

[18] A method of screening for a therapeutic agent or a preventive agent for polycystic kidney disease, comprising the following steps of:

(a) bringing each of candidate substances into contact with a vascular endothelial cell obtained via differentiation induction from an iPS cell formed from an isolated somatic cell of a subject with polycystic kidney disease;

(b) measuring the expression levels or the transcriptional activity of 1 or more genes selected from or all genes of the group consisting of NTNG1, POSTN, TNC, KAL1, BST1, ACAT2, INSIG1, and SCD; and

(c) selecting a candidate substance as a therapeutic agent or a preventive agent for polycystic kidney disease, when the expression levels or the transcriptional activity of 1 or more genes selected from or all genes of the group consisting of NTNG1, POSTN, TNC, KAL1, and BST1 exhibit an increase compared with a case in which the candidate substance is not brought into contact, or,

when the expression levels or the transcriptional activity of 1 or more genes selected from or all genes of the group consisting of ACAT2, INSIG1, and SCD exhibit a decrease compared with a case in which the candidate substance is not brought into contact.

[19] A method of screening for a therapeutic agent or a preventive agent for polycystic kidney disease, comprising the following steps of:

(a) bringing each of candidate substances into contact with a vascular mural cell obtained via differentiation induction from an iPS cell formed from an isolated somatic cell of a subject with polycystic kidney disease;

(b) measuring the expression levels or the transcriptional activity of 1 or more genes selected from or all genes of the group consisting of HSD3B 1, KRT7, USP40, and SULT1E1; and

(c) selecting a candidate substance as a therapeutic agent or a preventive agent for polycystic kidney disease, when the expression levels or the transcriptional activity exhibit an increase compared with a case in which the candidate substance is not brought into contact.

[20] The screening method according to [18] or [19], wherein the step of measuring the expression level of the gene is a step of measuring the amount of mRNA, cRNA, or cDNA of the gene.

[21] A method of screening for a therapeutic agent or a preventive agent for a complication of polycystic kidney disease, comprising the following steps of:

(a) bringing each of candidate substances into contact with a vascular endothelial cell obtained via differentiation induction from an iPS cell formed from an isolated somatic cell of a subject with a complication of polycystic kidney disease;

(b) measuring the expression levels or the transcriptional activity of 1 or more genes selected from or all genes of the group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2; and

(c) selecting a candidate substance as a therapeutic agent or a preventive agent for a complication of polycystic kidney disease, when the expression levels or the transcriptional activity of 1 or more genes selected from or all genes of the group consisting of NTNG1, POSTN, TNC, KAL1, and BST1 exhibit an increase compared with a case in which the candidate substance is not brought into contact, or when the expression levels or the transcriptional activity of 1 or more genes selected from or all genes of the group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibit a decrease compared with a case in which the candidate substance is not brought into contact.

[22] A method of screening for a therapeutic agent or a preventive agent for a complication of polycystic kidney disease, comprising the following steps of:

(a) bringing each of candidate substances into contact with a vascular mural cell obtained via differentiation induction from an iPS cell formed from an isolated somatic cell of a patient with a complication of polycystic kidney disease;

(b) measuring the expression levels or the transcriptional activity of 1 or more genes selected from or all genes of the group consisting of HSD3B 1, KRT7, USP40, SULT1E1, MMP1, TFPI2, HMGA2, KRTAP4-7, KRTAP4-8, KRTAP4-9, MYPN, RPPH1, and SIAE; and

(c) selecting a candidate substance as a therapeutic agent or a preventive agent for a complication of polycystic kidney disease, when the expression levels or the transcriptional activity of 1 or more genes selected from or all genes of the group consisting of HSD3B1, KRT7, USP40, and SULT1E1 exhibit an increase compared with a case in which the candidate substance is not brought into contact, or, when the expression levels or the transcriptional activity of 1 or more genes selected from or all genes of the group consisting of MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

[23] The screening method according to [21] and [22], wherein the complication is cerebral aneurysm.

[24] The screening method according to [21] and [22], wherein the step of measuring the expression level of the gene is a step of measuring the amount of mRNA, cRNA, or cDNA of the gene.

[25] A composition for treating or preventing polycystic kidney disease or a complication of polycystic kidney disease, comprising a pharmaceutically effective amount(s) of an antisense oligonucleotide(s), or siRNA(s) or shRNA(s) against at least one nucleic acid selected from the group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE genes or transcription products thereof.

[26] The composition according to [25], wherein the complication is cerebral aneurysm.

[27] The composition according to [26], comprising a pharmaceutically effective amount(s) of an antisense oligonucleotide(s), or siRNA(s) or shRNA(s) against at least one nucleic acid selected from the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE genes or transcription products thereof.

[28] A vascular endothelial cell obtained via differentiation induction from an iPS cell formed from an isolated somatic cell of a subject suffered from polycystic kidney disease and having cerebral aneurysm as a complication, wherein the expression levels of NTNG1, POSTN, TNC, KAL1, and BST1 are lower and the expression levels of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in a vascular endothelial cell obtained via differentiation induction from an iPS cell formed from an isolated somatic cell of a healthy subject.

[29] A vascular endothelial cell obtained via differentiation induction from an iPS cell formed from an isolated somatic cell of a subject suffered from polycystic kidney disease and having cerebral aneurysm as a complication, wherein the expression levels of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are high.

[30] A vascular mural cell obtained via differentiation induction from an iPS cell formed from an isolated somatic cell of a subject suffered from polycystic kidney disease and having cerebral aneurysm as a complication, wherein the expression levels of HSD3B1, KRT7, USP40, and SULT1E1 are lower and the expression levels of MMP1, TFPI2, HMGA2, KRTAP4-7, KRTAP4-8, KRTAP4-9, MYPN, RPPH1, and SIAE are higher than those in a vascular mural cell obtained via differentiation induction from an iPS cell formed from an isolated somatic cell of a healthy subject.

[31] A vascular mural cell obtained via differentiation induction from an iPS cell formed from an isolated somatic cell of a subject suffered from polycystic kidney disease and having cerebral aneurysm as a complication, wherein the expression levels of MMP1, TFPI2, HMGA2, KRTAP4-7, KRTAP4-8, KRTAP4-9, MYPN, RPPH1, and SIAE are high.

According to the method of the present invention, examination of polycystic kidney disease or a complication that accompanies polycystic kidney disease (hereinafter, simply referred to as “a complication of polycystic kidney disease” or “a polycystic kidney disease complication”), and preparation of a disease marker for the disease, screening for a drug useful for preventing or treating the disease, and preparation of a drug useful for treating the diseases can be carried out.

### BRIEF DESCRIPTION OF DRAWINGS

FIG. 1 shows the results of RT-PCR for the indicated genes (FIG. 1A: SCD, NTNG1, POSTN, TNC, KAL1; FIG. 1B: INSIG1 (V1 and 2), BST1) in vascular endothelial cells obtained via differentiation induction from iPS cells formed from fibroblast cells of Japanese persons (nonPK) not developing autosomal dominant polycystic kidney disease (PK) and of autosomal dominant polycystic kidney disease (PK) patients with cerebral aneurysm.

FIG. 2 shows the results of RT-PCR for the indicated genes (i.e., HSD3B1, KRT7, USP40, SULT1E1) in vascular mural cells obtained via differentiation induction from iPS cells formed from fibroblast cells of nonPK persons and PK patients.

FIG. 3 shows the results of quantitative PCR (i.e., expression levels) for the indicated genes (i.e., A, CD274; B, CTGF; C, MMP10; D, NRCAM; E, MMP1) in iPS cells or iPS cell-derived vascular endothelial cells or iPS cell-derived vascular mural cells, which cells were directly or indirectly prepared from fibroblast cells of autosomal dominant polycystic kidney disease patients with cerebral aneurysm (PK-ane-iPS1) or without cerebral aneurysm (PK-free-iPS3).

### DESCRIPTION OF EMBODIMENTS

### <Polynucleotides as Disease Markers>

The present invention is based on the following finding. The presence or the absence or the degree of an increase (rise) and/or a decrease (fall) in the expression level of at least one of genes listed in Table 1 compared with control samples is measured (qualitative and/or quantitative measurement), so that the onset of polycystic kidney disease or a complication of polycystic kidney disease or the risk of developing the disease can be specifically detected and thus the disease can be examined precisely.

Specifically, the present invention provides a disease marker useful as a tool with which the presence or the absence of the onset of or the degree of polycystic kidney disease or a complication of polycystic kidney disease can be examined through measurement of the presence or the absence of an increase and/or a decrease in or the degree of the expression of the above gene in a subject.

As used herein, the term of “polycystic kidney disease” means both of autosomal dominant polycystic kidney disease and autosomal recessive polycystic kidney disease unless otherwise mentioned.

As used herein, the term “subject” refers to a mammalian animal, which includes, but is not limited to, primate, rodent, ungulate or the like, preferably human. The subject may be used exchangeably with another term “patient.”

In the present invention, examples of a complication of polycystic kidney disease include vascular lesions such as aortic aneurysm, cerebral aneurysm, or subarachnoid hemorrhage, valvular disease of heart, diverticula of colon, hernia, and ductal lesions such as choledochal dilatation, as well as symptoms such as cyst formation in the liver, pancreas, spleen, and generative organs. An example of such a complication that should be particularly preferably detected is cerebral aneurysm.

TABLE 1

| | | |
|---|---|---|
| Gene | GenBank | SEQ ID NO: |

| Name | Accession No. | Gene | Protein |
|---|---|---|---|
| NTNG1 | NM_001113226 | 1 | 2 |
| POSTN | NM_001135934 | 3 | 4 |
| TNC | NM_002160 | 5 | 6 |
| KAL1 | NM_000216 | 7 | 8 |
| BST1 | NM_004334 | 9 | 10 |
| INSIG1 | NM_005542 | 11 | 12 |
| SCD | NM_005063 | 13 | 14 |
| HSD3B1 | NM_000862 | 15 | 16 |
| KRT7 | NM_005556 | 17 | 18 |
| USP40 | NM_018218 | 19 | 20 |
| SULT1E1 | NM_005420 | 21 | 22 |
| ACAT2 | NM_005891 | 45 | 46 |
| BMP6 | NM_001718 | 47 | 48 |
| CD274 | NM_014143 | 49 | 50 |
| CTGF | NM_001901 | 51 | 52 |
| E2F7 | NM_203394 | 53 | 54 |
| EDN1 | NM_001955 | 55 | 56 |
| FAM43A | NM_153690 | 57 | 58 |
| FRMD3 | NM_174938 | 59 | 60 |
| MMP10 | NM_002425 | 61 | 62 |
| MYEOV | NM_138768 | 63 | 64 |
| NR2F1 | NM_005654 | 65 | 66 |
| NRCAM | NM_001037132 | 67 | 68 |
| PCSK1 | NM_000439 | 69 | 70 |
| PLXNA2 | NM_025179 | 71 | 72 |
| SLC30A3 | NM_003459 | 73 | 74 |
| SNAI1 | NM_005985 | 75 | 76 |
| SPOCD1 | NM_144569 | 77 | 78 |
| MMP1 | NM_001145938 | 79 | 80 |
| TFPI2 | NM_006528 | 81 | 82 |
| HMGA2 | NM_001145938 | 83 | 84 |
| KRTAP4-7 | NM_033061 | 85 | 86 |
| KRTAP4-8 | NM_031960 | 87 | 88 |
| KRTAP4-9 | NM_001146041 | 89 | 90 |
| MYPN | NM_032578 | 91 | 92 |
| RPPH1 | NR_002312 | 93 | — |
| SIAE | NM_001199922 | 94 | 95 |

The disease marker of the present invention is characterized by comprising a polynucleotide and/or a polynucleotide complementary thereto, which has at least 15 continuous nucleotides in an open reading frame (ORF) sequence in the nucleotide sequences of the genes listed in Table 1. Persons skilled in the art can easily understand that the following sequences are contained as transcriptional mutants or splicing mutants in the genes listed in Table 1 and examples are not particularly limited thereto. Specifically, in Table 1, NTNG1 comprises the sequence according to accession No. NM_{—}001113228 or NM_{—}014917, POSTN comprises the sequence according to accession No. NM_{—}001135935, NM_{—}001135936, or NM_{—}006475, INSIG1 comprises the sequence according to accession No. NM_{—}198336 or NM_{—}198337, EDN1 comprises the sequence according to accession No. NM_{—}001168319, NRCAM comprises the sequence according to accession No. NM_{—}001193582, NM_{—}001193583, NM_{—}001193584, or NM_{—}005010, PCSK1 comprises the sequence according to accession No. NM_{—}001177875 or NM_{—}001177876, MMP1 comprises the sequence according to accession No. NM_{—}002421, HMGA2 comprises the sequence according to accession No. NM_{—}002421, and SIAE comprises the sequence according to accession No. NM_{—}170601.

The term “polynucleotide complementary thereto (complementary strand or reverse strand) as used herein refers to a polynucleotide that is in a basically complementary relationship (on the basis of a base pair relationship such as A:T and G:C) with: an ORF sequence of the nucleotide sequence shown in SEQ ID NO: 1, 3, 5, 7, 9, 11, 13, 15, 17, 19, 21, 45, 47, 49, 51, 53, 55, 57, 59, 61, 63, 65, 67, 69, 71, 73, 75, 77, 79, 81, 83, 85, 87, 89, 91, 93, or 94; or a sequence (partial sequence) (for the sake of convenience, these ORF sequence and partial sequence are also referred to as “forward strands”) having an at least continuous 15-nucleotide-long nucleotide sequence in the ORF sequence. However, such a complementary strand may not only be a complete complementary sequence to the nucleotide sequence of a target forward strand, but also be a sequence having a complementary relationship with the other to a degree such that it can hybridize to a target forward strand under stringent conditions. In addition, stringent conditions can be determined based on the melting temperature (Tm) of a nucleic acid to which a complex or a probe is bound, as taught by Berger and Kimmel (1987, Guide to Molecular Cloning Techniques Methods in Enzymology, Vol. 152, Academic Press, San Diego Calif.). Examples of washing conditions after hybridization generally include conditions of about 1×SSC, 0.1% SDS, and 37 degrees C. A complementary strand to be used herein is preferably capable of maintaining its state of hybridizing to a target forward strand even when washed under such conditions. Examples of more stringent hybridization conditions include, but are not particularly limited to, conditions that allow a forward strand and a complementary strand to be able to maintain the hybridization state even when they are washed under washing conditions of about 0.5×SSC, 0.1% SDS, and 42 degrees C. or more stringent washing conditions of 0.1×SSC, 0.1% SDS, and 65 degrees C. Specific examples of such a complementary strand include a strand comprising a nucleotide sequence that is in a completely complementary relationship with the nucleotide sequence of a target forward strand, and a strand comprising a nucleotide sequence having at least 90%, and preferably at least 95%, 96%, 97%, 98%, or 99% sequence identity with the strand.

Also, examples of a polynucleotide on the forward strand side include not only an ORF sequence of the nucleotide sequence according to SEQ ID NO: 1, 3, 5, 7, 9, 11, 13, 15, 17, 19, 21, 45, 47, 49, 51, 53, 55, 57, 59, 61, 63, 65, 67, 69, 71, 73, 75, 77, 79, 81, 83, 85, 87, 89, 91, 93, or 94, or a partial sequence thereof, but also a strand comprising a nucleotide sequence that is in a further complementary relationship with the nucleotide sequence of the above forward strand.

The above forward-strand polynucleotide and complementary-strand (reverse-strand) polynucleotide may be separately used as disease markers in a single-strand form or a double-strand form.

As described above, the disease marker for polycystic kidney disease or a complication of polycystic kidney disease of the present invention may be a polynucleotide comprising a partial sequence of the above ORF sequence or a sequence complementary thereto as long as it selectively (or specifically) recognizes a gene(s) listed in Table 1 or a polynucleotide from the gene. In this case, examples of such a polynucleotide include polynucleotides having a length of at least 15, at least 18, at least 19, at least 20, at least 30, at least 40, at least 50, at least 60, at least 70, or at least 100 continuous nucleotides, which is arbitrarily selected from the nucleotide sequence of the above ORF sequence or a sequence complementary thereto.

In addition, as used herein, the term “selectively (or specifically) recognizes” means, but is not limited to: that when a Northern blot method or a Southern blot method is employed for example, genes listed in Table 1 or polynucleotides from the genes can be specifically detected; or that when an RT-PCR method is employed, the genes listed in Table 1 or polynucleotides formed from the genes are specifically amplified and generated, as long as persons skilled in the art can determine that detected products in the Northern blot method or the Southern blot method or products in the above RT-PCR method are derived from the genes listed in Table 1 or polynucleotides originating therefrom.

Such a disease marker of the present invention can be designed based on the nucleotide sequence of the gene shown in SEQ ID NO: 1, 3, 5, 7, 9, 11, 13, 15, 17, 19, 21, 45, 47, 49, 51, 53, 55, 57, 59, 61, 63, 65, 67, 69, 71, 73, 75, 77, 79, 81, 83, 85, 87, 89, 91, 93, or 94, using primer 3 (http//www.genome.wi.mit.edu/cgi-bin/primer/primer3.cgi) or vector NTI (Infomax), for example.

Specifically, candidate sequences of primers or probes, which are obtained by subjecting the nucleotide sequences of the genes listed in Table 1 to primer 3 or vector NTI software, or sequences containing at least the sequence as a portion can be used as primers or probes.

A disease marker to be used in the present invention may have a length of at least 15 continuous nucleotides, at least 18 continuous nucleotides, at least 19 continuous nucleotides, at least 20 continuous nucleotides, at least 30 continuous nucleotides, at least 40 continuous nucleotides, or at least 50 continuous nucleotides, at least 60 continuous nucleotides, at least 70 continuous nucleotides, or at least 100 continuous nucleotides, as described above. Specifically, the length can be appropriately selected and determined depending on the applications of the marker.

The disease marker of the present invention can be used as a primer for specifically recognizing and amplifying RNA resulting from the expression and/or transcription of the gene or a polynucleotide (e.g., cDNA) from the RNA, or can be used as a probe for specifically detecting the RNA or a polynucleotide (e.g., cDNA) from the RNA. When the above disease marker is used as a primer for examining or detecting polycystic kidney disease or a complication of polycystic kidney disease, an example thereof is a disease marker with a nucleotide length generally ranging from 15 bp to 100 bp, preferably ranging from 15 bp to 50 bp, or more preferably ranging from 20 bp to 35 bp. Also, when the disease marker is used as a detection probe, an example thereof is a disease marker with a nucleotide length ranging from generally 15 bp to the full-length sequence, preferably ranging from 15 bp to 1 kb, or more preferably ranging from 50 bp to 500 bp.

When the disease marker of the present invention is used as a primer, preferable primer sets are exemplified in Table 2.

TABLE 2
Table 2

| | | SEQ |
|---|---|---|
| | | ID |
| Primer Name^{*)} | Sequence | NO: |
| NTNG1-primer-F | CCCCTGAGCTGATGTTTGAT | 23 |
| NTNG1-primer-R | GACGCCCAGATTCAAAGGTA | 24 |
| POSTN-primer-F | GCAGACACACCTGTTGGAAA | 25 |
| POSTN-primer-R | GTCACGGGGATTTCTTTGAA | 26 |
| TNC-primer-F | GTCACCGTGTCAACCTGATG | 27 |
| TNC-primer-R | GCCTGCCTTCAAGATTTCTG | 28 |
| KAL1 primer-F | GGCTGAGGTCACTACGGAAA | 29 |
| KAL1-primer-R | GGTCTCAGATTGGGCACTGT | 30 |
| BST1-primer-F | GTGCTGCCCTCAGACTATGACC | 31 |
| BST1-primer-R | CTGCCATACAGAACATCGCTCAG | 32 |
| INSIG1-primer-F | GGTGGACATTTGATCGTTCC | 33 |
| INSIG1-primer-R | TGACGCCTCCTGAGAAAAAT | 34 |
| SCD-primer-F | TGGTTTCACTTGGAGCTGTG | 35 |
| SCD-primer-R | GCCATGCAATCAATGAAGAA | 36 |
| HSD3B1-primer-F | AGGACGTCTCGGTCATCATC | 37 |
| HSD3B1-primer-R | CTGGCACACTAGCTTGGACA | 38 |
| KRT7-primer-F | CAGGAACTCATGAGCGTGAA | 39 |
| KRT7-primer-R | GATATTCACGGCTCCCACTC | 40 |
| USP40-primer-F | GGGGCACCGTATTACTTGAA | 41 |
| USP40-primer-R | GGCTTCTTGGCTTTTCCTTC | 42 |
| SULT1E1-primer-F | CAGAAATTGTCGCCCTTCAT | 43 |
| SULT1E1-primer-R | GATTCCTTCATTTGCTGCTCA | 44 |
| ACAT2-primer-F | AAAAGCAGGTTGGTCACTGG | 96 |
| ACAT2-primer-R | CGACTTCTGCCCATTCTCTC | 97 |
| CD274-primer-F | GCCACCAGCTGTCATCACTA | 98 |
| CD274-primer-R | CCAACACCACAAGGAGGAGT | 99 |
| CTGF-primer-F | ACTGTCCCGGAGACAATGAC | 100 |
| CTGF-primer-R | TGCTCCTAAAGCCACACCTT | 101 |
| MMP10-primer-F | CCAGTCTGCTCTGCCTATCC | 102 |
| MMP10-primer-R | AACGTCAGGAACTCCACACC | 103 |
| NRCAM-primer-F | CCAGTCCATTCTGGGTCCTA | 104 |
| NRCAM-primer-R | TGGCATGCTGTTGTATGCTT | 105 |
| MMP1-primer-F | CTGGGAGCAAACACATCTGA | 106 |
| MMP1-primer-R | AGTTCATGAGCTGCAACACG | 107 |

^{*)}F indicates a forward primer. R indicates a reverse primer.

When the disease marker of the present invention is used as a probe, the probe may be labeled with a radioisotope (e.g., ³²P or ³³P), a fluorescent substance (fluorescamine, rhodamine, Texas Red, Dansyl, or a derivative thereof), a chemiluminescence substance, an enzyme, or the like. Such a labeled disease marker can be appropriately used as a probe (or a detection marker).

The disease marker of the present invention can be used as a primer or a probe according to conventional methods including known methods for specifically recognizing or detecting a specific gene, mRNA, and cDNA, such as a Northern blot method, a Southern blot method, RT-PCR, and in situ hybridization.

In the present invention, examination of polycystic kidney disease or a complication of polycystic kidney disease (which may also be referred to as “detection” or “diagnosis”) can be performed by measuring the presence or the absence of an increase or a decrease in or the level (the degree of increase or decrease in expression) of the expression of a gene(s) (listed in Table 1) in a sample obtained from at least 1 type of sample selected from the group consisting of subject-derived blood, serum, blood plasma, cell extracts, urine, lymph fluids, tissue fluids, ascites, spinal fluids, and other body fluids, or tissues and cells (e.g., renal tissues or renal cells, and somatic cells obtained via differentiation induction from iPS cells), and evaluating the thus obtained results.

In an aspect of the present invention, the present invention provides a method of examining whether or not a subject has polycystic kidney disease or a complication of polycystic kidney disease or a risk of developing the disease, comprising the following steps of:

(a) measuring the expression levels of 1 or more genes selected from or all genes of a group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE in a sample from a subject; and

(b) determining that the subject has polycystic kidney disease or a risk of developing the disease when the expression levels of 1 or more genes selected from or all genes of a group consisting of NTNG1, POSTN, TNC, KAL1, BST1, HSD3B1, KRT7, USP40, and SULT1E1 are lower than those in a control sample, or that the subject has polycystic kidney disease or a risk of developing the disease when the expression level of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE are higher than those in the control sample.

In an embodiment of the present invention, vascular endothelial cells obtained via differentiation induction in vitro from iPS cells formed from somatic cells isolated from a subject may be used as samples in the method of examining (detecting or diagnosing) polycystic kidney disease or a complication of polycystic kidney disease according to the present invention. In this case, the method can be performed by measuring the presence or the absence of a decrease in or the level (the degree of decrease in expression) of the expression of 1 or more genes selected from or all genes of a group consisting of NTNG1, POSTN, TNC, KAL1, and BST1 in Table 1 and then evaluating the thus obtained results, and/or the method can be performed by measuring the presence or the absence of increase in or the level (degree of increase in expression) of the expression of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 in Table 1, and then evaluating the thus obtained results. Specifically, the method comprises the steps of: inducing iPS cells from somatic cells isolated from a subject to cause differentiation induction of vascular endothelial cells from the iPS cells; measuring the expression levels or the transcriptional activity of 1 or more genes selected from or all genes of a group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 in vascular endothelial cells; and determining that the subject has polycystic kidney disease or a complication of polycystic kidney disease or a risk of developing the disease when the expression levels or the transcriptional activity of 1 or more genes selected from or all genes of a group consisting of NTNG1, POSTN, TNC, KAL1, and BST1 exhibit a decrease (or reduction) compared with a control sample (that is, vascular endothelial cells obtained via differentiation induction from iPS cells formed from somatic cells of a healthy subject), or, that the subject has polycystic kidney disease or a complication of polycystic kidney disease or a risk of developing the disease when the expression levels or the transcriptional activity of 1 or more genes selected from or all genes of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibit an increase (or enhancement) compared with the control sample.

In another embodiment, vascular mural cells obtained via differentiation induction from iPS cells formed from somatic cells of a subject may also be used as samples for the examination (detection or diagnosis) of polycystic kidney disease or a complication of polycystic kidney disease according to the present invention. In this case, the method can be performed by measuring the presence or the absence of decrease in or the level (the degree of decrease in expression) of the expression of 1 or more genes selected from or all genes of a group consisting of HSD3B1, KRT7, USP40, and SULT1E1 in Table 1, and then evaluating the thus obtained results, and/or measuring the presence or the absence of increase in or the level (the degree of increase in expression) of the expression of 1 or more genes selected from or all genes of the group consisting of MMP1, TFPI2, HMGA2, KRTAP4-7, KRTAP4-8, KRTAP4-9, MYPN, RPPH1, and SIAE in Table 1, and then evaluating the thus obtained results. Specifically, the method comprises the steps of: inducing iPS cells from somatic cells of a subject to cause differentiation induction of vascular mural cells from the iPS cells; measuring the expression levels or the transcriptional activity of 1 or more genes selected from or all genes of a group consisting of HSD3B1, KRT7, USP40, and SULT1E1 in the vascular mural cells; and determining that the subject has polycystic kidney disease or a complication of polycystic kidney disease or a risk of developing the disease when the expression levels or the transcriptional activity of 1 or more genes selected from or all genes of a group consisting of HSD3B1, KRT7, USP40, and SULT1E1 exhibit a decrease (or reduction) compared with a control sample (that is, vascular mural cells obtained via differentiation induction from iPS cells formed from somatic cells of a healthy subject), or that the subject has polycystic kidney disease or a complication of polycystic kidney disease, or a risk of developing the disease when the expression levels or the transcriptional activity of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibit an increase (or enhancement) compared with the control sample.

### <Antibodies as Disease Markers>

The present invention further provides an antibody capable of specifically recognizing the expression product (protein) of a gene(s) listed in Table 1 as a disease marker for polycystic kidney disease or a complication of polycystic kidney disease.

An example of a protein encoded by a gene(s) listed in Table 1 is a protein encoded by the polynucleotide shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, or 94. A preferable embodiment is a protein having the amino acid sequence shown in SEQ ID NO: 2, 4, 6, 8, 10, 12, 14, 16, 18, 20, 22, 46, 48, 50, 52, 54, 56, 58, 60, 62, 64, 66, 68, 70, 72, 74, 76, 78, 80, 82, 84, 86, 88, 90, 92, or 95.

The form of the antibody of the present invention is not particularly limited and may be a polyclonal or monoclonal antibody, which can be prepared using a protein(s) listed in Table 1 as an immunizing antigen, a chimeric antibody (e.g., a human/mouse chimeric antibody), a humanized antibody, a human antibody, or the like, or, a fragment (e.g., Fab, Fab′, F(ab′)₂, Fc, Fv, and scFv) of such an antibody. Moreover, an antibody having an antigen binding ability for a polypeptide comprising at least 8 continuous amino acids (e.g., 10 to 20 amino acids) in the amino acid sequence of the protein is also included in the examples of the antibody of the present invention. Methods for producing such antibodies are known. The antibody of the present invention can also be produced according to these conventional methods (Current protocols in Molecular Biology edit. Ausubel et al. (1987) Publish. John Wiley and Sons. Section 11.12-11. 13).

Specifically, when the antibody of the present invention is a polyclonal antibody, a non-human animal such as a rabbit is immunized with the protein encoded by a gene listed in Table 1, expressed in Escherichia coli or the like, and then purified according to conventional methods or an oligo peptide synthesized having a partial amino acid sequence of the protein. Thus, the polyclonal antibody can be obtained from the serum of the immunized animal according to conventional methods.

Meanwhile, when the antibody of the present invention is a monoclonal antibody, it can be obtained from hybridoma cells prepared by fusing the thus obtained spleen cells and myeloma cells (obtained from the above-immunized non-human animal) through HAT selection and affinity assay with a target polypeptide, for example (Current protocols in Molecular Biology edit. Ausubel et al., (1987) Publish. John Wiley and Sons. Section 11.4-11. 11).

The protein to be used for preparation of an antibody can be obtained by DNA cloning, construction of each plasmid, transfection into a host, culture of a transformant, and subsequent collection of the protein from a culture product based on the gene sequence information provided by the present invention. These procedures can be carried out according to methods known by persons skilled in the art or methods described in documents (Molecular Cloning, T. Maniatis et al., CSH Laboratory (1983), DNA Cloning, D M. Glover, IRL PRESS (1985)), for example. Specifically, a recombinant DNA (or an expression vector) that enables expression of a gene in desired host cells is prepared, the DNA is introduced into host cells for transformation, the transformant is cultured, and then a target protein is collected from the thus obtained culture product, so that the protein can be obtained. Also, the protein can also be produced by general chemical synthesis methods (peptide synthesis) according to the amino acid sequence information provided by the present invention, as another technique.

In addition, examples of the proteins encoded by genes listed in Table 1 of the present invention include not only a protein having the amino acid sequence shown in SEQ ID NO: 2, 4, 6, 8, 10, 12, 14, 16, 18, 20, 22, 46, 48, 50, 52, 54, 56, 58, 60, 62, 64, 66, 68, 70, 72, 74, 76, 78, 80, 82, 84, 86, 88, 90, 92, or 95, but also a portion homologous thereto. An example of such a homologous portion is a protein comprising an amino acid sequence that has deletion, substitution, or addition of 1 or a plurality of amino acids, preferably 1 or several amino acids, with respect to each amino acid sequence shown in SEQ ID NO: above, or, an amino acid sequence having at least 90%, preferably at least 95%, 96%, or 97%, further preferably at least 98%, and most preferably at least 99% sequence identity with each amino acid sequence shown in SEQ ID NO: above, and, having biological functions equivalent to and/or immunological activity equivalent to that of the protein having each amino acid sequence shown in SEQ ID NO: above. Such a homologous portion contains a mutant on the basis of racial polymorphism, mutation, splice mutation, and the like.

In addition, an example of a protein having biological functions equivalent to the known functions of a protein having the amino acid sequence shown in SEQ ID NO: 2, 4, 6, 8, 10, 12, 14, 16, 18, 20, 22, 46, 48, 50, 52, 54, 56, 58, 60, 62, 64, 66, 68, 70, 72, 74, 76, 78, 80, 82, 84, 86, 88, 90, 92, or 95 is a protein having biochemical or pharmacological functions equivalent to the same. Also, an example of such a protein having immunological activity equivalent to that of the above protein is a protein capable of specifically binding to an antibody against the above protein.

The term “sequence identity” as used herein can be defined as a percentage (%) of the number of identical amino acid residues or identical nucleotides of total number of amino acid residues or nucleotides (both cases include the number of gaps) when two amino acid sequences or two nucleotide sequences are aligned to achieve the maximum match of amino acids or nucleotides with or without introduction of gaps. The sequence identity can be determined using BLAST (Altschul S F, et al, (1997) Nucleic Acids Res. 25 (17): 3389-402 or (1990) J Mol. Biol. 215(3): 403-10) that can be found from the NCBI server, ncbi.nlm.nih.gov/BLAST/.

Furthermore, the number of amino acid mutations or mutation sites in proteins is not limited, as long as their biological functions and/or immunological activity is retained. An indicator for determining the way of substituting, inserting, or deleting amino acid residues and the number thereof without losing biological functions or immunological activity can be found using a computer program known to persons skilled in the art, such as DNA Star software. For example, the number of mutations typically accounts for 10% or less of all amino acids, preferably 5% or less of all amino acids, and further preferably accounts for 1% or less of all amino acids. Also, amino acids to be substituted are not particularly limited, as long as proteins obtained after substitution with the relevant amino acids have biological functions and/or immunological activity equivalent to that of the original protein. Preferably, in view of the retention of protein configuration, amino acids to be substituted have properties such as electrical properties and structural properties (e.g., polarity, electric charge, solubility, hydrophobicity, hydrophilicity, and amphiphilicity of amino acid residues) analogous to those of unsubstituted amino acids. For example, Ala, Val, Leu, Ile, Pro, Met, Phe, and Trp are amino acids that are classified as nonpolar amino acids from each other. Gly, Ser, Thr, Cys, Tyr, Asn, and Gln are amino acids that are classified as uncharged amino acids. Asp and Glu are amino acids that are classified as acidic amino acids. Further, Lys, Arg, and H is are amino acids that are classified as basic amino acids. Therefore, amino acids to be substituted can be appropriately selected from among amino acids belonging to the same group using these amino acid properties as indicators.

Also, the antibody of the present invention may be prepared using a polypeptide

(intended to include an oligo peptide) having a partial amino acid sequence of a protein encoded by a gene(s) listed in Table 1 (see above). A polypeptide to be used for inducing such an antibody is not required to have functional bioactivity, but desirably has immunogenicity analogous to that of the protein. A preferable example of a polypeptide to be used herein has such immunogenicity and comprises at least 8 continuous amino acid residues (e.g., 10 to 20 amino acid residues) in the amino acid sequence of SEQ ID NO: 2, 4, 6, 8, 10, 12, 14, 16, 18, 20, 22, 46, 48, 50, 52, 54, 56, 58, 60, 62, 64, 66, 68, 70, 72, 74, 76, 78, 80, 82, 84, 86, 88, 90, 92, or 95.

An antibody against such a polypeptide can also be prepared by enhancing immunological responses with the use of various adjuvants depending on host animals. Examples of such an adjuvant include, but are not limited to: Freund's adjuvant; and mineral gel such as aluminum hydroxide; as well as surface active substances such as lysolecithin, pluronic polyols, polyanions, peptides, oil emulsions, keyhole limpet hemocyanin, and dinitrophenol; and human adjuvants such as BCG (bacillus Caluuette Guecin) and Corynebacterium-parvum.

The antibody of the present invention has a property of specifically binding to a protein encoded by a gene(s) listed in Table 1. Hence, through the use of such an antibody, the above protein contained in a sample from a subject can be specifically detected and quantitatively determined. Specifically, the antibody of the present invention is useful for examining (or detecting or diagnosing) polycystic kidney disease or a complication of polycystic kidney disease.

### <Method of Examining Polycystic Kidney Disease or a Complication of Polycystic Kidney Disease>

The present invention provides a method comprising the following steps, (a-1) and (b-1), as a method of examining polycystic kidney disease:

(a-1) measuring the expression levels of a protein encoded by 1 or more genes selected from or all genes of a group consisting of NTNG1, POSTN, TNC, KAL1, BST1, ACAT2, INSIG1, SCD, HSD3B1, KRT7, USP40, and SULT1E1 in a sample from a subject; and

(b-1) determining that the subject has polycystic kidney disease or a risk of developing the disease when the expression levels of the proteins encoded by 1 or more genes selected from or all genes of a group consisting of NTNG1, POSTN, TNC, KAL1, BST1, HSD3B1, KRT7, USP40, and SULT1E1 are lower than those in a healthy subject-derived control sample, or determining that the subject has polycystic kidney disease or a risk of developing the disease when the expression level of 1 or more genes selected from or all genes of a group consisting of ACAT2, INSIG1, and SCD are higher than those in a healthy subject-derived control sample.

Similarly, a method comprising the following steps, (a-2) and (b-2), is provided as the method of examining a complication of polycystic kidney disease:

(a-2) measuring the expression levels of 1 or more genes selected from or all genes of a group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE in a sample from a subject with polycystic kidney disease; and

(b-2) determining that the subject has a complication of polycystic kidney disease or a risk of developing the complication when the expression levels of 1 or more genes selected from or all genes of a group consisting of NTNG1, POSTN, TNC, KAL1, BST1, HSD3B1, KRT7, USP40, and SULT1E1 are lower than those in a healthy subject-derived control sample, or determining that the subject has a complication of the same or a risk of developing the complication when the expression levels of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE are higher than those in a healthy subject-derived control sample.

Similarly, a method comprising the following steps, (a-3) and (b-3), is provided as the method of examining cerebral aneurysm which is a complication:

(a-3) measuring the expression levels of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE in a sample from a subject with polycystic kidney disease; and

(b-3) determining that the subject has cerebral aneurysm or a risk of developing the disease when the expression levels of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE are higher than those in a healthy subject-derived control sample.

Here, as samples from a subject or control samples from a healthy subject, blood, serum, blood plasma, cell extracts, urine, lymph fluids, tissue fluids, ascites, spinal fluids, or other body fluids, or tissues or cells (e.g., renal tissues or renal cells or somatic cells obtained via differentiation induction from iPS cells) can be used. As such somatic cells obtained via differentiation induction from the iPS cells formed from somatic cells of a subject, tubular cells, collecting tubule cells, bile duct cells, hepatocytes, pancreatic ductal cells, pancreatic cells, bowel cells, germ cells, vascular endothelial cells or vascular mural cells, and preferably, vascular endothelial cells or vascular mural cells can be used, for example. Control samples to be used herein are corresponding somatic cells obtained via differentiation induction from iPS cells formed from somatic cells of a healthy subject. The healthy subject-derived somatic cells for induction of iPS cells may differ from or may be of the same type as subject-derived somatic cells. Examples of such somatic cells are as described later in the section of “method of producing iPS cells.” Also, in this connection, “corresponding somatic cells” means that somatic cells obtained via differentiation induction from iPS cells formed from healthy subject-derived somatic cells are cells of the same type as that of somatic cells obtained via differentiation induction from iPS cells formed from subject-derived somatic cells.

When vascular endothelial cells obtained via in vitro differentiation induction from iPS cells formed from isolated subject-derived somatic cells is used as a sample from a subject, the present invention further provides a method comprising the following steps, (a-4) and (b-4), as the method of examining polycystic kidney disease:

(a-4) measuring the expression levels of 1 or more genes selected from or all genes of a group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 or the expression levels of a protein(s) encoded by the gene(s) in vascular endothelial cells obtained via differentiation induction from iPS cells formed from subject-derived somatic cells; and

(b-4) determining that the subject has polycystic kidney disease or a risk of developing the disease when the expression levels of 1 or more genes selected from or all genes of a group consisting of NTNG1, POSTN, TNC, KAL1, and BST1 or the expression levels of the protein(s) encoded by the gene(s) are lower than those in a healthy subject-derived control sample, or, determining that the subject has polycystic kidney disease or a risk of developing the disease when the expression levels of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in a healthy subject-derived control sample.

In another aspect, the present invention further provides a method comprising the following steps, (a-5) and (b-5), as the method of examining polycystic kidney disease, when vascular mural cells obtained via in vitro differentiation induction from iPS cells formed from isolated subject-derived somatic cells are used as a sample from a subject:

(a-5) measuring the expression levels of 1 or more genes selected from or all genes of a group consisting of HSD3B1, KRT7, USP40, SULT1E1, MMP1, TFPI2, HMGA2, KRTAP4-7, KRTAP4-8, KRTAP4-9, MYPN, RPPH1, and SIAE or the expression levels of a protein(s) encoded by the gene(s) in vascular mural cells obtained via differentiation induction from iPS cells formed from subject-derived somatic cells; and

(b-5) determining that the subject has polycystic kidney disease or a risk of developing the disease when the expression levels of 1 or more genes selected from or all genes of a group consisting of HSD3B1, KRT7, USP40, and SULT1E1 or the expression level(s) of the protein(s) encoded by the gene(s) are lower than those in vascular mural cells (control sample) obtained via differentiation induction from iPS cells formed from a healthy subject-derived somatic cells, or determining that the subject has polycystic kidney disease or a risk of developing the disease when the expression levels of 1 or more genes selected from or all genes of a group consisting of MMP1, TFPI2, HMGA2, KRTAP4-7, KRTAP4-8, KRTAP4-9, MYPN, RPPH1, and SIAE are higher than those in a healthy subject-derived control sample.

In another aspect, the present invention further provides a method comprising the following steps, (a-6) and (b-6), as the method of examining a complication of polycystic kidney disease when vascular endothelial cells obtained via differentiation induction from iPS cells formed from subject-derived somatic cells are used as a sample from a subject:

(a-6) measuring the expression levels of 1 or more genes selected from or all genes of a group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 or the expression levels of the protein(s) encoded by the gene(s) in vascular endothelial cells obtained via differentiation induction from iPS cells formed from somatic cells of a subject with polycystic kidney disease; and

(b-6) determining that the subject has a complication of polycystic kidney disease or a risk of developing the complication when the expression levels of 1 or more genes selected from or all genes of a group consisting of NTNG1, POSTN, TNC, KAL1, and BST1 or the expression levels of the protein(s) encoded by the gene(s) are lower than those in vascular endothelial cells (control sample) obtained via differentiation induction from iPS cells formed from healthy subject-derived somatic cells, or determining that the subject has a complication of polycystic kidney disease or a risk of developing the complication when the expression levels of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in the control sample.

A method comprising the following steps, (a-7) and (b-7), is provided as the method of examining a complication of cerebral aneurysm when vascular endothelial cells obtained via differentiation induction from iPS cells formed from somatic cells of a subject with polycystic kidney disease are used:

(a-7) measuring the expression levels of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 or the expression levels of a protein(s) encoded by the gene(s) in vascular endothelial cells obtained via differentiation induction from iPS cells formed from somatic cells of a subject with polycystic kidney disease; and

(b-7) determining that the subject has cerebral aneurysm or a risk of developing the disease when the expression levels of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in the control sample.

In another aspect, the present invention further provides a method comprising the following steps, (a-8) and (b-8), as the method of examining a complication of polycystic kidney disease when vascular mural cells obtained via differentiation induction from iPS cells formed from somatic cells from a subject are used as a sample from a subject:

(a-8) measuring the expression levels of 1 or more genes selected from or all genes of a group consisting of HSD3B1, KRT7, USP40, SULT1E1, MMP1, TFPI2, HMGA2, KRTAP4-7, KRTAP4-8, KRTAP4-9, MYPN, RPPH1, and SIAE or the expression levels of a protein(s) encoded by the gene(s) in vascular mural cells obtained via differentiation induction from iPS cells formed from somatic cells of a subject with polycystic kidney disease; and

(b-8) determining that the subject has a complication of polycystic kidney disease when the expression levels of 1 or more genes selected from or all genes of a group consisting of HSD3B1, KRT7, USP40, and SULT1E1 or the expression levels of the protein(s) encoded by the gene(s) are lower than those in vascular mural cells (control sample) obtained via differentiation induction from iPS cells formed from somatic cells of a healthy subject, or, determining that the subject has a complication of polycystic kidney disease or a risk of developing the disease when the expression levels of 1 or more genes selected from or all genes of a group consisting of MMP1, TFPI2, HMGA2, KRTAP4-7, KRTAP4-8, KRTAP4-9, MYPN, RPPH1, and SIAE are higher than those in the control sample.

A method comprising the following steps, (a-9) and (b-9), is provided as a more preferable method of examining a complication of cerebral aneurysm when vascular mural cells obtained via differentiation induction from iPS cells formed from somatic cells of a subject with polycystic kidney disease are used:

(a-9) measuring the expression levels of 1 or more genes selected from or all genes of a group consisting of MMP1, TFPI2, HMGA2, KRTAP4-7, KRTAP4-8, KRTAP4-9, MYPN, RPPH 1, and SIAE or the expression levels of a protein(s) encoded by the gene(s) in vascular mural cells obtained via differentiation induction from iPS cells formed from somatic cells of a subject with polycystic kidney disease; and

(b-9) determining that the subject has cerebral aneurysm or a risk of developing the disease when the expression levels of 1 or more genes selected from or all genes of a group consisting of MMP1, TFPI2, HMGA2, KRTAP4-7, KRTAP4-8, KRTAP4-9, MYPN, RPPH1, and SIAE are higher than those in the control sample.

The term “control” as used herein preferably refers to, unless otherwise specified, a measurement result obtained by a step similar to the above from a healthy subject not affected by polycystic kidney disease.

Here, blood, serum, blood plasma, cell extracts, urine, lymph fluids, tissue fluids, ascites, spinal fluids, and other body fluids, or, somatic cells obtained via differentiation induction from iPS cells formed from somatic cells of a subject (e.g., tubular cells, collecting tubule cells, bile duct cells, hepatocytes, pancreatic ductal cells, pancreatic cells, bowel cells, germ cells, vascular endothelial cells, and vascular mural cells) may be directly used. More preferable examples thereof include subject-derived blood, serum, blood plasma, cell extracts, urine, lymph fluids, tissue fluids, ascites, spinal fluids, and other body fluids, or, mRNA prepared by a conventional method from the above cells obtained via differentiation induction from subject-derived iPS cells or a polynucleotide further prepared from such mRNA (e.g., cDNA and cRNA), proteins, or extracts containing protein-containing fractions. Here, methods for producing tubular cells, collecting tubule cells, bile duct cells, hepatocytes, pancreatic ductal cells, pancreatic cells, bowel cells, germ cells, vascular endothelial cells, or vascular mural cells from iPS cells are not particularly limited. These cells can be obtained by appropriately extracting from the embryoid bodies or formed teratomas (e.g., JP Patent Publication (Kokai) No. 2006-239169 A). Hepatocytes can be prepared by a method described in WO2006/082890, JP Patent Publication (Kokai) No. 2010-75631 A or Hay D C, et al, Proc Natl Acad Sci U.S.A., 105, 12301-6 2008, but the examples thereof are not particularly limited thereto. Similarly, pancreatic cells can be prepared using the method described in WO2007/103282. iPS cells as well as vascular endothelial cells or vascular mural cells can be produced by methods described later.

The examination method of the present invention can be specifically performed as described below depending on the types of biological samples to be subjected to measurement.

When mRNA or a polynucleotide prepared therefrom (e.g., cDNA or cRNA) is used as a subject to be measured, the following steps can be used as the above steps (i) and (ii):

(i) binding a mRNA prepared from a sample from a subject or a complementary polynucleotide transcribed from the mRNA, to the above disease marker; and

(ii) measuring RNA from a biological sample or a complementary polynucleotide (cDNA) transcribed from the RNA, which has bound to the disease marker, using the existing amount of the above disease marker as an indicator.

When mRNA or a polynucleotide prepared therefrom (e.g., cDNA or cRNA) (hereinafter, referred to as “mRNA” or the like) is used as a sample to be used for detection, the examination method of the present invention is carried out by detecting and measuring the degree of expression or the expression level of a gene(s) listed in Table 1 such as the mRNA. Specifically, the measurement of mRNA can be carried out by a known method, such as the Northern blot method, the Southern blot method, RT-PCR, quantative PCR, DNA chip analysis, or in situ hybridization analysis, using the disease markers comprising the above polynucleotides of the present invention as primers or probes.

When the Northern blot method or the Southern blot method is used, the expression level of a target gene in mRNA or the like can be detected or measured using the above disease marker of the present invention as a probe. A specific example thereof comprises labeling the disease marker of the present invention (or complementary strand in the case of RNA) with a radioisotope (RI, e.g., ³²P or ³³P, a fluorescent substance, or the like, performing hybridization of the labeled disease marker to mRNA or the like from a living tissue of a subject, which has been transferred to a nylon membrane or the like according to conventional methods, and then detecting or measuring the thus formed double strand of the disease marker and mRNA or the like through detection of signals from the label (e.g., RI or a fluorescent substance) for the disease marker using a radiation detector (Typhoon FLA 9000, GE HEALTHCARE) or fluorescence detector. Alternatively, a method that can be used herein comprises labeling a disease marker according to protocols attached to AlkPhos Direct Labeling and Detection System (Amersham Pharmacia Biotech), performing hybridization of the labeled disease marker to mRNA or the like from a living tissue of a subject, and then detecting or measuring signals from the label of the disease marker using a MultiBio Imager STORM 860 (Amersham Pharmacia Biotech), for example. When the RT-PCR method is used, the expression levels of the genes listed in Table 1 in RNA or the like can be detected or measured using the above disease markers of the present invention as primers. A specific example of the method comprises preparing cDNA according to conventional methods from RNA from a living tissue of a subject, performing PCR according to a conventional method using the resulting cDNA as a template and the disease markers of the present invention as primers, so that a target gene region can be amplified, and detecting the thus obtained amplified doublestranded DNA.

PCR comprises performing approximately 20-40 cycles, each consisting of a denaturation step, an annealing step, and an extension step, for example. The denaturation step is to denature and dissociate double-stranded DNA into single-stranded DNAs at generally about 94-98 degrees C. for about 10 sec to about 2 min). The annealing step is to perform annealing a sense primer or an antisense primer to each single-stranded DNA as a template at generally about 50-68 degrees C. for about 10 sec to 1 min. The extension step is to extend primers along the template DNA, which is generally performed at about 72 degrees C. for about 20 sec to 10 min, for example. Before the above cycles are performed, pretreatment may be performed for doublestranded DNA under conditions similar to the conditions for denaturation. Also, after completion of the above cycles, posttreatment may be performed under conditions similar to the conditions for extension. For PCR, PCR buffer and heat stable DNA polymerase are used. Amplification products can be confirmed by electrophoresis, for example. PCR can be performed using a commercial PCR apparatus such as a thermal cycler.

Furthermore, when DNA chip analysis is employed, an example thereof is a method that comprises preparing a DNA chip in which the disease marker of the present invention as a DNA probe (either single-stranded or double-stranded polynucleotide) attached to the surface, causing cRNA (prepared by a conventional method from RNA derived from a living tissue of a subject) to hybridize to the DNA chip, binding the formed double strand of DNA and cRNA to the disease marker of the present invention as a labeled probe (separately labeled with RI, a fluorescent substance, or the like), and then performing detection. An example of such a DNA chip with which the expression level of a gene can be detected or measured as described above is a Gene Chip (Affymetrix).

When a protein is used as a subject to be measured, an example of a method to be employed herein comprises bringing a protein into contact with an antibody against the disease marker of the present invention, and then detecting or quantitatively determining the protein or a partial peptide thereof binding to the antibody by a known detection method such as the Western blot method or ELISA using the disease marker of the present invention as an indicator.

The Western blot method can be carried out by: after the use of the above antibody against the disease marker of the present invention as a primary antibody, labeling a complex of a protein or a partial peptide thereof and the disease marker (primary antibody) with the use of a secondary antibody (the antibody labeled with a radioisotope such as ¹²⁵I, an enzyme such as horseradish peroxidase (HRP), or a fluorescent substance, which is capable of binding to the primary antibody), and then detecting and measuring signals from the radioisotope or the fluorescent substance using a radiation meter (Typhoon FLA 9000, GE HEALTHCARE) or fluorescence detector. Alternatively, after an antibody against the above disease marker of the present invention is used as a primary antibody, detection can be performed according to protocols attached to the ECL Plus Western Blotting Detection System (Amersham Pharmacia Biotech) and then measurement can be performed using Multi Bio Imager STORM860 (Amersham Pharmacia Biotech).

When subject-derived blood, serum, blood plasma, cell extracts, urine, lymph fluids, tissue fluids, ascites, spinal fluids, or other body fluids exemplified above, or tissues or cells are used as samples, immunoassay can be performed. Examples of such a method include radioimmunoassay, enzyme immunoassay, fluorescence immunoassay, luminescent immunoassay, immunoprecipitation, immunonephelometry, Western blot, and immunodiffusion. A preferable example thereof is enzyme immunoassay and a particularly preferable example thereof is enzyme-linked immunosorbent assay (ELISA) (e.g., sandwich ELISA). The above immunological methods such as ELISA can be performed by methods known by persons skilled in the art. Specifically, ELISA is performed as follows. A solution containing an antibody against the disease marker of the present invention is added as a primary antibody to a support such as a plate, so as to immobilize the antibody. After washing of the plate, blocking is performed using BSA or the like in order to prevent non-specific binding of proteins. The plate is washed again, and then a sample is added to the plate. After incubation, washing is performed and then a labeled antibody such as a biotin-labeled antibody is added as a secondary antibody. After appropriate incubation, the plate is washed and then avidin bound to an enzyme such as alkaline phosphatase or peroxidase is added. After incubation, the plate is washed, a substrate corresponding to the enzyme binding to avidin is added, and then the amount of a desired protein is detected using an enzymatic change or the like of the substrate as an indicator.

Autosomal dominant polycystic kidney disease or a complication of polycystic kidney disease can be examined (or detected or diagnosed) by, preferably:

comparing the expression level of a gene listed in Table 1 (above) or a protein encoded by the gene in a sample from a subject (the expression level is reflected by the amount of binding of a polynucleotide as the disease marker of the present invention with a probe for examination, or the amount of binding of a protein or a partial peptide as the disease marker of the present invention with an antibody for examination or a fragment thereof, as measured above) with the expression level of the gene or the protein in a sample (as a control to be similarly measured) from a healthy subject who is not affected by polycystic kidney disease;

and performing examination based on differences between the two. In this case, if the expression level of a target gene or protein in a subject is 1.5 or more times, 2 or more times, 3 or more times, preferably 5 or more times, and further preferably 10 or more times lower or higher than that of a healthy subject, whether or not the subject has the disease can be confirmed with even higher reliability.

In addition, comparison between a sample from a subject and a control sample from a healthy subject for the expression level of a target gene or the protein can be performed by performing measurements in parallel. If the measurements are not performed in parallel, an average or median of the expression levels obtained by measuring a plurality of (at least 2, preferably 3 or more, and more preferably 5 or more) controls under substantially the same measurement conditions can be used for comparison as a basic level.

### <Method of Producing iPS Cells>

iPS cells to be used in the present invention can be prepared by introducing a specific nuclear reprogramming factor (also, referred to as “reprogramming factor”) in the form of DNA or protein into somatic cells or increasing the expression of the endogenous mRNA or protein of the nuclear reprogramming factor using a drug (K. Takahashi and S. Yamanaka (2006) Cell, 126: 663-676, K. Takahashi et al. (2007) Cell, 131: 861-872, J. Yu et al. (2007) Science, 318: 1917-1920, M. Nakagawa et al. (2008) Nat. Biotechnol., 26: 101-106, International Publication WO 2007/069666, and International Publication WO 2010/068955). A nuclear reprogramming factor may be a gene specifically expressed in ES cells, a gene playing an important role in maintenance of undifferentiation of ES cells, or a gene product thereof. Examples of such nuclear reprogramming factor include, but are not particularly limited to, Oct3/4, Klf4, Klf1, Klf2, Klf5, Sox2, Sox1, Sox3, Sox15, Sox17, Sox18, c-Myc, L-Myc, NMyc, TERT, SV40 Large T antigen, HPV16 E6, HPV16 E7, Bmil, Lin28, Lin28b, Nanog, Esrrb, Esrrg, and Glis1. These reprogramming factors may be used in combination upon establishment of iPS cells. Such combination may contain at least one, two, or three reprogramming factors above and preferably contains 3 or 4 reprogramming factors above.

The nucleotide sequence information of the mouse and human cDNAs of each of the above nuclear reprogramming factors and the amino acid sequence information of proteins encoded by the cDNAs can be obtained by referring to NCBI accession numbers described in WO 2007/069666. Also, the mouse and human cDNA sequence and amino acid sequence information of L-Myc, Lin28, Lin28b, Esrrb, Esrrg, and Glis 1 can be each obtained by referring to the following NCBI accession numbers. Persons skilled in the art can prepare desired nuclear reprogramming factors by a conventional technique based on the cDNA sequence or amino acid sequence information.

Gene Name Mouse Human

L-Myc NM_{—}008506 NM_{—}001033081

Lin28 NM_{—}145833 NM_{—}024674

Lin28b NM_{—}001031772 NM_{—}001004317

Esrrb NM_{—}011934 NM_{—}004452

Esrrg NM_{—}011935 NM_{—}001438

Glis1 NM_{—}147221 NM_{—}147193

These nuclear reprogramming factors may be introduced in the form of protein into somatic cells by a technique such as lipofection, binding with a cell membranepermeable peptide, or microinjection. Alternatively, they can also be introduced in the form of DNA into somatic cells by a technique such as a technique using a vector (e.g., a virus, a plasmid, or an artificial chromosome), lipofection, a technique using a liposome, or microinjection.

Examples of the viral vector include retrovirus vector, lentivirus vector (see Cell, 126, pp. 663-676, 2006; Cell, 131, pp. 861-872, 2007; and Science, 318, pp. 1917-1920, 2007), adenovirus vector (see Science, 322, 945-949, 2008), adeno-associated virus vector, and Sendai virus vector (see Proc Jpn Acad Ser B Phys Biol Sci. 85, 348-62, 2009).

Also, examples of the artificial chromosome vector include human artificial chromosome (HAC), yeast artificial chromosome (YAC), and bacterial artificial chromosome (BAC and PAC).

As the plasmid, a plasmid for mammalian cells can be used (see Science, 322: 949-953, 2008).

The above vector can contain regulatory sequences such as a promoter, an enhancer, a ribosome binding sequence, a terminator, and a polyadenylation site, so that a nuclear reprogramming factor can be expressed. Examples of the promoter to be used herein include EF1 alpha promoter, CAG promoter, SR alpha promoter, SV40 promoter, LTR promoter, CMV (cytomegalovirus) promoter, RSV (Rous sarcoma virus) promoter, MoMuLV (Moloney mouse leukemia virus) LTR, and HSV-TK (herpes simplex virus thymidine kinase) promoter. Particularly, examples thereof include EF1 alpha promoter, CAG promoter, MoMuLV LTR, CMV promoter, and SR alpha promoter. The vector may further contain, if necessary, a selection marker sequence such as a drug resistance gene (e.g., a kanamycin resistance gene, an ampicillin resistance gene, and a puromycin resistance gene), a thymidine kinase gene, and a diphtheria toxin gene, and a reporter gene sequence such as a green fluorescent protein (GFP), beta glucuronidase (GUS), and FLAG.

Also, in order to cleave both a gene encoding a nuclear reprogramming factor or a promoter and a gene encoding a nuclear reprogramming factor binding thereto after introduction into somatic cells, the above vector may have LoxP sequences located before and after the relevant portions. In another preferred embodiment, a method comprising incorporating a transgene into a chromosome using transposon, causing transferase to act on cells using a plasmid vector or an adenovirus vector, and then completely removing the transgene from the chromosome can be employed. An example of preferable transposon is piggyBac or the like that is lepidopteran insectderived transposon (Kaji, K. et al., (2009), Nature, 458: 771-775, Woltjen et al., (2009), Nature, 458: 766-770, WO 2010/012077).

Furthermore, the above vector may also contain sequences of replication origins of lymphotrophic herpes virus, BK virus, and Bovine papilloma virus and sequences relating to the replication thereof so that they can be episomally present as a result of replication even if they are not incorporated into a chromosome. For example, the vector contains EBNA-1 and oriP or Large T and SV40ori sequences (WO 2009/115295, WO 2009/157201, and WO 2009/149233).

Furthermore, for simultaneous introduction of a plurality of nuclear reprogramming factors, an expression vector for polycistronic expression may also be used. For polycistronic expression, sequences of genes may be connected to each other by intervening IRES or a foot and mouth disease virus (FMDV) 2A coding region between them (Science, 322: 949-953, 2008; WO 2009/092042; and WO 2009/152529). Upon nuclear reprogramming, to improve the efficiency for induction of iPS cells, in addition to the above factors or elements, histone deacetylase (HDAC) inhibitors [e.g., low-molecular weight inhibitors such as valproic acid (VPA) (Nat. Biotechnol., 26(7): 795-797 (2008)), trichostatin A, sodium butyrate, MC 1293, and M344, and nucleic acid expression inhibitors such as siRNA and shRNA against HDAC (e.g., HDAC1 siRNA Smartpool (Registered Trademark) (Millipore) and HuSH 29mer shRNA Constructs against HDAC1 (OriGene))], DNA methyltransferase inhibitors (e.g., 5′-azacytidine) (Nat. Biotechnol., 26 (7): 795-797 (2008)), G9a histone methyltransferase inhibitors [e.g., low-molecular-weight inhibitors such as BIX-01294 (Cell Stem Cell, 2: 525-528 (2008)) and nucleic acid expression inhibitors such as siRNA and shRNA against G9a (e.g., G9a siRNA (human) (Santa Cruz Biotechnology))], L-channel calcium agonists (e.g., Bayk8644) (Cell Stem Cell, 3, 568-574 (2008)), p53 inhibitors (e.g., siRNA and shRNA against p53) (Cell Stem Cell, 3, 475-479 (2008)), Wnt Signalling activator (e.g., soluble Wnt3a) (Cell Stem Cell, 3, 132-135 (2008)), growth factors such as LIF or bFGF, ALKS inhibitors (e.g., SB431542) (Nat Methods, 6: 805-8 (2009)), mitogen-activated protein kinase signaling inhibitors, glycogen synthase kinase-3 inhibitors (PloS Biology, 6(10), 2237-2247 (2008)), miRNA such as miR-291-3p, miR-294, and miR-295 (R. L. Judson et al., Nat. Biotechnol., 27: 459-461 (2009)), for example, can be used.

Examples of a drug to be used in a method for increasing the expression of an endogenous protein of a nuclear reprogramming factor include 6-bromoindirubin-3′-oxime, indirubin-5-nitro-3′-oxime, valproic acid, 2-(3-(6-methylpyridin-2-yl)-1H-pyrazol-4-yl)-1,5-naphthyridine, 1-(4-methylphenyl)-2-(4,5,6,7-tetrahydro-2-imino-3(2H)-benzothiazolyl)ethanone HBr(pifithrin-alpha), prostaglandin J2, and prostaglandin E2 (WO 2010/068955). Examples of a culture medium for inducing iPS cells include (1) a DMEM, DMEM/F12, or DME medium containing 10-15% FBS (these media may further appropriately contain LIF, penicillin/streptomycin, puromycin, L-glutamine, nonessential amino acids, Beta-mercaptoethanol, and the like), (2) a medium for ES cell culture containing bFGF or SCF, such as a medium for mouse ES cell culture (e.g., TX-WES medium (Thromb-X)), and a medium for primate ES cell culture (e.g., a medium for primate (human & monkey) ES cells, ReproCELL, Kyoto, Japan (mTeSR-1)).

An example of culture methods is as follows. Somatic cells are brought into contact with nuclear reprogramming factors (nucleic acids or proteins) in a DMEM or DMEM/F12 medium containing 10% FBS at 37 degrees C. in the presence of 5% CO₂ and are cultured for about 4 to 7 days. Subsequently, the cells are reseeded on feeder cells (e.g., mitomycin C-treated STO cells or SNL cells). About 10 days after contact between the somatic cells and the nuclear reprogramming factors, cells are cultured in a bFGF-containing medium for primate ES cell culture. About 30-45 days or more after the contact, ES cell-like colonies can be formed. Cells may also be cultured under conditions in which the oxygen concentration is as low as 5-10% in order to increase the efficiency for induction of iPS cells.

Alternatively, cells may be cultured using DMEM containing 10-% FBS (which may further appropriately contain LIF, penicillin/streptomycin, puromycin, L-glutamine, nonessential amino acids, beta-mercaptoethanol, and the like) on feeder cells (e.g., mitomycin C-treated STO cells or SNL cells). After about 25-30 days or more, ES cell-like colonies can be formed.

During the above culture, medium exchange with a fresh medium is performed once a day from day 2 after the start of culture. In addition, the number of somatic cells to be used for nuclear reprogramming is not limited, but ranges from approximately 5×10′ to approximately 5×10⁶ cells per culture dish (100 cm²).

When a gene containing a drug resistance gene is used as a marker gene, cells expressing the marker gene can be selected by culturing the cells in a medium (i.e., a selective medium) containing the relevant drug. Also, cells expressing the marker gene can be detected when the marker gene is a fluorescent protein gene, through observation with a fluorescence microscope, by adding a luminescent substrate in the case of a luminescent enzyme gene, or adding a chromogenic substrate in the case of a chromogenic enzyme gene.

As used herein, the term “somatic cell” refers to any cell (including matured cell, somatic stem cell or tissue stem cell, and precursor cell) excluding germline cells and ES cells. Examples of “somatic cells” for induction of iPS cells, as used herein, include, but are not limited to, keratinizing epithelial cells (e.g., keratinizing epidermal cells), mucosal epithelial cells (e.g., epithelial cells of the surface layer of tongue), exocrine epithelial cells (e.g., mammary glandular cells), hormone-secreting cells (e.g., adrenal medullary cells), cells for metabolism and storage (e.g., hepatocytes), boundary-forming luminal epithelial cells (e.g., type I alveolar cells), luminal epithelial cells of internal tubules (e.g., vascular endothelial cells), ciliated cells having carrying capacity (e.g., airway epithelial cells), cells for secretion to extracellular matrix (e.g., fibroblasts), contractile cells (e.g., smooth muscle cells), cells of blood and immune system (e.g., T lymphocytes), cells involved in sensation (e.g., rod cells), autonomic nervous system neurons (e.g., cholinergic neurons), sense organ and peripheral neuron supporting cells (e.g., satellite cells), nerve cells and glial cells of the central nervous system (e.g., astroglial cells), chromocytes (e.g., retinal pigment epithelial cells), and precursor cells thereof (tissue precursor cells). Without particular limitation concerning the degree of cell differentiation, both undifferentiated precursor cells (also including somatic stem cells) and terminally-differentiated mature cells can be similarly used as origins for somatic cells in the present invention. Examples of undifferentiated precursor cells include tissue stem cells (somatic stem cells) such as neural stem cells, hematopoietic stem cells, mesenchymal stem cells, and dental pulp stem cells.

### <Method of Inducing Differentiation of Vascular Endothelial Cells>

An induction differentiation method that can be used for producing vascular endothelial cells from iPS cells obtained as described above comprises the following steps of:

(1) performing adhesion culture using a medium for primate ES/iPS cells on a coated culture dish;

(2) adding various additives to the medium and culturing cells;

(3) adding a growth factor to a serum free medium and culturing cells;

(4) separating VEGFR2-positive, TRA1-negative and VE-cadherin-positive cells; and

(5) performing adhesion culture using a growth medium for vascular endothelial cells on a coated culture dish.

Vascular endothelial cells in the present invention preferably express a vascular endothelial cell marker such as VE-cadherin, CD31, CD34, or eNOS and have a cobblestoned appearance.

Prior to the above step (1), iPS cells can be dissociated therefrom by an arbitrary method. For a method for dissociation, a mechanical dissociation or a dissociation solution having both protease activity and collagenase activity (e.g., Accutase (trademark) (Invitrogen) or Accumax (trademark) (Accumax)) or having collagenase activity alone can be used.

Also, examples of a coating agent to be used in steps (1) and (5) include Matrigel (BD), type-I collagen, type-IV collagen, gelatin, laminin, heparan sulfate proteoglycan, and entactin, and combinations thereof. A preferable example of the same in step (1) is type-I collagen. A preferable example of the same in step (5) is type-IV collagen.

A medium for producing vascular endothelial cells can be prepared using a medium used for culturing animal cells, as a basal medium. Examples of the basal medium include IMDM, Medium 199, Eagle's Minimum Essential Medium (EMEM), alpha MEM, Doulbecco's modified Eagle's Medium (DMEM), Ham's F12 medium, RPMI 1640 medium, Fischer's medium, and mixtures thereof. Furthermore, such a medium may contain serum or may be serum free. If necessary, for example, a medium may contain one or more serum substitutes, such as albumin, transferrin, Knockout Serum Replacement (KSR) (substitute for FBS upon culture of ES cells), fatty acids, insulin, a collagen progenitor, trace elements, 2-mercaptoethanol, 3′-thiolglycerol, as well as, one or more substances such as lipids, amino acids, L-glutamine, Glutamax (Invitrogen), nonessential amino acids, vitamins, antibiotics, antioxidants, pyruvic acid, buffering agents, inorganic salts, N2 supplement (Invitrogen), B27 supplement (Invitrogen), GSK-3 alpha/beta inhibitor, and growth factors such as VEGF. Examples of the medium that contains these additives in advance include a medium for primate ES/iPS cells (ReproCELL, Japan), Stem Pro (trademark) (Invitrogen), and a growth medium for vascular endothelial cells (Lonza). Examples of preferable media in the present invention include a medium for primate ES/iPS cells in step (1), a medium for primate ES/iPS cells supplemented with the N2 supplement, the B27 supplement, and the GSK-3 alpha/beta inhibitor in step (2), Stem Pro (trademark) containing VEGF in step (3), and a growth medium for vascular endothelial cells in step (5).

Examples of the GSK-3 alpha/beta inhibitor include SB216763, SB415286, FRAT1/FRAT2, Lithium, Kempaullone, Alsterpaullone, Indiubin-3′-oxime, BIO, TDZD-8, and Ro31-8220.

The temperature for culture ranges from about 30 degrees C. to 40 degrees C. and is preferably about 37 degrees C., but the examples are not limited thereto. Culture is carried out under atmosphere containing CO₂. CO₂ concentration preferably ranges from about 2 to 5%. The time for culture is not particularly limited and ranges from 1 to 2 days and more preferably 1 day in step (1), ranges from 2 to 5 days and more preferably 3 days in step (2), ranges from 3 to 7 days and more preferably 5 days in step (3), and ranges from 3 or more days in step (5), for example.

VEGFR2-postive, TRA1-negative, and VE-cadherin-positive cells can be separated by a method known by persons skilled in the art from cells stained with anti-VEGFR2, anti-TRA1, and anti-VE-cadherin antibodies using a flow cytometer or the like.

Here, in the case of vascular endothelial cells prepared as described above from iPS cells formed from subjects suffered from polycystic kidney disease and having cerebral aneurysm as a complication, the expression levels of NTNG1, POSTN, TNC, KAL1, and BST1 are lower and the expression levels of ACAT2, INSIG1, and SCD are higher than those in vascular endothelial cells prepared from iPS cells derived from a healthy subject not affected by polycystic kidney disease.

Moreover, in the case of vascular endothelial cells prepared as described above from iPS cells formed from subjects suffered from polycystic kidney disease and having cerebral aneurysm as a complication, the expression levels of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in vascular endothelial cells prepared from iPS cells formed from subjects suffered from polycystic kidney disease but not affected by cerebral aneurysm.

### <Method of Inducing Differentiation of Vascular Mural Cells>

For production of vascular mural cells, an induction differentiation method that can be used herein comprises the steps analogous to the above steps (1) to (3) for preparation of vascular endothelial cells, followed by steps (4′) and (5′):

(1) performing adhesion culture using a medium for primate ES/iPS cells on a coated culture dish;

(2) adding various additives to the medium and culturing cells;

(3) adding a growth factor to a serum free medium and culturing cells;

(4′) separating VEGFR2-positive, TRA1-negative, and VE-cadherin-negative cells; and

(5′) performing adhesion culture using a medium containing a growth factor on a coated culture dish.

In the present invention the vascular mural cells are involved of pericytes or smooth muscle cells. Vascular mural cells in the present invention are preferably spindle-shaped cells expressing vascular mural cell markers such as alpha smooth muscle actin and calponin.

A medium used in step (5′) can be prepared using a medium used for culturing animal cells as a basal medium. Examples of the basal medium include IMDM, Medium 199, Eagle's Minimum Essential Medium (EMEM or MEM), alpha MEM, Doulbecco's modified Eagle's Medium (DMEM), Ham's F12 medium, an RPMI 1640 medium, Fischer's medium, and mixtures thereof. Furthermore, such a medium may contain serum or may be serum free. If necessary, for example, a medium may contain one or more serum substitutes, such as albumin, transferrin, Knockout Serum Replacement (KSR) (i.e., a substitute for FBS upon culture of ES cells), fatty acids, insulin, a collagen progenitor, trace elements, 2-mercaptoethanol, 3′-thiolglycerol, as well as, one or more substances such as lipids, amino acids, L-glutamine, Glutamax (Invitrogen), nonessential amino acids, vitamins, antibiotics, antioxidants, pyruvic acid, buffering agents, inorganic salts, N2 supplement (Invitrogen), B27 supplement (Invitrogen), a GSK-3 alpha/beta inhibitor, and growth factors such as PDGF-BB. An example of preferable medium is MEM containing 2-% FCS and PDGF-BB.

The temperature for culture ranges from about 30 degrees C. to 40 degrees C. and is preferably about 37 degrees C., but the examples are not limited thereto. Culture is carried out under atmosphere containing CO₂. CO₂ concentration preferably ranges from about 2 to 5%. The time for culture is not particularly limited and is 3 days or more in the step (5′), for example.

VEGFR2-positive, TRA1-negative, and VE-cadherin-negative cells can be separated by a method known by persons skilled in the art from cells stained with anti-VEGFR2, anti-TRA1, and anti-VE-cadherin antibodies using a flow cytometer or the like.

Here, in the case of vascular mural cells produced as described above from iPS cells formed from somatic cells of subjects suffered from polycystic kidney disease and having cerebral aneurysm as a complication, the expression levels of HSD3B 1, KRT7, USP40, and SULT1E1 are lower than those in iPS cells formed from a healthy subject not affected by polycystic kidney disease.

Moreover, in the case of vascular mural cells prepared as described above from iPS cells formed from somatic cells of subjects suffered from polycystic kidney disease and having cerebral aneurysm as a complication, the expression levels of MMP1, TFPI2, HMGA2, KRTAP4-7, KRTAP4-8, KRTAP4-9, MYPN, RPPH1, and SIAE are higher than those in vascular mural cells prepared from iPS cells formed from somatic cells of subjects suffered from polycystic kidney diseasebut not affected by cerebral aneurysm.

### <Screening Method>

The present invention provides a method of screening for a candidate drug that is useful for treating or preventing polycystic kidney disease or a complication of polycystic kidney disease. Through the use of such a screening method using the expression levels of the genes listed in Table 1 as indicators, the therapeutic agent(s) or the preventive agent(s) can be identified. The expression levels of the genes listed in Table 1 (above) can be measured using the above disease markers.

In the present invention, the method of screening for a therapeutic agent or a preventive agent for polycystic kidney disease can comprise the following steps of:

(A-1) bringing each of candidate substances into contact with a somatic cell obtained by differentiation induction from iPS cells formed from a somatic cell of a subject suffered from polycystic kidney disease;

(B-1) measuring the expression levels or the transcriptional activity of 1 or more genes selected from or all genes of a group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and

(C-1) selecting a candidate substance as a therapeutic agent or a preventive agent for polycystic kidney disease when the expression levels or transcriptional activity of 1 or more genes selected from or all genes of a group consisting of NTNG1, POSTN, TNC, KAL1, BST1, HSD3B1, KRT7, USP40, and SULT1E1 exhibit an increase compared with a case in which the candidate substance is not brought into contact, or, selecting a candidate substance as a therapeutic agent or a preventive agent for polycystic kidney disease when the expression levels or transcriptional activity of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE exhibit a decrease compared with a case in which the candidate substance is not brought into contact. Here, examples of somatic cells obtained via differentiation induction from iPS cells include tubular cells, collecting tubule cells, bile duct cells, hepatocytes, pancreatic ductal cells, pancreatic cells, bowel cells, germ cells, vascular endothelial cells, and vascular mural cells. Preferable examples thereof include vascular endothelial cells and vascular mural cells. Methods for producing tubular cells, collecting tubule cells, bile duct cells, hepatocytes, pancreatic ductal cells, pancreatic cells, bowel cells, germ cells, vascular endothelial cells, or vascular mural cells from iPS cells are not particularly limited. Somatic cells as used herein can be obtained by appropriately extracting from embryoid bodies or formed teratomas (e.g., JP Patent Publication (Kokai) No. 2006-239169 A). Hepatocytes are not particularly limited and can be prepared by the method according to WO2006/082890, JP Patent Publication (Kokai) No. 2010-75631 A or Hay D C, et al, Proc Natl Acad Sci U.S.A., 105, 12301-6, 2008. Similarly, pancreatic cells can be prepared using the method according to WO2007/103282. iPS cells and vascular endothelial cells or vascular mural cells can be produced by the above methods.

Preferably, the method of screening for a therapeutic agent or a preventive agent for polycystic kidney disease uses vascular endothelial cells and can comprise the following steps of:

(A-2) bringing each of candidate substances into contact with a vascular endothelial cell obtained via differentiation induction from iPS cells formed from a subject suffered from polycystic kidney disease;

(B-2) measuring the expression levels or the transcriptional activity of a gene(s) selected from among NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2; and

(C-2) selecting a candidate substance as a therapeutic agent or a preventive agent for polycystic kidney disease when the expression levels or the transcriptional activity of 1 or more genes selected from or all genes of a group consisting of NTNG1, POSTN, TNC, KAL1, and BST1 exhibit an increase compared with a case in which the candidate substance is not brought into contact, or, selecting a candidate substance as a therapeutic agent or a preventive agent for polycystic kidney disease when the expression levels or the transcriptional activity of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibits a decrease compared with a case in which the candidate substance is not brought into contact.

Alternatively, the method using vascular mural cells can comprise the following steps of:

(A-3) bringing each of candidate substances into contact with vascular mural cells obtained via differentiation induction from iPS cells formed from a somatic cell of a subject suffered from polycystic kidney disease;

(B-3) measuring the expression levels or the transcriptional activity of 1 or more genes selected from or all genes of a group consisting of HSD3B1, KRT7, USP40, SULT1E1, MMP1, TFPI2, HMGA2, KRTAP4-7, KRTAP4-8, KRTAP4-9, MYPN, RPPH1, and SIAE; and

(C-3) selecting a candidate substance as a therapeutic agent or a preventive agent for polycystic kidney disease when the expression levels or the transcriptional activity of 1 or more genes selected from or all genes of a group consisting of HSD3B1, KRT7, USP40, and SULT1E1 exhibit an increase compared with a case in which the candidate substance is not brought into contact, or, selecting a candidate substance as a therapeutic agent or a preventive agent for polycystic kidney disease when the expression levels or the transcriptional activity of 1 or more genes selected from or all genes of a group consisting of MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

In another aspect, the method of screening for a therapeutic agent or a preventive agent for a complication of polycystic kidney disease can comprise the following steps of:

(A-4) bringing each of candidate substances into contact with a somatic cell obtained via differentiation induction from iPS cells formed from a somatic cell of a patient with a complication of polycystic kidney disease;

(B-4) measuring the expression levels or the transcriptional activity of 1 or more genes selected from or all genes of a group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and

(C-4) selecting a candidate substance as a therapeutic agent or a preventive agent for a complication of polycystic kidney disease when the expression levels or the transcriptional activity of 1 or more genes selected from or all genes of a group consisting of NTNG1, POSTN, TNC, KAL1, BST1, HSD3B1, KRT7, USP40, and SULT1E1 exhibit an increase compared with a case in which the candidate substance is not brought into contact, or, selecting a candidate substance as a therapeutic agent or a preventive agent for a complication of polycystic kidney disease when the expression levels or the transcriptional activity of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

In the present invention, examples of a complication of polycystic kidney disease include vascular lesions such as aortic aneurysm, cerebral aneurysm, or subarachnoid hemorrhage, valvular disease of heart, diverticula of colon, hernia, ductal lesions such as choledochal dilatation, as well as cyst formation in the liver, the pancreas, the spleen and generative organs. An example of a complication to be particularly detected is cerebral aneurysm.

Preferably, the method of screening for a therapeutic agent or a preventive agent for a complication of polycystic kidney disease uses vascular endothelial cells and comprises the following steps of:

(A-5) bringing each of candidate substances into contact with a vascular endothelial cell obtained via differentiation induction from iPS cells formed from a somatic cell of a patient with a complication of polycystic kidney disease;

(B-5) measuring the expression levels or the transcriptional activity of 1 or more genes selected from or all genes of a group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2; and

(C-5) selecting a candidate substance as a therapeutic agent or a preventive agent for a complication that accompanies polycystic kidney disease when the expression levels or the transcriptional activity of 1 or more genes selected from or all genes of a group consisting of NTNG1, POSTN, TNC, KAL1, and BST1 exhibit an increase compared with a case in which the candidate substance is not brought into contact, or, selecting a candidate substance as a therapeutic agent or a preventive agent for a complication that accompanies polycystic kidney disease when the expression levels or the transcriptional activity of 1 or more genes selected from or all genes of a group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibit a decrease compared with a case in which the candidate substance is not brought into contact.

More preferably, the method of screening for a therapeutic agent or a preventive agent for cerebral aneurysm that accompanies polycystic kidney disease uses vascular endothelial cells and comprises the following steps of:

(A-6) bringing candidate substances into contact with vascular endothelial cells obtained via differentiation induction from iPS cells formed from a patient with a complication of polycystic kidney disease,

(B-6) measuring the expression levels or the transcriptional activity of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2, and

(C-6) selecting a candidate substance as a therapeutic agent or a preventive agent for cerebral aneurysm that accompanies polycystic kidney disease when the expression levels or the transcriptional activity of 1 or more genes selected from or all genes of a group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibits a decrease compared with a case in which the candidate substance is not brought into contact.

Alternatively, the relevant method uses vascular mural cells and can comprise the following steps of:

(A-7) bringing each of candidate substances into contact with vascular mural cells obtained via differentiation induction from iPS cells formed from a somatic cell of a subject with a complication of polycystic kidney disease;

(B-7) measuring the expression levels or the transcriptional activity of 1 or more genes selected from or all genes of a group consisting of HSD3B1, KRT7, USP40, SULT1E1, MMP1, TFPI2, HMGA2, KRTAP4-7, KRTAP4-8, KRTAP4-9, MYPN, RPPH1, and SIAE; and

(C-7) selecting a candidate substance as a therapeutic agent or a preventive agent for a complication that accompanies polycystic kidney disease when the expression levels or the transcriptional activity of 1 or more genes selected from or all genes of a group consisting of HSD3B1, KRT7, USP40, and SULT1E1 exhibit an increase compared with a case in which the candidate substance is not brought into contact, or, selecting a candidate substance as a therapeutic agent or a preventive agent for a complication that accompanies polycystic kidney disease when the expression levels or the transcriptional activity of 1 or more genes selected from or all genes of a group consisting of MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

Preferably, the method of screening for a therapeutic agent or a preventive agent for cerebral aneurysm that accompanies polycystic kidney disease uses vascular mural cells and can comprise the following steps of:

(A-8) bringing each of candidate substances into contact with vascular mural cells obtained via differentiation induction of iPS cells formed from a somatic cell of a subject with a complication of polycystic kidney disease;

(B-8) measuring the expression levels or the transcriptional activity of 1 or more genes selected from or all genes of a group consisting of MMP1, TFPI2, HMGA2, KRTAP4-7, KRTAP4-8, KRTAP4-9, MYPN, RPPH1, and SIAE; and

(C-8) selecting a candidate substance as a therapeutic agent or a preventive agent for a complication that accompanies polycystic kidney disease when the expression levels or the transcriptional activity of 1 or more genes selected from or all genes of a group consisting of MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

Here, the above expression levels can be detected using the above disease markers. The above transcriptional activity can be detected using a reporter gene that is controlled by the transcriptional regulatory region thereof. Specifically, the method can comprise the following steps of:

(A-9) bringing each of candidate substances into contact with a somatic cell obtained via differentiation induction from iPS cells formed from a somatic cell of a subject suffered from polycystic kidney disease, wherein the somatic cell contain a reporter gene that is controlled by the transcriptional regulatory region of 1 or a plurality of genes selected from among NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE;

(B-9) measuring the expression or the activity of the reporter gene; and

(C-9) selecting a candidate substance that increases the expression level or the activity level of the reporter gene compared with the expression level in the absence of the candidate substance, when the selected gene(s) is NTNG1, POSTN, TNC, KAL1, BST1, HSD3B1, KRT7, USP40 and/or SULT1E1, or, selecting a candidate substance that decreases the expression level of the reporter gene when the selected gene(s) is ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1 and/or SIAE compared with the expression level detected in the absence of the candidate substance.

Preferably, the relevant method uses vascular endothelial cells as somatic cells obtained via differentiation induction from iPS cells and can comprise the following steps of:

(A-10) bringing each of candidate substances into contact with a vascular endothelial cell obtained via differentiation induction from iPS cells formed from a somatic cell of a subject suffered from polycystic kidney disease, wherein the vascular endothelial cell contains a reporter gene that is controlled by the transcriptional regulatory region of 1 or a plurality of genes selected from among NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2;

(B-10) measuring the expression or the activity of the reporter gene; and

(C-10) selecting a candidate substance that increases the expression level or the activity level of the reporter gene compared with the expression level in the absence of the candidate substance when the selected gene(s) is NTNG1, POSTN, TNC, KAL1 and/or BST1, or selecting a candidate substance that decreases the expression level of the reporter gene compared with the expression level detected in the absence of the candidate substance, when the selected gene(s) is ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1 and/or TFPI2. Alternatively, the method uses vascular mural cells and can comprise the following steps of:

(A-11) bringing each of candidate substances into contact with vascular mural cells obtained via differentiation induction from iPS cells of a subject suffered from polycystic kidney disease, wherein the vascular mural cells contain a reporter gene that is controlled by the transcriptional regulatory region of 1 or a plurality of genes selected from among HSD3B1, KRT7, USP40, SULT1E1, MMP1, TFPI2, HMGA2, KRTAP4-7, KRTAP4-8, KRTAP4-9, MYPN, RPPH1 and/or SIAE;

(B-11) measuring the expression or the activity of the reporter gene; and

(C-11) selecting a candidate substance that increases the expression level or the activity level of the reporter gene compared with the expression level detected in the absence of the candidate substance when the selected gene(s) is HSD3B 1, KRT7, USP40 and/or SULT1E1, or selecting a candidate substance that decreases the expression level of the reporter gene compared with the expression level detected in the absence of the candidate substance when the gene is MMP1, TFPI2, HMGA2, KRTAP4-7, KRTAP4-8, KRTAP4-9, MYPN, RPPH1 and/or SIAE.

In another aspect, the method of screening for a therapeutic agent or a preventive agent for a complication of polycystic kidney disease can comprise the following steps of:

(A-12) bringing each of candidate substances into contact with somatic cells obtained via differentiation induction from iPS cells formed from a somatic cell of asubject with a complication of polycystic kidney disease, wherein the somatic cells contain a reporter gene that is controlled by the transcriptional regulatory region of 1 or a plurality of genes selected from among NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE;

(B-12) measuring the expression or the activity of the reporter gene; and

(C-12) selecting a candidate substance that increases the expression level or the activity level of the reporter gene compared with the expression level in the absence of the candidate substance when the selected gene(s) is NTNG1, POSTN, TNC, KAL1, BST1, HSD3B1, KRT7, USP40 and/or SULT1E1, or selecting a candidate substance that decreases the expression level of the reporter gene compared with the expression level detected in the absence of the candidate substance when the selected gene(s) is ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1 and/or SIAE.

Preferably, the method uses vascular endothelial cells as somatic cells obtained via differentiation induction from iPS cells and can comprise the following steps of:

(A-13) bringing each of candidate substances into contact with vascular endothelial cells obtained via differentiation induction from iPS cells formed from a somatic cell of a subject with a complication of polycystic kidney disease, wherein the endothelial cells contain a reporter gene that is controlled by the transcriptional regulatory region of 1 or a plurality of genes selected from among NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2;

(B-13) measuring the expression or the activity of the reporter gene; and

(C-13) selecting a candidate substance that increases the expression level or the activity level of the reporter gene compared with the expression level in the absence of the candidate substance when the selected gene(s) is NTNG1, POSTN, TNC, KAL1 and/or BST1, or selecting a candidate substance that decreases the expression level of the reporter gene compared with the expression level detected in the absence of the candidate substance when the selected gene(s) is ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1 and/or TFPI2. More preferably, the method uses vascular endothelial cells as somatic cells obtained via differentiation induction from iPS cells and comprises the following steps of:

(A-14) bringing each of candidate substances into contact with a vascular endothelial cell obtained via differentiation induction from iPS cells formed from a somatic cell of a subject suffered from polycystic kidney disease who also develops cerebral aneurysm as a complication, wherein the endothelial cells contain a reporter gene that is controlled by the transcriptional regulatory region of 1 or a plurality of genes selected from among BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2;

(B-14) measuring the expression or the activity of the reporter gene; and

(C-14) selecting a candidate substance that decreases the expression level of the reporter gene compared with the expression level detected in the absence of the candidate substance when the selected gene(s) is BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and/or TFPI2.

Alternatively, the method uses vascular mural cells and can comprise the following steps of:

(A-15) bringing each of candidate substances into contact with vascular mural cells obtained via differentiation induction from iPS cells formed from a somatic cell of a subject with a complication of polycystic kidney disease, wherein the vascular mural cells contain a reporter gene that is controlled by the transcriptional regulatory region of 1 or a plurality of genes selected from among HSD3B1, KRT7, USP40, SULT1E1, MMP1, TFPI2, HMGA2, KRTAP4-7, KRTAP4-8, KRTAP4-9, MYPN, RPPH1, and SIAE;

(B-15) measuring the expression or the activity of the reporter gene; and

(C-15) selecting a candidate substance that increases the expression level or the activity level of the reporter gene compared with the expression level in the absence of the candidate substance when the selected gene(s) is HSD3B1, KRT7, USP40 and/or SULT1E1, or selecting a candidate substance that decreases the expression level of the reporter gene compared with the expression level detected in the absence of the candidate substance when the selected gene(s) is MMP1, TFPI2, HMGA2, KRTAP4-7, KRTAP4-8, KRTAP4-9, MYPN, RPPH1 and/or SIAE.

Preferably, the method uses vascular mural cells as somatic cells obtained via differentiation induction from iPS cells and can comprise the following steps of:

(A-16) bringing each of candidate substances into contact with vascular mural cells obtained via differentiation induction from iPS cells formed from a somatic cell of a subject suffered from polycystic kidney disease who also develops cerebral aneurysm as a complication, wherein the vascular mural cells contain a reporter gene that is controlled by the transcriptional regulatory region of 1 or a plurality of genes selected from among MMP1, TFPI2, HMGA2, KRTAP4-7, KRTAP4-8, KRTAP4-9, MYPN, RPPH1, and SIAE;

(B-16) measuring the expression or the activity of the reporter gene; and

(C-16) selecting a candidate substance that decreases the expression level of the reporter gene compared with the expression level detected in the absence of the candidate substance when the selected gene(s) is MMP1, TFPI2, HMGA2, KRTAP4-7, KRTAP4-8, KRTAP4-9, MYPN, RPPH1 and/or SIAE.

In the present invention, the transcriptional regulatory regions of the genes listed in Table 1 (see above) can be isolated from the genomic libraries based on the nucleotide sequence information of the genes. Cells containing a reporter gene that is controlled by the transcriptional regulatory region of the gene can be prepared by introducing a vector containing the reporter gene sequence operably linked to the transcriptional regulatory region sequence into cells. In another aspect, with the use of the homologous recombination method, a reporter gene sequence may be inserted downstream of the transcriptional regulatory region by a method known by persons skilled in the art, so that it is operably linked thereto.

The above vector introduction and homologous recombination method may be performed in any of cells including somatic cells, iPS cells, vascular endothelial cells, and vascular mural cells. The homologous recombination method is desirably performed using iPS cells.

In the present invention, reporter genes known in the art can be used as appropriate reporter genes, and include, but are not particularly limited, green fluorescent protein (GFP), yellow fluorescent protein (YFP), red fluorescent protein (RFP), luciferase, beta glucuronidase (GUS), beta-galactosidase, HRP, chloramphenicol acetyltransferase, or the like.

In the screening method of the present invention, arbitrary candidate substances can be used. Examples of candidate substances include, but are not limited to, cell extracts, cell culture supernatants, microbial fermentation products, marine organism-derived extracts, plant extracts, purified proteins or crude proteins, peptides, non-peptide compounds, synthetic low-molecular-weight compounds, and natural compounds. In the present invention, candidate substances can also be obtained using any one of many approaches in combinatorial library methods known in the art including (1) a biological library method, (2) a synthetic library method using deconvolution, (3) a “onebead one-compound” library method, and (4) a synthetic library method using affinity chromatography selection. An example of the biological library method using affinity chromatography selection is limited to a peptide library method, however, the other 4 approaches can be applied to peptides, non-peptide oligomers, or low-molecular-weight compound libraries (Lam (1997) Anticancer Drug Des. 12: 145-67). Examples of a method for synthesizing a molecular library can be found in the art (DeWitt et al. (1993) Proc. Natl. Acad. Sci. U.S.A. 90: 6909-13; Erb et al. (1994) Proc. Natl. Acad. Sci. U.S.A. 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33: 2059; Carell et al. (1994) Angew. Chem. Int. Ed. Engl. 33: 2061; Gallop et al. (1994) J. Med. Chem. 37: 1233-51). Compound libraries can be constructed in the form of solutions (see Houghten (1992) Bio/Techniques 13: 412-21) or beads (Lam (1991) Nature 354: 82-4), chips (Fodor (1993) Nature 364: 555-6), bacteria (U.S. Pat. No. 5,223,409), spores (U.S. Pat. No. 5,571,698, U.S. Pat. No. 5,403,484, and U.S. Pat. No. 5,223,409), plasmids (Cull et al. (1992) Proc. Natl. Acad. Sci. U.S.A. 89: 1865-9), or phages (Scott and Smith (1990) Science 249: 386-90; Devlin (1990) Science 249: 404-6; Cwirla et al. (1990) Proc. Natl. Acad. Sci. U.S.A. 87: 6378-82; Felici (1991) J. Mol. Biol. 222: 301-10; U.S. Patent Application No. 2002103360).

<Antisense Oligonucleotide, or siRNA or shRNA, and Therapeutic Agent Produced Using them>

An example of an antisense oligonucleotide in the present invention is an oligonucleotide hybridizing to a site within the nucleotide sequence of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2 HMGA2, KRTAP4-7, KRTAP4-8, KRTAP4-9, MYPN, RPPH1, or SIAE. Such an antisense oligonucleotide has a nucleotide sequence complementary to the nucleotide sequence comprising at least 15 continuous nucleotides (e.g., 20 to 200 continuous nucleotides) in the nucleotide sequence of preferably SEQ ID NO: 11, 13, 45, 47, 49, 51, 53, 55, 57, 59, 61, 63, 65, 67, 69, 71, 73, 75, 77, 79, 81, 83, 85, 87, 89, 91, 93, or 94. A further preferable antisense oligonucleotide contains an initiation codon in the above at least 15 continuous nucleotides.

A derivative or a modification product of the above antisense oligonucleotide can be used as an antisense oligonucleotide. Examples of such modification include a methylphosphonate-type or an ethyl-phosphonate-type lower alkyl phosphonate modification, phosphorothioate modification, and phosphoramidite modification.

In the present invention, the nucleotide sequence of siRNA or shRNA can be designed using an siRNA design computer program that is available from www.ambion.com/techlib/misc/siRNA_finder.html. The computer program is used for selecting a nucleotide sequence for siRNA or shRNA synthesis based on the following protocols.

An siRNA or shRNA target site can be selected as follows.

(1) A downstream portion from the AUG initiation codon as a starting point of ACAT2 (SEQ ID NO: 45), INSIG1 (SEQ ID NO: 11), SCD (SEQ ID NO: 13), BMP6 (SEQ ID NO: 47), CD274 (SEQ ID NO: 49), CTGF (SEQ ID NO: 51), E2F7 (SEQ ID NO: 53), EDN1(SEQ ID NO: 55), FAM43A (SEQ ID NO: 57), FRMD3 (SEQ ID NO: δ 59), MMP10 (SEQ ID NO: 61), MYEOV (SEQ ID NO: 63), NR2F1(SEQ ID NO: 65), NRCAM(SEQ ID NO: 67), PCSK1 (SEQ ID NO: 69), PLXNA2 (SEQ ID NO: 71), SLC30A3 (SEQ ID NO: 73), SNAI1 (SEQ ID NO: 75), SPOCD1 (SEQ ID NO: 77), MMP1 (SEQ ID NO: 79), TFPI2 (SEQ ID NO: 81), HMGA2 (SEQ ID NO: 83), KRTAP4-7 (SEQ ID NO: 85), KRTAP4-8 (SEQ ID NO: 87), KRTAP4-9 (SEQ ID NO: 89), MYPN (SEQ ID NO: 91), RPPH1 (SEQ ID NO: 93), or SIAE (SEQ ID NO: 94) is scanned for an AA dinucleotide sequence. As a useful siRNA or shRNA target site, 19 nucleotides adjacent to the 3′ side, or a site in which each AA appears, is used as a candidate sequence. According to Tuschl, et al. (1999) Genes Dev 13: 3191-7, the design of siRNA or shRNA for the 5′-untranslated region (UTR) and the 3′-UTR and a region (within 75 nucleotides) near the initiation codon is not recommended since an UTR binding protein and/or a translation initiation complex interferes with the binding of an siRNA endonuclease complex.

(2) A target site and a human genome data base are compared, so as to exclude all target sequences having significant sequence identity with other coding sequences. Sequence identity can be examined using BLAST (Altschul S F, et al, (1997) Nucleic Acids Res. 25 (17): 3389-402 or (1990) J Mol. Biol. 215 (3): 403-10) found on the NCBI server, ncbi.nlm.nih.gov/BLAST/.

(3) A target sequence appropriate for synthesis is selected.

A sense strand comprising the thus selected siRNA target site may form double stranded RNA through hybridization with a corresponding antisense strand. In another embodiment, single-stranded RNA in which the sense strand and the antisense strand corresponding thereto are linked via a loop sequence, i.e. shRNA, can form a hair-pin loop structure. Such double stranded RNA may contain mismatch of 1 (or less) nucleotide per 10 nucleotides. Preferably, strands of a double stranded complex are completely complementary to each other, containing no mismatch. The present invention also encompasses a vector containing 1 or a plurality of nucleic acids of the above sense strand and cells containing the vector.

As an siRNA composition, a vector comprising a sequence (shRNA) ligated to comprise a hair-pin loop structure comprising a sense strand, an antisense strand, or both strands, so as to be transcribed by an RNA polymerase III transcriptional unit (e.g., an intranuclear low molecular weight RNA (snRNA) U6 promoter, or a human H1 RNA promoter) can be used. Alternatively, double stranded RNA may be directly used.

Double stranded RNA may be chemically stabilized in order to prevent in vivo degradation by nuclease. A method of preparing chemically stabilized RNA molecules is known in the art. Typically, such a molecule contains a backbone modified to be able to avoid the action of ribonuclease and nucleotides. Another example of modification is cholesterol conjugated siRNA (Song et al, (2003) Nature Med. 9: 347-51). An siRNA composition or an shRNA composition may further contain liposomes (e.g., cationic liposomes or anionic liposomes), nanoparticles, or a viral vector including retrovirus, adenovirus, or adeno-associated virus. Moreover, an siRNA composition or an shRNA composition may contain a pharmaceutically acceptable carrier such as physiological saline.

The above siRNA composition or shRNA composition can be used for treatment of polycystic kidney disease or a complication of polycystic kidney disease through parenteral administration such as, an intravenous, subcutaneous, intramuscular, or intraperitoneal administration route. The above siRNA composition or shRNA composition may be directly injected to an affected portion.

The dose of an antisense oligonucleotide, siRNA, or shRNA depends on many factors including body weight, age, gender, administration time, and the route of administration, symptoms, and other medicaments to be administered simultaneously. The dose of the above drug for intravenous administration desirably ranges from about 10⁶ to 10²² copies.

### EXAMPLES

The present invention will hereafter be described in more detail with reference to the following examples, although the technical scope of the present invention is not limited thereto.

### Example 1

### Establishment of iPS Cell Lines

### <Fibroblast Cells>

Fibroblasts were established by culturing skin samples obtained via biopsies from four autosomal dominant polycystic kidney disease patients with onset of cerebral aneurysm as a complication and from three autosomal dominant polycystic kidney disease patients not developing cerebral aneurysm under agreement. The resultants were each designated PK-ane fibroblasts and PK-free fibroblasts, respectively, and then used in this Example. Meanwhile, a fibroblast cell line of three healthy persons not developing autosomal dominant polycystic kidney disease and cerebral aneurysm is designated nonPK fibroblast and then used in this Example.

### <Ips Cell Induction>

Human cDNAs corresponding to Oct3/4, Sox2, Klf4, and c-Myc were introduced into the above fibroblasts using retrovirus according to the method described by Takahashi, K. et al. (Cell, 131(5), 861, 2007). Similarly, human cDNAs corresponding to Oct3/4, Sox2, and Klf4 were introduced into the above fibroblasts using a retrovirus according to the method as described by Nakagawa, M. et al. (Nat Biotechnol 26 (1), 101, 2008). After gene introduction, fibroblasts were transferred onto SNL feeder cells, followed by, on the next day, medium exchange with culture solutions (for primate ES cells) supplemented with 4 ng/ml bFGF (Wako). Colonies that had appeared were picked up, so that one iPS cell line was selected per fibroblast cell line. The thus selected PK-ane fibroblast-derived iPS cell lines were designated PK-ane-iPS 1, PK-ane-iPS2, PK-ane-iPS3, and PK-ane-iPS4; PK-free fibroblast-derived iPS cell lines were designated PK-free-iPS1, PK-free-iPS2, and PK-free-iPS3; as well as nonPK fibroblast-derived iPS cell lines were designated nonPK-iPS1, nonPK-iPS2, and nonPK-iPS3. Here, PK-ane-iPS1, PK-ane-iPS2, PK-ane-iPS4, PK-free-iPS2, PK-free-iPS3, nonPK-iPS2, and nonPK-iPS3 were prepared by introduction of four factors (Oct3/4, Sox2, Klf4, and c-Myc). PK-ane-iPS3, PK-free-iPS1, and nonPK-iPS1 were prepared by introduction of three factors (Oct3/4, Sox2, and Klf4).

### Example 2

### Induction of Differentiation into Vascular Endothelial Cells

Each iPS cell line colony prepared as described in Example 1 was separated into pieces with an appropriate size, sprayed over a type-I collagen coating dish (Becton Dickinson), followed by 1 day of culture in a medium for primate ES/iPS cells (ReproCELL) to adhere the cells to the dish surface. On day 2, a GSK-3 alpha/beta inhibitor (Sigma), N2 supplement, and B27 supplement (both, Invitrogen) were added and then cells were cultured for further 3 days. Then the medium was exchanged with a serum free medium for human hematopoietic stem cells (Invitrogen), and then 50 ng/ml VEGF (Peprotec Inc) was added. After further 5 days of culture, cells were dissociated, and then VEGFR2-positive, TRA1-negative, and VE-cadherin-positive cells were separated by FACS. Subsequently, the separated cells were sprayed over type-IV collagen coating dishes (Becton Dickinson), and then cultured in a growth medium for vascular endothelial cells (Lonza). At the stage where vascular endothelial cell markers such as VE-cadherin, CD31, CD34, and eNOS were expressed and vascular endothelial cell sheets having a cobblestoned appearance were constructed, cells were collected as vascular endothelial cells (EC). EC cells prepared from iPS cell lines were designated PK-ane-iPS1-EC, PK-ane-iPS2-EC, PK-ane-iPS3-EC, PK-ane-iPS4-EC, PK-free-iPS1-EC, PK-free-iPS2-EC, PK-free-iPS3-EC, nonPK-iPS1-EC, nonPK-iPS2-EC, and nonPK-iPS3-EC, respectively.

### Example 3

### Induction of Differentiation into Vascular Mural Cells

Each of the above prepared iPS cell line colonies was separated into pieces with an appropriate size, sprayed over a type-I collagen coating dish (Becton Dickinson), followed by 1 day of culture with a medium for primate ES/iPS cells (ReproCELL) to adhere the cells to the dish surface. On day 2, a GSK-3 alpha/beta inhibitor (Sigma), N2 supplement, and B27 supplement (both, Invitrogen) were added, and then cells were cultured for further 3 days. Then the medium was exchanged with a serum free medium for human hematopoietic stem cells (Invitrogen). After 5 days of culture, cells were dissociated, and then VEGFR2-positive, TRA1-negative, and VE-cadherin-negative cells were separated by FACS. Subsequently, the thus separated cells were sprayed over a type-IV collagen coating dish (Becton Dickinson) and further cultured in MEM containing 2-% FCS and 20 ng/ml PDGF-BB (Peprotech Inc). Thus, cells were induced to differentiate into vascular mural cells (MC) expressing vascular mural cell markers such as alpha smooth muscle actin and calponin and presenting spindle shapes and then collected. MC cells prepared from iPS cell lines were designated PK-ane-iPS1-MC, PK-ane-iPS2-MC, PK-ane-iPS3-MC, PK-ane-iPS4— MC, PK-free-iPS1-MC, PK-free-iPS2-MC, PK-free-iPS3-MC, nonPK-iPS1-MC, nonPK-iPS2-MC, and nonPK-iPS3-MC.

### Example 4

### Confirmation of Each Gene Expression

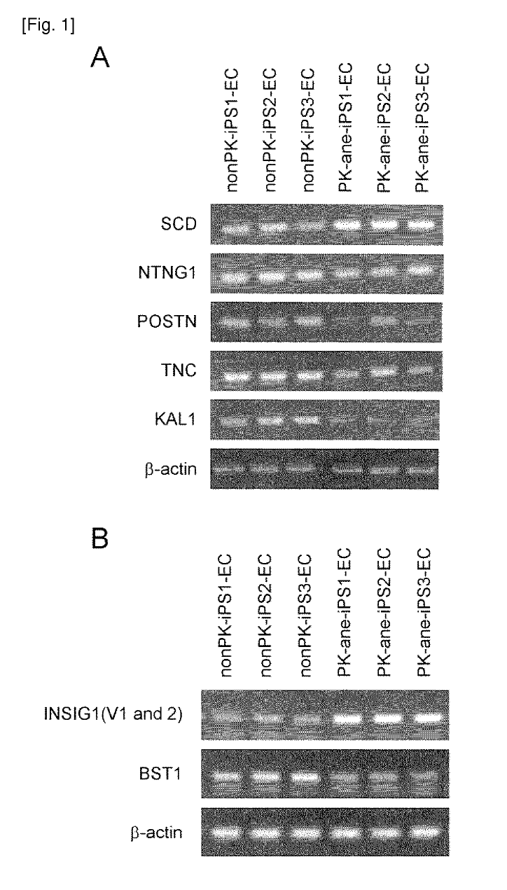

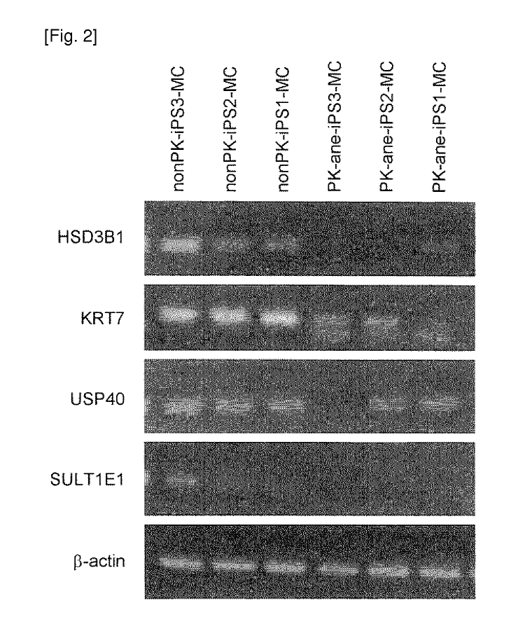

The expression levels of NTNG1, POSTN, TNC, KAL1, BST1, INSIG1, SCD, and ACAT2 in PK-ane-iPS1-EC, PK-ane-iPS2-EC, PK-ane-iPS3-EC, nonPK-iPS1-EC, nonPK-iPS2-EC, and nonPK-iPS3-EC obtained in Example 2 were measured by RT-PCR using primers listed in Table 2. The expression level of each gene in PK-iPS cell-derived EC was compared with that in nonPK-iPS cell-derived EC. Thus, it was confirmed that PK-ane-iPS cell-derived EC expressed NTNG1, POSTN, TNC, KAL1, and BST1 at low levels but expressed INSIG1, SCD, and ACAT2 at high levels (FIG. 1). Similarly, the expression levels of HSD3B1, KRT7, USP40, and SULT1E1 in PK-ane-iPS1-MC, PK-ane-iPS2-MC, PK-ane-iPS3-MC, nonPK-iPS1-MC, nonPKiPS2-MC, and nonPK-iPS3-MC obtained in Example 3 were measured by RT-PCR using primers listed in Table 2. The expression level of each gene in PK-iPS cell-derived MC was compared with that in nonPK-iPS cell-derived MC. It was confirmed that PK-iPS cell-derived MC expressed HSD3B 1, KRT7, USP40, and SULT1E1 at low levels (FIG. 2).

Subsequently, RNA extracted from PK-ane-iPS1-EC, PK-ane-iPS2-EC, PK-ane-iPS3-EC, PK-ane-iPS4-EC, PK-free-iPS1-EC, PK-free-iPS2-EC, and PK-free-iPS3-EC obtained in Example 2 was subjected to measurement of gene expression intensity using a microarray (Affymetrix). Groups with cerebral aneurysm as a complication and groups without cerebral aneurysm as a complication were compared for all 12 combinations of clones. Gene groups expressed at high levels in all groups with cerebral aneurysm as a complication are shown in Table 3. Also, the ratio of the expression level in PK-ane-iPS1-EC to that in PK-free-iPS1-EC in one case out of the above 12 combinations is shown in Table 3.

TABLE 3
Gene group expressed at high levels in vascular endothelial cells
obtained via differentiation induction from iPS cells formed
from fibroblast cells of PKD subjects with cerebral aneurysm

| Gene | Fold Change |
|---|---|
| Name | (PK-ane-iPS1-EC/PK-free-iPS1-EC) |
| SPOCD1 | 2.4045105 |
| PLXNA2 | 2.0494196 |
| MYEOV | 2.5981545 |
| MMP10 | 1.7859154 |
| MMP1 | 8.141003 |
| E2F7 | 3.2456584 |
| SLC30A3 | 2.6372058 |
| SNAI1 | 2.5232387 |
| FAM43A | 1.5843235 |
| NR2F1 | 1.7354009 |
| PCSK1 | 3.1585443 |
| BMP6 | 2.5192003 |
| EDN1 | 3.805765 |
| CTGF | 2.8271823 |
| TFPI2 | 1.6620069 |
| NRCAM | 3.4262772 |
| CD274 | 1.5687742 |
| FRMD3 | 3.1186438 |

Similarly, RNAs extracted from PK-ane-iPS1-MC, PK-ane-iPS2-MC, PK-ane-iPS3-MC, PK-ane-iPS4-MC, PK-free-iPS1-MC, PK-free-iPS2-MC, and PK-free-iPS3-MC obtained in Example 3 were subjected to measurement of gene expression intensity using a microarray (Affymetrix). Groups with cerebral aneurysm as a complication and groups without cerebral aneurysm as a complication were compared for all the 12 combinations of clones. Gene groups expressed at high levels in all groups with cerebral aneurysm as a complication are shown in Table 4. Also, the ratio of the expression level in PK-ane-iPS1-MC to that in PK-free-iPS2-MC in one case out of the above 12 combinations is shown in Table 4.

TABLE 4
Gene group expressed at high levels in vascular mural cells
obtained via differentiation induction from iPS cells formed
from fibroblast cells of PKD subjects with cerebral aneurysm

| Gene | Fold Change |
|---|---|
| Name | (PK-ane-iPS1-MC/PK-free-iPS2-MC) |
| MYPN | 1.3425403 |
| SIAE | 2.1190434 |
| MMP1 | 4.1157513 |
| HMGA2 | 1.9827896 |
| RPPH1 | 3.5703707 |
| KRTAP4-7 | 2.1406236 |
| KRTAP4-9 | |
| KRTAP4-8 | |
| TFPI2 | 13.641734 |

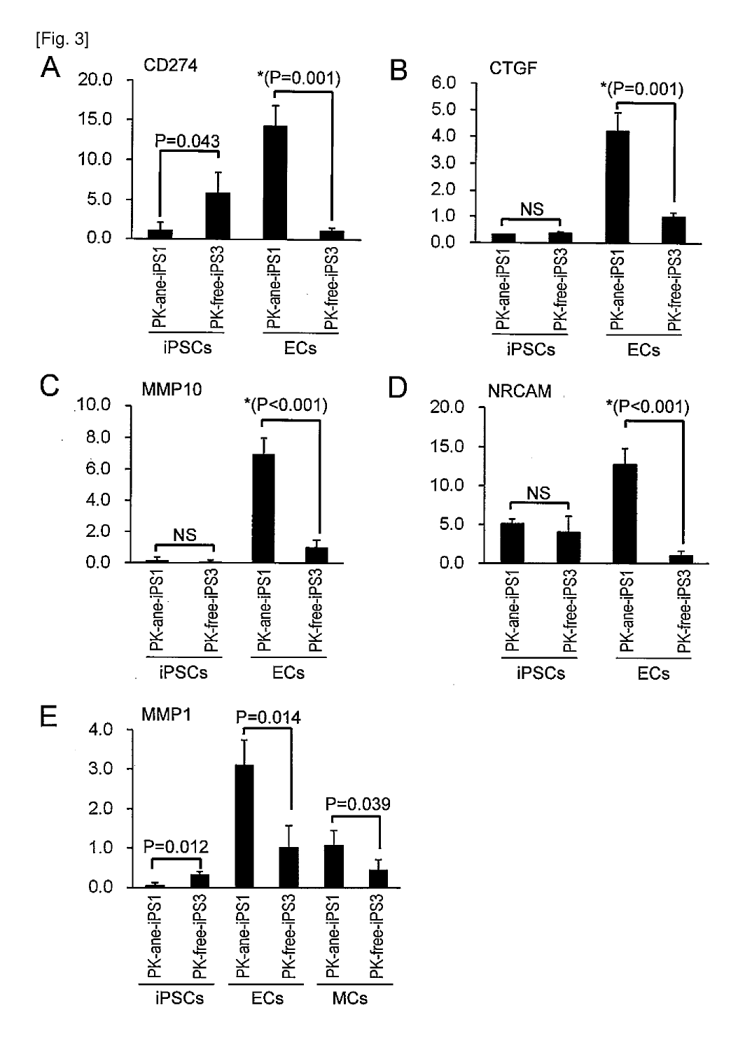

Additionally, the expression levels of CD274, CTGF, MMP10, NRCAM, and MMP1 in endocerial cells (ECs) or mural cells (MCs) derived from PK-ane-iPS 1 and PK-free-iPS3 obtained in Example 2 or 3 were measured by quantitative PCR using primers listed in Table 2. As the result, the expression levels of the indicated genes in the EC or MC induced from iPS cells with cerebral aneurysm were significantly higher than that without cerebral aneurysm, although there was no difference between the expression levels in undifferentiated cells with and without cerebral aneurysm (FIG. 3). These results were corresponding to above.

### INDUSTRIAL APPLICABILITY

The present invention makes it possible to perform a method of examining autosomal dominant polycystic kidney disease or a complication of autosomal dominant polycystic kidney disease, as well as screening for a remedy for the same. Thus, the present invention is medically very useful.

1. A method of examining whether or not a subject has polycystic kidney disease or a risk of developing the disease, comprising the following steps of:
(a) measuring the expression levels of 1 or more genes selected from or all genes of the group consisting of NTNG1, POSTN, TNC, KAL1, BST1, ACAT2, INSIG1, SCD, HSD3B1, KRT7, USP40, and SULT1E1 in a sample from a subject; and
(b) determining that the subject has polycystic kidney disease or a risk of developing the disease when the expression levels of 1 or more genes selected from or all genes of the group consisting of the NTNG1, POSTN, TNC, KAL1, BST1, HSD3B1, KRT7, USP40, and SULT1E1 are lower than those in a control sample, or, determining that the subject has polycystic kidney disease or a risk of developing the disease when the expression levels of ACAT2, INSIG1, or SCD or the three genes are higher than those in the control sample.

2. The method according to claim 1, wherein the sample is at least one type of sample selected from the group consisting of blood, serum, blood plasma, cell extracts, urine, lymph fluids, tissue fluids, ascites, spinal fluids, and other body fluids, or tissues and cells.

3. The method according to claim 1, wherein the sample is a vascular endothelial cell obtained via differentiation induction from an iPS cell formed from an isolated somatic cell of a subject, and the gene in step (a) is selected from the group consisting of NTNG1, POSTN, TNC, KAL1, BST1, ACAT2, INSIG1, and SCD.

4. The method according to claim 1, wherein the sample is a vascular mural cell obtained via differentiation induction from an iPS cell formed from an isolated somatic cell of a subject, and the gene in step (a) is selected from the group consisting of HSD3B1, KRT7, USP40, and SULT1E1.

5. The method according to claim 1, wherein the step of measuring the expression level of the gene is a step of measuring the amount of mRNA, cRNA, or cDNA of the gene or the amount of a protein encoded by the gene in the sample.

6. A method of examining whether or not a subject has a complication of polycystic kidney disease or a risk of developing the complication, comprising the following steps of:
(a) measuring the expression levels of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE in a sample from a subject with polycystic kidney disease; and
(b) determining that the subject has a complication of polycystic kidney disease or a risk of developing the complication when the expression levels of 1 or more genes selected from or all genes of the group consisting of NTNG1, POSTN, TNC, KAL1, BST1, HSD3B1, KRT7, USP40, and SULT1E1 are lower than those in a control sample, or, determining that a subject has a complication of polycystic kidney disease or a risk of developing the complication when the expression levels of 1 or more genes selected from or all genes of the group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE are higher than those in the control sample.

7. The method according to claim 6, wherein the complication is cerebral aneurysm.

8. The method according to claim 6, wherein the sample is at least one type of sample selected from the group consisting of blood, serum, blood plasma, cell extracts, urine, lymph fluids, tissue fluids, ascites, spinal fluids, and other body fluids, or tissues and cells.

9. The method according to claim 6, wherein the sample is a vascular endothelial cell obtained via differentiation induction from an iPS cell formed from an isolated somatic cell of a subject; and wherein the gene in step (a) is 1 or more genes selected from or all genes of the group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2.

10. The method according to claim 6, wherein the sample is a vascular mural cell obtained via differentiation induction from an iPS cell formed from an isolated somatic cell of a subject; and wherein the gene in step (a) is 1 or more genes selected from or all genes of the group consisting of HSD3B1, KRT7, USP40, SULT1E1, MMP1, TFPI2, HMGA2, KRTAP4-7, KRTAP4-8, KRTAP4-9, MYPN, RPPH1, and SIAE.

11. The method according to claim 6, wherein the step of measuring the expression level of the gene is a step of measuring the amount of the mRNA, cRNA, or cDNA of the gene or the amount of the protein encoded by the gene.

12. A disease marker for examining a complication of polycystic kidney disease or polycystic kidney disease, comprising:
a polynucleotide(s) and/or a polynucleotide(s) complementary thereto, which has/have at least 15 continuous nucleotides in the nucleotide sequence(s) of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or an antibody (or antibodies) or a fragment(s) thereof, which recognizes a protein(s) encoded by 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

13. The disease marker according to claim 12, wherein the complication is cerebral aneurysm.

14. The disease marker according to claim 13, comprising:
a polynucleotide(s) and/or polynucleotide(s) complementary thereto, which has/have at least 15 continuous nucleotides in the nucleotide sequence(s) of 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or, an antibody (or antibodies) or a fragment(s) thereof, which recognizes a protein(s) encoded by 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

15. A kit for examining polycystic kidney disease or a complication of polycystic kidney disease, comprising:
a polynucleotide(s) and/or a polynucleotide(s) complementary thereto, which has/have at least 15 continuous nucleotides in the nucleotide sequence(s) of 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or an antibody (or antibodies) or a fragment(s) thereof, which recognizes a protein(s) encoded by 1 or more genes selected from or all genes of the group consisting of NTNG1, POSTN, TNC, KAL1, BST1, ACAT2, INSIG1, SCD, HSD3B1, KRT7, USP40, SULT1E1, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

16. The kit according to claim 15, wherein the complication is cerebral aneurysm.

17. The kit according to claim 16, comprising:
a polynucleotide(s) and/or a polynucleotide(s) complementary thereto, which has/have at least 15 continuous nucleotides in the nucleotide sequence(s) of 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE; and/or
an antibody (or antibodies) or a fragment(s) thereof, which recognizes a protein(s) encoded by 1 or more genes selected from or all genes of the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE.

18. A method of screening for a therapeutic agent or a preventive agent for polycystic kidney disease, comprising the following steps of:
(a) bringing each of candidate substances into contact with a vascular endothelial cell obtained via differentiation induction from an iPS cell formed from an isolated somatic cell of a subject with polycystic kidney disease;
(b) measuring the expression levels or the transcriptional activity of 1 or more genes selected from or all genes of the group consisting of NTNG1, POSTN, TNC, KAL1, BST1, ACAT2, INSIG1, and SCD; and
(c) selecting a candidate substance as a therapeutic agent or a preventive agent for polycystic kidney disease, when the expression levels or the transcriptional activity of 1 or more genes selected from or all genes of the group consisting of NTNG1, POSTN, TNC, KAL1, and BST1 exhibit an increase compared with a case in which the candidate substance is not brought into contact, or,
when the expression levels or the transcriptional activity of 1 or more genes selected from or all genes of the group consisting of ACAT2, INSIG1, and SCD exhibit a decrease compared with a case in which the candidate substance is not brought into contact.

19. A method of screening for a therapeutic agent or a preventive agent for polycystic kidney disease, comprising the following steps of:
(a) bringing each of candidate substances into contact with a vascular mural cell obtained via differentiation induction from an iPS cell formed from an isolated somatic cell of a subject with polycystic kidney disease;
(b) measuring the expression levels or the transcriptional activity of 1 or more genes selected from or all genes of the group consisting of HSD3B1, KRT7, USP40, and SULT1E1; and
(c) selecting a candidate substance as a therapeutic agent or a preventive agent for polycystic kidney disease when the expression levels or the transcriptional activity exhibit an increase compared with a case in which the candidate substance is not brought into contact.

20. The screening method according to claim 18, wherein the step of measuring the expression level of the gene is a step of measuring the amount of mRNA, cRNA, or cDNA of the gene.

21. A method of screening for a therapeutic agent or a preventive agent for a complication of polycystic kidney disease, comprising the following steps of:
(a) bringing each of candidate substances into contact with a vascular endothelial cell obtained via differentiation induction from an iPS cell formed from an isolated somatic cell of a subject with a complication of polycystic kidney disease;
(b) measuring the expression levels or the transcriptional activity of 1 or more genes selected from or all genes of the group consisting of NTNG1, POSTN, TNC, KAL1, BST1, ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2; and
(c) selecting a candidate substance as a therapeutic agent or a preventive agent for a complication of polycystic kidney disease, when the expression levels or the transcriptional activity of 1 or more genes selected from or all genes of the group consisting of NTNG1, POSTN, TNC, KAL1, and BST1 exhibit an increase compared with a case in which the candidate substance is not brought into contact, or
when the expression level or the transcriptional activity of 1 or more genes selected from or all genes of the group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 exhibit a decrease compared with a case in which the candidate substance is not brought into contact.

22. A method of screening for a therapeutic agent or a preventive agent for a complication of polycystic kidney disease, comprising the following steps of:
(a) bringing each of candidate substances into contact with a vascular mural cell obtained via differentiation induction from an iPS cell formed from an isolated somatic cell of a subject with a complication of polycystic kidney disease;
(b) measuring the expression levels or the transcriptional activity of 1 or more genes selected from or all genes of the group consisting of HSD3B1, KRT7, USP40, SULT1E1, MMP1, TFPI2, HMGA2, KRTAP4-7, KRTAP4-8, KRTAP4-9, MYPN, RPPH1, and SIAE; and
(c) selecting a candidate substance as a therapeutic agent or a preventive agent for a complication of polycystic kidney disease, when the expression levels or the transcriptional activity of 1 or more genes selected from or all genes of the group consisting of HSD3B1, KRT7, USP40, and SULT1E1 exhibit an increase compared with a case in which the candidate substance is not brought into contact, or,
when the expression levels or the transcriptional activity of 1 or more genes selected from or all genes of the group consisting of MMP1, TFPI2, HMGA2, KRTAP4-7, KRTAP4-8, KRTAP4-9, MYPN, RPPH1, and SIAE exhibit a decrease compared with a case in which the candidate substance is not brought into contact.

23. The screening method according to claim 21, wherein the complication is cerebral aneurysm.

24. The screening method according to claim 21, wherein the step of measuring the expression level of the gene is a step of measuring the amount of mRNA, cRNA, or cDNA of the gene.

25. A composition for treating or preventing polycystic kidney disease or a complication of polycystic kidney disease, comprising a pharmaceutically effective amount(s) of an antisense oligonucleotide(s), or siRNA(s) or shRNA(s) against at least one nucleic acid selected from the group consisting of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE genes or transcription products thereof.

26. The composition according to claim 25, wherein the complication is cerebral aneurysm.

27. The composition according to claim 26, comprising a pharmaceutically effective amount(s) of an antisense oligonucleotide(s), or siRNA(s) or shRNA(s) against at least one nucleic acid selected from the group consisting of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, TFPI2, HMGA2, KRTAP4-7, KRTAP4-8, KRTAP4-9, MYPN, RPPH1, and SIAE genes or transcription products thereof.

28. A vascular endothelial cell obtained via differentiation induction from an iPS cell formed from an isolated somatic cell of a subject suffered from polycystic kidney disease and having cerebral aneurysm as a complication, wherein the expression levels of NTNG1, POSTN, TNC, KAL1, and BST1 are lower and the expression levels of ACAT2, INSIG1, SCD, BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are higher than those in a vascular endothelial cell obtained via differentiation induction from an iPS cell formed from an isolated somatic cell of a healthy subject.

29. A vascular endothelial cell obtained via differentiation induction from an iPS cell formed from an isolated somatic cell of a subject suffered from polycystic kidney disease and having cerebral aneurysm as a complication, wherein the expression levels of BMP6, CD274, CTGF, E2F7, EDN1, FAM43A, FRMD3, MMP10, MYEOV, NR2F1, NRCAM, PCSK1, PLXNA2, SLC30A3, SNAI1, SPOCD1, MMP1, and TFPI2 are high.

30. A vascular mural cell obtained via differentiation induction from an iPS cell formed from an isolated somatic cell of a subject suffered from polycystic kidney disease and having cerebral aneurysm as a complication, wherein the expression levels of HSD3B1, KRT7, USP40, and SULT1E1 are lower than those in vascular mural cells obtained via differentiation induction from iPS cells formed from a somatic cell of a healthy subject, and the expression levels of MMP1, TFPI2, HMGA2, KRTAP4-7, KRTAP4-8, KRTAP4-9, MYPN, RPPH1, and SIAE are higher than those in a vascular mural cell obtained via differentiation induction from an iPS cell formed from an isolated somatic cell of a healthy subject.

31. A vascular mural cell obtained via differentiation induction from an iPS cell formed from an isolated somatic cell of a subject suffered from polycystic kidney disease and having cerebral aneurysm as a complication, wherein the expression levels of MMP1, TFPI2, HMGA2, KRTAP4-7, KRTAP4-8, KRTAP4-9, MYPN, RPPH1, and SIAE are high.
